(19) United States
(12) Patent Application Publication
(10) Pub. No.: US 2005/0227249 A1
(43) Pub. Date: Oct. 13, 2005

(54) Polymorphisms in the human MDR-1 gene and their use in diagnostic and therapeutic applications

(76) Inventors: Ulrich Brinkmann, Bernried (DE); Sven Hoffmeyer, Eberfing (DE); Michel Eichelbaum, Morr (DE); Ivar Roots, Berlin (DE)

Correspondence Address: FISH & NEAVE IP GROUP;ROPES & GRAY LLP, 1251 AVENUE OF THE AMERICAS FL C3, NEW YORK, NY, 10020-1105, US

(21) Appl. No.: 10/965,348

(22) Filed: Oct. 14, 2004

Related U.S. Application Data
(62) Division of application No. 10/048,427, filed on Jun. 21, 2002, now abandoned.

(30) Foreign Application Priority Data
Feb. 22, 2000 (EP) EP00103361.2
Jul. 30, 1999 (EP) EP99114938.6

(51) Int. Cl. C12Q 1/68; G01N 33/574; C07H 21/04; C12N 15/09; C07K 14/705

(52) U.S. Cl. 435/6; 435/7.23; 435/69.1; 435/320.1; 435/325; 530/350; 536/23.5

(57) ABSTRACT

Described are general means and methods of diagnosing and treating the phenotypic spectrum as well as the overlapping clinical characteristics with several forms of inherited abnormal expression and/or function of the Multi Drug Resistance-1 (MDR-1) gene. In particular, polynucleotides of molecular variant MDR-1 genes which, for example, are associated with unsufficient and/or altered uptake of drugs by a target cell, and vectors comprising such polynucleotides are provided. Furthermore, host cells comprising such polynucleotides or vectors and their use for the production of variant MDR-1 proteins are described. In addition, variant MDR-1 proteins and antibodies specifically recognizing such proteins as well as concerns transgenic non-human animals comprising the above-described polynucleotide or vectors are provided. Described are also methods for identifying and obtaining inhibitors for therapy of disorders related to the malfunction of the MDR-1 gene as well as methods of diagnosing the status of such disorders. Pharmaceutical and diagnostic compositions comprising the above-described polynucleotides, vectors, proteins, antibodies and inhibitors by the above-described method are provided. Said compositions are particularly useful for diagnosing and treating various diseases with drugs that are substrates, inhibitors or modulators of the MDR-1 gene product.

### FIELD OF THE INVENTION

The present invention relates generally to means and methods of diagnosing and treating the phenotypic spectrum as well as the overlapping clinical characteristics with several forms of inherited abnormal expression and/or function of the Multi Drug Resistance-1 (MDR-1) gene. In particular, the present invention relates to polynucleotides of molecular variant MDR-1 genes which, for example, are associated with unsufficient and/or altered uptake of drugs by a target cell, and to vectors comprising such polynucleotides. Furthermore, the present invention relates to host cells comprising such polynucleotides or vectors and their use for the production of variant MDR-1 proteins. In addition, the present invention relates to variant MDR-1 proteins and antibodies specifically recognizing such proteins. The present invention also concerns transgenic non-human animals comprising the above-described polynucleotide or vectors. Moreover, the present invention relates to methods for identifying and obtaining drug candidates and inhibitors for therapy of disorders related to the malfunction of the MDR-1 gene as well as to methods of diagnosing the status of such disorders. The present invention furthermore provides pharmaceutical and diagnostic compositions comprising the above-described polynucleotides, vectors, proteins, antibodies and drugs and inhibitors obtainable by the above-described method. Said compositions are particularly useful for diagnosing and treating various diseases with drugs that are substrates, inhibitors or modulators of the MDR-1 gene or its product.

Several documents are cited throughout the text of this specification. Each of the documents cited herein (including any manufacturer's specifications, instructions, etc.) are hereby incorporated herein by reference; however, there is no admission that any document cited is indeed prior art as to the present invention.

### BACKGROUND OF THE INVENTION

The human MDR-1 gene encodes an integral membrane protein whose function is the energy dependent transport of different substances from the inside of cells and cell membranes to the outside of the cell. While the normal physiological function of MDR-1 is most likely the protection of cells from toxic substances, it is also known that many substrates of the MDR-1 transporter are drugs that have been developed for the treatment of human diseases. Because of that, the degree of expression and the functionality of the MDR-1 gene product can directly affect the effectiveness of any drug that serves as a substrate of MDR-1. For example, it is well known that the expression levels, and hence the degree of the function of MDR-1, directly affects the effectiveness of anti-tumor drugs in cancer therapy. In fact, the gene name “MDR” stands for Multi-Drug-Resistance, reflecting the observance that the protein encoded by this gene causes cancer cells to become refractory to the treatment with many drugs, all of which are substrates of the MDR-1 transporter.

The MDR-1 gene is expressed not only on certain cancer cells where it may directly affect the therapeutic effectiveness of drugs by providing a protective barrier against drug entry, but also on different non-malignant cells in various organs, e.g. in the colon and at the blood brain barrier. Also in these cells MDR-1 can affect the activity and availability of drugs. For example, MDR-1 in colon can control or modulate the degree of drug uptake from the colon following oral drug intake. MDR-1 at the blood-brain barrier may also influence or control the degree to which MDR-1 substrates can be taken up into the brain. Here, elevated MDR-1 activity may prevent the uptake of sufficient amounts of desired brain-drugs into the brain, or vice versa, MDR-1 variants with reduced activity towards certain drugs might lead to abnormally increased accumulation in the brain, leading to undesired or even dangerous drug side effects.

The common factor that controls MDR-1 dependent transport in malignant as well as normal cells and tissues is the activity of MDR-1. The MDR-1 activity in turn is dependent (i) on the levels of expression of the MDR-1 gene which determines the amount of MDR-1 protein that is synthesized in the cells, and (ii) on the functionality of the synthesized MDR-1 protein, i.e. which substrates are recognized and transported out of the cell with which effectiveness.

The first of these parameter, the level of expression of MDR-1, has been intensively analyzed, particularly because the sensitivity of tumor cells towards cancer chemotherapy often correlates inversely with upregulation of MDR-expression: high MDR-1 expression correlates often with unsufficient effectiveness of cancer chemotherapy. Although the observed MDR-1 overexpression can partially be attributed to MDR-1 gene amplifications, it is known that other so far undetermined reasons must also exist, among them possibly allelic differences. Small differences in the MDR-1 gene sequences in individuals may be causative for different levels of MDR-1 gene expression. Target regions in the human genome where sequence differences might exist that directly influence MDR-1 gene expression would be the control regions of gene expression: the promoter and enhancer regions of MDR-1 and regions that influence the mode or efficacy of splicing of MDR-1 pre-mRNAs. In addition, expression levels may be influenced by structural changes in the genome, such as methylation, general chromatin alterations and other factors that are linked to MDR-1, in the region directly at or surrounding the MDR-1 gene. It is very difficult to directly find such linked factors or sequences and prove their mechanism of gene activation or repression. However, the linear structure of the human genome on defined chromosomes opens the possibility to utilize identified polymorphisms, which by themselves are not directly influencing expression levels of genes, as marker for other so far unidentified changes in and around the MDR-1 gene that affect the expression levels. This effect is known as linkage: defined alleles and base variations can serve as a marker for an important phenotype even if these changes by themselves are not causative for that phenotype.

The second parameter, the functionality of the synthesized MDR-1 protein, i.e. which substrates are recognized and transported out of the cell with which effectiveness, is predominantly determined by the amino acid sequence of the protein that is encoded by the MDR-1 allele. It is well known that amino acid changes may alter the functionality of proteins. Examples for naturally occurring variations, i.e. different alleles that have a direct impact on the actions of various drugs are, e.g., cytochrome P450 polymorphisms, or polymorphisms in TPMT, APOE, and a variety of other genes. Also, tumor related variations, e.g., in the p53 gene are known to mediate such phenotypes. So far only some polymorphism in the MDR-1 gene have been described, and been correlated with clinical effects (Mickley, Blood 91 (1998), 1749-1756). A major question remains in this field whether more of such polymorphisms exist and, if so, whether these can be correlated with drug activity and/or drug side effects. Experiments with artificially introduced mutations in the MDR-1 gene show unambiguously that MDR-1 reacts quite sensitive to amino acid exchanges. It has been shown that artificial mutations in the MDR-1 gene that translate into protein changes can alter the substrate spectrum, effectiveness of substrate transport, control of transport, and also the sensitivity of MDR-1 towards inhibition with specific inhibitory substances. It is clear that naturally occurring mutations, if they exist can have similar effects. It is unknown, however, how many of such variations exist, and with what frequency and at what positions in the human MDR-1 gene.

Accordingly, means and methods for diagnosing and treating a variety of forms of multidrug resistance which result from MDR-1 gene polymorphisms, and sensitivity interfering, e.g., with chemotherapeutic treatment of diseases, in particular cancer, was hitherto not available but are nevertheless highly desirable.

Thus, the technical problem of the present invention is to comply with the needs described above.

The solution to this technical problem is achieved by providing the embodiments characterized in the claims.

### SUMMARY OF THE INVENTION

The present invention is based on the finding of novel, so far unknown variations in the nucleotide sequences of the human MDR-1 (Multi Drug Resistance) gene and the population distribution of these alleles. Based upon the knowledge of these novel sequences and MDR-1 gene base deviations, diagnostic tests and reagents for such tests were designed for the specific detection and genotyping of MDR-1 alleles in humans, including homozygous as well as heterozygous, frequent as well as rare alleles of the MDR-1 gene. The determination of the MDR-1 gene allele status of humans with such tests is useful for the therapy of various diseases with drugs that are substrates, inhibitors or modulators of the MDR-1 gene product.

In a first embodiment, the invention provides polynucleotides of molecular variant MDR-1 genes and embodiments related thereto such as vectors, host cells, variant MDR-1 proteins and methods for producing the same.

In yet another embodiment, the invention provides methods for identifying and obtaining drug candidates and inhibitors of MDR-1 for therapy of disorders related to acquired multidrug resistance or sensitivity as well as methods of diagnosing the status of such disorders.

In a further embodiment, the invention provides pharmaceutical and diagnostic compositions comprising the above-described polynucleotides, vectors containing the same, proteins, antibodies thereto and drugs and inhibitors obtainable by the above-described method.

The pharmaceutical and diagnostic compositions, methods and uses of the invention are useful for the diagnosis and treatment of inherited drug resistance in tumors and other diseases the therapy of which is dependent on drug treatment. The novel variant forms of MDR-1 genes according to the invention provide the potential for the development of a pharmacodynamic profile of drugs and prodrugs for a given patient.

### DESCRIPTION OF THE INVENTION

The finding and characterization of variations in the MDR-1 gene, and diagnostic tests for the discrimination of different MDR-1 alleles in human individuals provide a very potent tool for improving the therapy of diseases with drugs that are targets of the MDR-1 gene product, and whose cellular uptake is therefore dependent on MDR-1. The diagnosis of the individual allelic MDR-1 status permits a more focused therapy, e.g., by opening the possibility to apply individual dose regimens of drugs. It may also be useful as prognostic tool for therapy outcome, certainly an improved approach over the use of general MDR-expression as prognostic maker. Furthermore, diagnostic tests to genotype MDR-1, and novel MDR-1 variants, will not only improve therapy established drugs and help to correlate genotypes with drug activity or side effects. These tests and sequences also provide reagents for the development of novel inhibitors that specifically modulate the activity of the individual types of MDR-1. The feasibility to use specific inhibitors of individual (artificially created) MDR-variants, and their potential therapeutic application, has, for example, recently been demonstrated in a model system (Moscow J. A. et al., Blood 94 (1999), 52-61; Dey S. et al., Biochemistry 38 (1999), 6630-6639).

Thus, the present invention provides a novel way to exploit molecular biology and pharmalogical research for drug therapy while bypassing their potential detrimental effects which are due to expression of variant MDR-1 genes.

Accordingly, the invention relates to a polynucleotide selected from the group consisting of:
(a) a polynucleotide having the nucleic acid sequence of any one of SEQ ID NOs: 73, 74, 79, 80, 85, 86, 91, 92, 97, 98, 101, 106, 107, 112, 113, 116, 119, 122, 154, 155, 160, 161, 166, 167, 172, 173, 178, 179, 184, 185, 190, 191, 196, 197, 202, 203, 208, 209, 214, 215, 220, 221, 226, 227, 232, 233, 238, 239, 244, 245, 250, 251, 256, 257, 262, 263, 268, 269, 274, 275, 280, 281, 286, 287, 292, 293, 298, 299, 304, 305, 310, 311, 316, 317, 322, 323, 328, 329, 334, 335, 340, 341, 346, 347, 352, 353, 358, 359, 364, 365, 370 or 371;
(b) a polynucleotide encoding a polypeptide having the amino acid sequence of any one of SEQ ID NOs: 372, 373 374 or 375;
(c) a polynucleotide encoding a molecular variant Multi Drug Resistance (MDR)-1 polypeptide, wherein said polynucleotide is having at a position corresponding to position 140837, 141530, 141590, 171466, 171512 or 175068 of the MDR-1 gene (Accession No: AC002457), at a position corresponding to position 101 or 308 of the MDR-1 gene (Accession No: M29432 or J05168), at a position corresponding to position 83946 of the MDR-1 gene (Accession No: AC005068), at a position corresponding to position 78170 of the MDR-1 gene (Accession No: AC005068), at a position corresponding to position 176 of the MDR-1 gene (Accession No: M29445 or J05168), at a position corresponding to position 171456, 171404 or 175074 of the MDR-1 gene (Accession No: AC002457), at a position corresponding to position 77811 of the MDR-1 gene (Accession No: AC005068) or at a position corresponding to position 137 of the MDR-1 gene (Accession No: M29445 or J05168) a nucleotide exchange, a nucleotide deletion, an additional nucleotide or an additional nucleotide and a nucleotide exchange;
(d) a polynucleotide encoding a molecular variant MDR-1 polypeptide, wherein said polynucleotide is having at a position corresponding to position 140837, 171512, 171456, 171404, 139119, 139619, 140490 or 171511 of the MDR-1 gene (Accession No: AC002457) a C, at a position corresponding to position 141530, 139177, 139479, 140118, 140568, 140727 or 174901 of the MDR-1 gene (Accession No: AC002457) a A, at a position corresponding to position 141590, 139015, 140216, 140595, 175142 or 175180 of the MDR-1 gene (Accession No: AC002457) a G, at a position corresponding to position 171466, 175068, 175074, 139064, 139276, 140576 or 145984 of the MDR-1 gene (Accession No: AC002457) a T, at a position corresponding to position 101 of the MDR-1 gene (Accession No: M29432 or J05168) a A, at a position corresponding to position 308 of the MDR-1 gene (Accession No: M29432 or J05168) a T, at a position corresponding to position 83946, 78170, 70237 or 70200 of the MDR-1 gene (Accession No: AC005068) a T, at a position corresponding to position 77811, 84032 or 73252 of the MDR-1 gene (Accession No: AC005068) a G, at a position corresponding to position 84701, 84074, 84119, 83973, 70371, 70253, 70204 or 43162 of the MDR-1 gene (Accession No: AC005068) a A, at a position corresponding to position 43263 of the MDR-1 gene (Accession No: AC005068) a C or at a position corresponding to position 176 or 137 of the MDR-1 gene (Accession No: M29445 or J05168) a T;
(e) a polynucleotide encoding a molecular variant MDR-1 peptide, wherein said polypeptide comprises an amino acid substitution at position 21, 103 or 400 of the MDR-1 polypeptide (Accession No: P08183); and
(f) a polynucleotide encoding a molecular variant MDR-1 polypeptide, wherein said polypeptide comprises an amino acid substitution of N to D at position 21, F to S at position 103, F to L at position 103 or S to N at position 400 of the MDR-1 polypeptide (Accession No: P08183).

In the context of the present invention the term “molecular variant” MDR-1 gene or protein as used herein means that said MDR-1 gene or protein differs from the wild type MDR-1 gene or protein (Genomic sequences of the MDR-1 gene are described, for examples, for exons 1-7: Accession number AC002457; for exon 8: Accession number M29429, J05168, AC005068; for exon 9: Accession number M29430, J05168, AC005068; for exon 10: Accession number M29431, J05168, AC005068; for exon 11 to 13: Accession number M29432, J05168 and AC005068; for exon 14: Accession number M29433, J05168, AC005068; for exon 15: Accession number M29434, J05168, AC005068; for exon 16: Accession number M29435, J05168, AC005068; for exon 17: Accession number M29436, J05168, AC005068; for exon 18: Accession number M29437, J05168, AC005068; for exon 19: Accession number M29438, J05168, AC005068; for exon 20: Accession number M29439, J05168, AC005068; for exon 21: Accession number M29440, J05168, AC005068; for exon 22: Accession number M29441, J05168, AC005068; for exon 23: Accession number M29442, J05168, AC005068; for exon 24: Accession number M29443, J05168, AC005068; for exon 25: Accession number M29444, J05168, AC005068; for exon 26: Accession number M29445, J05168, AF016535, AC005068; for exon 27: Accession number M29446, J05168, AC005068; for exon 28: Accession number M29447, J05168, AC005068) by way of nucleotide substitution(s), addition(s) and/or deletion(s). Preferably, said nucleotide substitution(s) result(s) in a corresponding change in the amino acid sequence of the MDR-1 protein.

The term “corresponding” as used herein means that a position is not only determined by the number of the preceding nucleotides and amino acids, respectively. The position of a given nucleotide or amino acid in accordance with the present invention which may be deleted, substituted or comprise one or more additional nucleotide(s) may vary due to deletions or additional nucleotides or amino acids elsewhere in the gene or the polypeptide. Thus, under a “corresponding position” in accordance with the present invention it is to be understood that nucleotides or amino acids may differ in the indicated number but may still have similar neighboring nucleotides or amino acids. Said nucleotides or amino acids which may be exchanged, deleted or comprise additional nucleotides or amino acids are also comprised by the term “corresponding position”. Said nucleotides or amino acids may for instance together with their neighbors form sequences which may be involved in the regulation of gene expression, stability of the corresponding RNA or RNA editing, as well as encode functional domains or motifs of the protein of the invention.

In accordance with the present invention, the mode and population distribution of novel so far unidentified genetic variations in the MDR-1 gene have been analyzed by sequence analysis of relevant regions of the human MDR-1 gene from many different individuals. It is a well known fact that genomic DNA of individuals, which harbor the individual genetic makeup of all genes, including MDR-1 can easily be purified from individual blood samples. These individual DNA samples are then used for the analysis of the sequence composition of the MDR-1 gene alleles that are present in the individual which provided the blood sample. The sequence analysis was carried out by PCR amplification of relevant regions of the MDR-1 gene, subsequent purification of the PCR products, followed by automated DNA sequencing with established methods (ABI dyeterminator cycle sequencing). One important parameter that had to be considered in the attempt to determine the individual MDR-1 genotype and identify novel MDR-1 variants by direct DNA-sequencing of PCR-products from human blood genomic DNA is the fact that each human harbors (usually, with very few abnormal exceptions) two gene copies of each autosomal gene (diploidy). Because of that, great care had to be taken in the evaluation of the sequences to be able to identify unambiguously not only homozygous sequence variations but also heterozygous variations. The details of the different steps in the identification and characterization of novel MDR-1 gene polymorphisms (homozygous and heterozygous) are described in the examples 1 and 2 below.

The mutations in the MDR-1 gene detected in accordance with the present invention are illustrated in FIG. 2 (indicated by an arrow). The methods of the mutation analysis followed standard protocols and are described in detail in the examples. In general such methods to be used in accordance with the present invention for evaluating the phenotypic spectrum as well as the overlapping clinical characteristics with other forms of multidrug resistance and altered tolerance to drugs in patients with mutations in the MDR-1 gene encompass for example haplotype analysis, single-strand conformation polymorphism analysis (SSCA), PCR and direct sequencing; see also Mickley (1998), and references cited therein. On the basis of thorough clinical characterization of many patients the phenotypes can then be correlated to these mutations as well as to mutations that had been described earlier.

As is evident to the person skilled in the art this new molecular genetic knowledge can now be used to exactly characterize the genotype of the index patient where a given drug takes an unusual effect and of his family.

Over the past 20 years, genetic heterogeneity has been increasingly recognized as a significant source of variation in drug response. Many scientific communications (Meyer, Ann. Rev. Pharmacol. Toxicol. 37 (1997), 269-296 and West, J. Clin. Pharmacol. 37 (1997), 635-648) have clearly shown that some drugs work better or may even be highly toxic in some patients than in others and that these variations in patient's responses to drugs can be related to molecular basis. This “pharmacogenomic” concept spots correlations between responses to drugs and genetic profiles of patient's (Marshall, Nature Biotechnology, 15 (1997), 954-957; Marshall, Nature Biotechnology, 15 (1997), 1249-1252).

In this context of population variability with regard to drug therapy, pharmacogenomics has been proposed as a tool useful in the identification and selection of patients which can respond to a particular drug without side effects. This identification/selection can be based upon molecular diagnosis of genetic polymorphisms by genotyping DNA from leukocytes in the blood of patient, for example, and characterization of disease (Bertz, Clin. Pharmacokinet. 32 (1997), 210-256; Engel, J. Chromatogra. B. Biomed. Appl. 678 (1996), 93-103). For the founders of health care, such as health maintenance organizations in the US and government public health services in many European countries, this pharmacogenomics approach can represent a way of both improving health care and reducing overheads because there is a large cost to unnecessary drugs, ineffective drugs and drugs with side effects.

The mutations in the variant MDR-1 genes sometime result in amino acid deletion(s), insertion(s) and in particular in substitution(s) either alone or in combination. It is of course also possible to genetically engineer such mutations in wild type genes or other mutant forms. Methods for introducing such modifications in the DNA sequence of MDR-1 gene are well known to the person skilled in the art; see, e.g., Sambrook, Molecular Cloning A Laboratory Manual, Cold Spring Harbor Laboratory (1989) N.Y.

In a preferred embodiment of the invention, the above described polynucleotide encodes a variant MDR-1 protein or fragment thereof, e.g., comprising one or more epitopes of the amino acid sequence encoded by SEQ ID NOs: 85, 97, 106 or 274.

For the investigation of the nature of the alterations in the amino acid sequence of the MDR-1 protein computer programs may be used such as BRASMOL that are obtainable from the Internet. Furthermore, folding simulations and computer redesign of structural motifs can be performed using other appropriate computer programs (Olszewski, Proteins 25 (1996), 286-299; Hoffman, Comput. Appl. Biosci. 11 (1995), 675-679). Computers can be used for the conformational and energetic analysis of detailed protein models (Monge, J. Mol. Biol. 247 (1995), 995-1012; Renouf, Adv. Exp. Med. Biol. 376 (1995), 37-45). These analysis can be used for the identification of the influence of a particular mutation on binding and/or transport of drugs.

Usually, said amino acid deletion, addition or substitution in the amino acid sequence of the protein encoded by the polynucleotide of the invention is due to one or more nucleotide substitution, insertion or deletion, or any combinations thereof. Preferably said nucleotide substitution, insertion or deletion results in an amino acid substitution of Asn21 to Asp in exon 2, Phe103 to Ser or Leu in exon 5 and/or Ser400 to Asn in exon 11 of the MDR-1 gene.

The polynucleotide of the invention may further comprise at least one nucleotide and optionally amino acid deletion, addition and/or substitution other than those specified hereinabove, for example those described in the prior art; e.g., Mickley (1998). This embodiment of the present invention allows the study of synergistic effects of the mutations in the MDR-1 gene on the pharmalogical profile of drugs in patients who bear such mutant forms of the gene or similar mutant forms that can be mimicked by the above described proteins. It is expected that the analysis of said synergistic effects provides deeper insights into the onset of multidrug resistant phenotypes of certain forms of cancer. From said deeper insight the development of diagnostic and pharmaceutical compositions related to cancer will greatly benefit.

Thus, in a preferred embodiment, the present invention relates to polynucleotides of molecular variant MDR-1 genes, wherein the nucleotide deletion, addition and/or substitution result in altered expression of the variant MDR-1 gene compared to the corresponding wild type gene.

The polynucleotide of the invention may be, e.g., DNA, cDNA, genomic DNA, RNA or synthetically produced DNA or RNA or a recombinantly produced chimeric nucleic acid molecule comprising any of those polynucleotides either alone or in combination. Preferably said polynucleotide is part of a vector, particularly plasmids, cosmids, viruses and bacteriophages used conventionally in genetic engineering that comprise a polynucleotide of the invention. Such vectors may comprise further genes such as marker genes which allow for the selection of said vector in a suitable host cell and under suitable conditions.

In a further preferred embodiment of the vector of the invention, the polynucleotide of the invention is operatively linked to expression control sequences allowing expression in prokaryotic or eukaryotic cells. Expression of said polynucleotide comprises transcription of the polynucleotide, preferably into a translatable mRNA. Regulatory elements ensuring expression in eukaryotic cells, preferably mammalian cells, are well known to those skilled in the art. They usually comprise regulatory sequences ensuring initiation of transcription and optionally poly-A signals ensuring termination of transcription and stabilization of the transcript. Additional regulatory elements may include transcriptional as well as translational enhancers. Possible regulatory elements permitting expression in prokaryotic host cells comprise, e.g., the lac, trp or tac promoter in E. coli, and examples for regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells. Beside elements which are responsible for the initiation of transcription such regulatory elements may also comprise transcription termination signals, such as the SV40-poly-A site or the tk-poly-A site, downstream of the polynucleotide. In this context, suitable expression vectors are known in the art such as Okayama-Berg cDNA expression vector pcDV1 (Pharmacia), pCDM8, pRc/CMV, pcDNA1, pcDNA3 (In-vitrogene), pSPORT1 (GIBCO BRL). Preferably, said vector is an expression vector and/or a gene transfer or targeting vector. Expression vectors derived from viruses such as retroviruses, vaccinia virus, adeno-associated virus, herpes viruses, or bovine papilloma virus, may be used for delivery of the polynucleotides or vector of the invention into targeted cell population. Methods which are well known to those skilled in the art can be used to construct recombinant viral vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994). Alternatively, the polynucleotides and vectors of the invention can be reconstituted into liposomes for delivery to target cells.

The present invention furthermore relates to host cells transformed with a polynucleotide or vector of the invention. Said host cell may be a prokaryotic or eukaryotic cell; see supra. The polynucleotide or vector of the invention which is present in the host cell may either be integrated into the genome of the host cell or it may be maintained extrachromosomally. In this respect, it is also to be understood that the recombinant DNA molecule of the invention can be used for “gene targeting” and/or “gene replacement”, for restoring a mutant gene or for creating a mutant gene via homologous recombination; see for example Mouellic, Proc. Natl. Acad. Sci. USA, 87 (1990), 4712-4716; Joyner, Gene Targeting, A Practical Approach, Oxford University Press.

The host cell can be any prokaryotic or eukaryotic cell, such as a bacterial, insect, fungal, plant, animal or human cell. Preferred fungal cells are, for example, those of the genus Saccharomyces, in particular those of the species S. cerevisiae. The term “prokaryotic” is meant to include all bacteria which can be transformed or transfected with a polynucleotide for the expression of a variant MDR-1 protein or fragment thereof. Prokaryotic hosts may include gram negative as well as gram positive bacteria such as, for example, E. coli, S. typhimurium, Serratia marcescens and Bacillus subtilis. A polynucleotide coding for a mutant form of MDR-1 variant proteins can be used to transform or transfect the host using any of the techniques commonly known to those of ordinary skill in the art. Methods for preparing fused, operably linked genes and expressing them in bacteria or animal cells are well-known in the art (Sambrook, supra). The genetic constructs and methods described therein can be utilized for expression of variant MDR-1 proteins in, e.g., prokaryotic hosts. In general, expression vectors containing promoter sequences which facilitate the efficient transcription of the inserted polynucleotide are used in connection with the host. The expression vector typically contains an origin of replication, a promoter, and a terminator, as well as specific genes which are capable of providing phenotypic selection of the transformed cells. The transformed prokaryotic hosts can be grown in fermentors and cultured according to techniques known in the art to achieve optimal cell growth. The proteins of the invention can then be isolated from the grown medium, cellular lysates, or cellular membrane fractions. The isolation and purification of the microbially or otherwise expressed polypeptides of the invention may be by any conventional means such as, for example, preparative chromatographic separations and immunological separations such as those involving the use of monoclonal or polyclonal antibodies.

Thus, in a further embodiment the invention relates to a method for the production of variant MDR-1 proteins and fragments thereof comprising culturing a host cell as defined above under conditions allowing the expression of the protein and recovering the produced protein or fragment from the culture.

In another embodiment the present invention relates to a method for producing cells capable of expressing a variant MDR-1 gene comprising genetically engineering cells with the polynucleotide or with the vector of the invention. The cells obtainable by the method of the invention can be used, for example, to test drugs according to the methods described in D. L. Spector, R. D. Goldman, L. A. Leinwand, Cells, a Lab manual, CSH Press 1998. Furthermore, the cells can be used to study known drugs and unknown derivatives thereof for their ability to complement the drug transport deficiency caused by mutations in the MDR-1 gene. For these embodiments the host cells preferably lack a wild type allele, preferably both alleles of the MDR-1 gene and/or have at least one mutated from thereof. Alternatively, strong overexpression of a mutated allele over the normal allele and comparison with a recombinant cell line overexpressing the normal allele at a similar level may be used as a screening and analysis system. The cells obtainable by the above-described method may also be used for the screening methods referred to herein below.

Furthermore, the invention relates to a variant MDR-1 protein or fragments thereof encoded by a polynucleotide according to the invention or obtainable by the above-described methods or from cells produced by the method described above. In this context it is also understood that the variant MDR-1 proteins according to the invention may be further modified by conventional methods known in the art. By providing the variant MDR-1 proteins according to the present invention it is also possible to determine the portions relevant for their biological activity or inhibition of the same, namely their drug transport activity.

The present invention furthermore relates to antibodies specifically recognizing a variant MDR-1 protein according to the invention. Advantageously, the antibody specifically recognizes an epitope containing one or more amino acid substitution(s) as defined above

Antibodies against the variant MDR-1 protein of the invention can be prepared by well known methods using a purified protein according to the invention or a (synthetic) fragment derived therefrom as an antigen. Monoclonal antibodies can be prepared, for example, by the techniques as originally described in Köhler and Milstein, Nature 256 (1975), 495, and Galfré, Meth. Enzymol. 73 (1981), 3, which comprise the fusion of mouse myeloma cells to spleen cells derived from immunized mammals. The antibodies can be monoclonal antibodies, polyclonal antibodies or synthetic antibodies as well as fragments of antibodies, such as Fab, Fv or scFv fragments etc. Furthermore, antibodies or fragments thereof to the aforementioned polypeptides can be obtained by using methods which are described, e.g., in Harlow and Lane “Antibodies, A Laboratory Manual”, CSH Press, Cold Spring Harbor, 1988. These antibodies can be used, for example, for the immunoprecipitation and immunolocalization of the variant MDR-1 proteins of the invention as well as for the monitoring of the presence of such variant MDR-1 proteins, for example, in recombinant organisms, and for the identification of compounds interacting with the proteins according to the invention. For example, surface plasmon resonance as employed in the BIAcore system can be used to increase the efficiency of phage antibodies which bind to an epitope of the protein of the invention (Schier, Human Antibodies. Hybridomas 7 (1996), 97-105; Malmborg, J. Immunol. Methods 183 (1995), 7-13).

Furthermore, the present invention relates to nucleic acid molecules which represent or comprise the complementary strand of any of the above described polynucleotides or a part thereof, thus comprising at least one nucleotide difference compared to the corresponding wild type MDR-1 gene nucleotide sequences specified by the above described nucleotide substitutions, deletions and additions. Such a molecule may either be a deoxyribonucleic acid or a ribonucleic acid. Such molecules comprise, for example, antisense RNA. These molecules may furthermore be linked to sequences which when transcribed code for a ribozyme thereby producing a ribozyme which specifically cleaves transcripts of polynucleotides according to the invention.

Furthermore, the present invention relates to a vector comprising a nucleic acid molecule according to the invention. Examples for such vectors are described above. Preferably, the nucleic acid molecule present in the vector is operatively linked to regulatory elements permitting expression in prokaryotic or eukaryotic host cells; see supra.

The present invention also relates to a method for the production of a transgenic non-human animal, preferably transgenic mouse, comprising introduction of a polynucleotide or vector of the invention into a germ cell, an embryonic cell, stem cell or an egg or a cell derived therefrom. The non-human animal can be used in accordance with the method of the invention described below and may be a non-transgenic healthy animal, or may have a disorder, preferably a disorder caused by at least one mutation in the MDR-1 gene. Such transgenic animals are well suited for, e.g., pharmacological studies of drugs in connection with variant forms of the above described variant MDR-1 proteins since these proteins or at least their functional domains are conserved between species in higher eukaryotes, particularly in mammals. Production of transgenic embryos and screening of those can be performed, e.g., as described by A. L. Joyner Ed., Gene Targeting, A Practical Approach (1993), Oxford University Press. The DNA of the embryos can be analyzed using, e.g., Southern blots with an appropriate probe.

The invention also relates to transgenic non-human animals such as transgenic mouse, rats, hamsters, dogs, monkeys, rabbits, pigs, C. elegans and fish such as torpedo fish comprising a polynucleotide or vector of the invention or obtained by the method described above, preferably wherein said polynucleotide or vector is stably integrated into the genome of said non-human animal, preferably such that the presence of said polynucleotide or vector leads to the expression of the variant MDR-1 protein of the invention. It may have one or several copies of the same or different polynucleotides of the variant MDR-1 gene. This animal has numerous utilities, including as a research model for multidrug resistance and therefore, presents a novel and valuable animal in the development of therapies, treatment, etc. for diseases caused by deficiency or failure of drug retention in the cell. Accordingly, in this instance, the mammal is preferably a laboratory animal such as a mouse or rat.

Preferably, the transgenic non-human animal of the invention further comprises at least one inactivated wild type allele of the MDR-1 gene. This embodiment allows for example the study of the interaction of various variant forms of MDR-1 proteins. It might be also desirable to inactivate MDR-1 gene expression or function at a certain stage of development and/or life of the transgenic animal. This can be achieved by using, for example, tissue specific, developmental and/or cell regulated and/or inducible promoters which drive the expression of, e.g., an antisense or ribozyme directed against the RNA transcript of the MDR-1 gene; see also supra. A suitable inducible system is for example tetracycline-regulated gene expression as described, e.g., by Gossen and Bujard (Proc. Natl. Acad. Sci. 89 USA (1992), 5547-5551) and Gossen et al. (Trends Biotech. 12 (1994), 58-62). Similar, the expression of the variant MDR-1 gene may be controlled by such regulatory elements.

With the variant MDR-1 polynucleotides and proteins and vectors of the invention, it is now possible to study in vivo and in vitro the efficiency of drugs in relation to particular mutations in the MDR-1 gene of a patient and the affected phenotype. Furthermore, the variant MDR-1 proteins of the invention can be used to determine the pharmacological profile of drugs and for the identification and preparation of further drugs which may be more effective for the treatment of, e.g., cancer, in particular for the amelioration of certain phenotypes caused by the respective mutations such as those described above.

Thus, a particular object of the present invention concerns drug/pro-drug selection and formulation of pharmaceutical compositions for the treatment of diseases which are amenable to chemotherapy taking into account the polymorphism of the variant form of the MDR-1 gene that cosegregates with the affected phenotype of the patient to be treated. This allows the safe and economic application of drugs which for example were hitherto considered not appropriate for therapy of, e.g., cancer due to either their side effects in some patients and/or their unreliable pharmalogical profile with respect to the same or different phenotype(s) of the disease. The means and methods described herein can be used, for example, to improve dosing recommendations and allows the prescriber to anticipate necessary dose adjustments depending on the considered patient group.

In a further embodiment the present invention relates to a method of identifying and obtaining an MDR-1 inhibitor capable of modulating the activity of a molecular variant of the MDR-1 gene or its gene product comprising the steps of
(a) contacting the variant MDR-1 protein or a cell expressing a molecular variant MDR-1 gene comprising a polynucleotide of the invention in the presence of components capable of providing a detectable signal in response to drug transport, with a compound to be screened under conditions to permit MDR-1 mediated drug transport, and
(b) detecting the presence or absence of a signal or increase of a signal generated from the drug transport, wherein the presence or increase of the signal is indicative for a putative inhibitor.

The term “compound” in a method of the invention includes a single substance or a plurality of substances which may or may not be identical.

Said compound(s) may be chemically synthesized or produced via microbial fermentation but can also be comprised in, for example, samples, e.g., cell extracts from, e.g., plants, animals or microorganisms. Furthermore, said compounds may be known in the art but hitherto not known to be useful as an inhibitor, respectively. The plurality of compounds may be, e.g., added to the culture medium or injected into a cell or non-human animal of the invention.

If a sample containing (a) compound(s) is identified in the method of the invention, then it is either possible to isolate the compound from the original sample identified as containing the compound, in question or one can further subdivide the original sample, for example, if it consists of a plurality of different compounds, so as to reduce the number of different substances per sample and repeat the method with the subdivisions of the original sample. It can then be determined whether said sample or compound displays the desired properties, for example, by the methods described herein or in the literature (Spector et al., Cells manual; see supra). Depending on the complexity of the samples, the steps described above can be performed several times, preferably until the sample identified according to the method of the invention only comprises a limited number of or only one substance(s). Preferably said sample comprises substances of similar chemical and/or physical properties, and most preferably said substances are identical. The methods of the present invention can be easily performed and designed by the person skilled in the art, for example in accordance with other cell based assays described in the prior art or by using and modifying the methods as described herein. Furthermore, the person skilled in the art will readily recognize which further compounds and/or enzymes may be used in order to perform the methods of the invention, for example, enzymes, if necessary, that convert a certain compound into the precursor which in turn represents a substrate for the MDR-1 protein. Such adaptation of the method of the invention is well within the skill of the person skilled in the art and can be performed without undue experimentation.

Compounds which can be used in accordance with the present invention include peptides, proteins, nucleic acids, antibodies, small organic compounds, ligands, peptidomimetics, PNAs and the like. Said compounds can also be functional derivatives or analogues of known drugs such as verapamil or cyclosporin. Methods for the preparation of chemical derivatives and analogues are well known to those skilled in the art and are described in, for example, Beilstein, Handbook of Organic Chemistry, Springer edition New York Inc., 175 Fifth Avenue, New York, N.Y. 10010 U.S.A. and Organic Synthesis, Wiley, New York, USA. Furthermore, said derivatives and analogues can be tested for their effects according to methods known in the art or as described. Furthermore, peptide mimetics and/or computer aided design of appropriate drug derivatives and analogues can be used, for example, according to the methods described below. Such analogs comprise molecules having as the basis structure of known MDR-substrates and/or inhibitors and/or modulators; see infra. Appropriate computer programs can be used for the identification of interactive sites of a putative inhibitor and the MDR-1 protein of the invention by computer assistant searches for complementary structural motifs (Fassina, Immunomethods 5 (1994), 114-120). Further appropriate computer systems for the computer aided design of protein and peptides are described in the prior art, for example, in Berry, Biochem. Soc. Trans. 22 (1994), 1033-1036; Wodak, Ann. N.Y. Acad. Sci. 501 (1987), 1-13; Pabo, Biochemistry 25 (1986), 5987-5991. The results obtained from the above-described computer analysis can be used in combination with the method of the invention for, e.g., optimizing known inhibitors. Appropriate peptidomimetics and other inhibitors can also be identified by the synthesis of peptidomimetic combinatorial libraries through successive chemical modification and testing the resulting compounds, e.g., according to the methods described herein. Methods for the generation and use of peptidomimetic combinatorial libraries are described in the prior art, for example in Ostresh, Methods in Enzymology 267 (1996), 220-234 and Dorner, Bioorg. Med. Chem. 4 (1996), 709-715. Furthermore, the three-dimensional and/or crystallographic structure of inhibitors and the MDR-1 protein of the invention can be used for the design of peptidomimetic drugs (Rose, Biochemistry 35 (1996), 12933-12944; Rutenber, Bioorg. Med. Chem. 4 (1996), 1545-1558).

In summary, the present invention provides methods for identifying and obtaining compounds which can be used in specific doses for the treatment of specific forms of diseases, e.g., cancer the chemotherapy of which is complicated by malfunctions of the MDR-1 gene often resulting in a drug resistant or sensitive phenotype.

In a preferred embodiment of the method of the invention said cell is a cell of or, obtained by the method of the invention or is comprised in the above-described transgenic non-human animal.

In a further embodiment the present invention relates to a method of identifying and obtaining an MDR-1 inhibitor capable of modulating the activity of a molecular variant of the MDR-1 gene or its gene product comprising the steps of
(a) contacting the variant MDR-1 protein of the invention with a first molecule known to be bound by MDR-1 protein to form a first complex of said protein and said first molecule;
(b) contacting said first complex with a compound to be screened; and
(c) measuring whether said compound displaces said first molecule from said first complex.

Advantageously, in said method said measuring step comprises measuring the formation of a second complex of said protein and said inhibitor candidate. Preferably, said measuring step comprises measuring the amount of said first molecule that is not bound to said protein.

In a particularly preferred embodiment of the above-described method of said first molecule is Verapamil, Valspodar, Cyclosporin A or dexniguldipine. Furthermore, it is preferred that in the method of the invention said first molecule is labeled, e.g., with a radioactive or fluorescent label.

In a still further embodiment the present invention relates to a method of diagnosing a disorder related to the presence of a molecular variant MDR-1 gene or susceptibility to such a disorder comprising
(a) determining the presence of a polynucleotide of the invention in a sample from a subject; and/or
(b) determining the presence of a variant form of MDR-1 protein, for example, with the antibody of the invention.

In accordance with this embodiment of the present invention, the method of testing the status of a disorder or susceptibility to such a disorder can be effected by using a polynucleotide or a nucleic acid molecule of the invention, e.g., in the form of a Southern or Northern blot or in situ analysis. Said nucleic acid sequence may hybridize to a coding region of either of the genes or to a non-coding region, e.g. intron. In the case that a complementary sequence is employed in the method of the invention, said nucleic acid molecule can again be used in Northern blots. Additionally, said testing can be done in conjunction with an actual blocking, e.g., of the transcription of the gene and thus is expected to have therapeutic relevance. Furthermore, a primer or oligonucleotide can also be used for hybridizing to one of the above-mentioned MDR-1 genes or corresponding mRNAs. The nucleic acids used for hybridization can, of course, be conveniently labeled by incorporating or attaching, e.g., a radioactive or other marker. Such markers are well known in the art. The labeling of said nucleic acid molecules can be effected by conventional methods. Additionally, the presence or expression of variant MDR-1 genes can be monitored by using a primer pair that specifically hybridizes to either of the corresponding nucleic acid sequences and by carrying out a PCR reaction according to standard procedures. Specific hybridization of the above mentioned probes or primers preferably occurs at stringent hybridization conditions. The term “stringent hybridization conditions” is well known in the art; see, for example, Sambrook et al., “Molecular Cloning, A Laboratory Manual” second ed., CSH Press, Cold Spring Harbor, 1989; “Nucleic Acid Hybridisation, A Practical Approach”, Hames and Higgins eds., IRL Press, Oxford, 1985. Furthermore, the mRNA, cRNA, cDNA or genomic DNA obtained from the subject may be sequenced to identify mutations which may be characteristic fingerprints of mutations the MDR-1 gene. The present invention further comprises methods wherein such a fingerprint may be generated by RFLPs of DNA or RNA obtained from the subject, optionally the DNA or RNA may be amplified prior to analysis, the methods of which are well known in the art. RNA fingerprints may be performed by, for example, digesting an RNA sample obtained from the subject with a suitable RNA-Enzyme, for example RNase T₁, RNase T₂ or the like or a ribozyme and, for example, electrophoretically separating and detecting the RNA fragments as described above.

Further modifications of the above-mentioned embodiment of the invention can be easily devised by the person skilled in the art, without any undue experimentation from this disclosure; see, e.g., the examples. An additional embodiment of the present invention relates to a method wherein said determination is effected by employing an antibody of the invention or fragment thereof. The antibody used in the method of the invention may be labeled with detectable tags such as a histidine flags or a biotin molecule.

In a preferred embodiment of the present invention, the above described methods comprise PCR, ligase chain reaction, restriction digestion, direct sequencing, nucleic acid amplification techniques, hybridization techniques or immunoassays (Sambrook et al., loc. cit. CSH cloning, Harlow and Lane loc. cit. CSH antibodies).

In a preferred embodiment of the method of the present invention said disorder is cancer.

In a further embodiment of the above-described method, a further step comprising administering to the subject a medicament to abolish or alleviate said variations in the MDR-1 gene in accordance with all applications of the method of the invention allows treatment of a given disease before the onset of clinical symptoms due to the phenotype response caused by the MDR-1 gene.

In a preferred embodiment of the method of the invention said medicament are chemotherapeutic agents such as adriamycin, doxorubicin, paclitaxol (taxol) and other MDR-substrates, Ambudkar S V. et al., Annu. Rev. Pharmacol. Toxicol. 39 (1999), 361-398.

In another preferred embodiment of the above-described methods, said method further comprises introducing
(i) a functional and expressible wild type MDR-1 gene or
(ii) a nucleotide acid molecule or vector of the invention into cells.

In this context and as used throughout this specification, “functional” MDR-1 gene means a gene wherein the encoded protein having part or all of the primary structural conformation of the wild type MDR-1 protein, i.e. possessing the biological property of mediating the drug transport through the membrane. This embodiment of the present invention is suited for therapy of cancer, inflammatory diseases, neuronal, CNS diseases or cardiovascular diseases, in particular in humans. Detection of the expression of a variant MDR-1 gene would allow the conclusion that said expression is interrelated to the generation or maintenance of a corresponding phenotype of the disease. Accordingly, a step would be applied to reduce the expression level to low levels or abolish the same. This can be done, for example, by at least partial elimination of the expression of the mutant gene by biological means, for example, by the use of ribozymes, antisense nucleic acid molecules, intracellular antibodies or the above described inhibitors against the variant forms of these MDR-1 proteins. Furthermore, pharmaceutical products may be developed that reduce the expression levels of the corresponding mutant proteins and genes.

In a further embodiment the invention relates to a method for the production of a pharmaceutical composition comprising the steps of any one of the above described methods and synthesizing and/or formulating the compound identified in step (b) or a derivative or homologue thereof in a pharmaceutically acceptable form. The therapeutically useful compounds identified according to the method of the invention may be formulated and administered to a patient as discussed above. For uses and therapeutic doses determined to be appropriate by one skilled in the art see infra.

Furthermore, the present invention relates to a method for the preparation of a pharmaceutical composition comprising the steps of the above-described methods; and formulating a drug or pro-drug in the form suitable for therapeutic application and preventing or ameliorating the disorder of the subject diagnosed in the method of the invention.

Drugs or pro-drugs after their in vivo administration are metabolized in order to be eliminated either by excretion or by metabolism to one or more active or inactive metabolites (Meyer, J. Pharmacokinet. Biopharm. 24 (1996), 449459). Thus, rather than using the actual compound or inhibitor identified and obtained in accordance with the methods of the present invention a corresponding formulation as a pro-drug can be used which is converted into its active in the patient. Precautionary measures that may be taken for the application of pro-drugs and drugs are described in the literature; see, for review, Ozama, J. Toxicol. Sci. 21 (1996), 323-329).

In a preferred embodiment of the method of the present invention said drug or prodrug is a derivative of a medicament as defined hereinbefore.

In a still further embodiment the present invention relates to an inhibitor identified or obtained by the method described hereinbefore. Preferably, the inhibitor binds specifically to the variant MDR-1 protein of the invention. The antibodies, nucleic acid molecules and inhibitors of the present invention preferably have a specificity at least substantially identical to binding specificity of the natural ligand or binding partner of the MDR-1 protein of the invention. An antibody or inhibitor can have a binding affinity to the MDR-1 protein of the invention of at least 10⁵ M⁻¹, preferably higher than 10⁷ M⁻¹ and advantageously up to 10¹⁰ M⁻¹ in case MDR-1 activity should be repressed. Hence, in a preferred embodiment, a suppressive antibody or inhibitor of the invention has an affinity of at least about 10⁻⁷ M, preferably at least about 10⁻⁹ M and most preferably at last about 10⁻¹¹ M.

Furthermore, the present invention relates to the use of an oligo- or polynucleotide for the detection of a polynucleotide of the invention and/or for genotyping of corresponding individual MDR-1 alleles. Preferably, said oligo- or polynucleotide is a polynucleotide or a nucleic acid molecule of the invention described before.

In a particular preferred embodiment said oligonucleotide is about 10 to 100, more preferably 15 to 50 nucleotides in length and comprises the nucleotide sequence of any one of SEQ ID NOS: 1 to 179 or a wild type (“wt”)- or mutated (“mut”)-sequence of the promoter or of an exon of the MDR-1 gene depicted in Table 8 or a complementary sequence of any one of those.

Hence, in a still further embodiment, the present invention relates to a primer or probe consisting of an oligonucleotide as defined above. In this context, the term “consisting of” means that the nucleotide sequence described above and employed for the primer or probe of the invention does not have any further nucleotide sequences of the MDR-1 gene immediately adjacent at its 5′ and/or 3′ end. However, other moieties such as labels, e.g., biotin molecules, histidin flags, antibody fragments, colloidal gold, etc. as well as nucleotide sequences which do not correspond to the MDR-1 gene may be present in the primer and probes of the present invention. Furthermore, it is also possible to use the above described particular nucleotide sequences and to combine them with other nucleotide sequences derived from the MDR-1 gene wherein these additional nucleotide sequences are interspersed with moieties other than nucleic acids or wherein the nucleic acid does not correspond to nucleotide sequences of the MDR-1 gene.

In addition, the present invention relates to the use of an antibody or a substance capable of binding specifically to the gene product of an MDR-1 gene for the detection of the variant MDR-1 protein of the invention, the expression of a molecular variant MDR-1 gene comprising a polynucleotide of the invention and/or for distinguishing MDR-1 alleles comprising a polynucleotide of the invention.

Moreover, the present invention relates to a composition, preferably pharmaceutical composition comprising the antibody, the nucleic acid molecule, the vector or the inhibitor of the present invention, and optionally a pharmaceutically acceptable carrier. These pharmaceutical compositions comprising, e.g., the inhibitor or pharmaceutically acceptable salts thereof may conveniently be administered by any of the routes conventionally used for drug administration, for instance, orally, topically, parenterally or by inhalation. Acceptable salts comprise acetate, methylester, HCl, sulfate, chloride and the like. The compounds may be administered in conventional dosage forms prepared by combining the drugs with standard pharmaceutical carriers according to conventional procedures. These procedures may involve mixing, granulating and compressing or dissolving the ingredients as appropriate to the desired preparation. It will be appreciated that the form and character of the pharmaceutically acceptable character or diluent is dictated by the amount of active ingredient with which it is to be combined, the route of administration and other well-known variables. The carrier(s) must be “acceptable” in the sense of being compatible with the other ingredients of the formulation and not deleterious to the recipient thereof. The pharmaceutical carrier employed may be, for example, either a solid or liquid. Examplary of solid carriers are lactose, terra alba, sucrose, talc, gelatin, agar, pectin, acacia, magnesium stearate, stearic acid and the like. Exemplary of liquid carriers are phosphate buffered saline solution, syrup, oil such as peanut oil and olive oil, water, enulsions, various types of wetting agents, sterile solutions and the like. Similarly, the carrier or diluent may include time delay material well known to the art, such as glyceryl mono-stearate or glyceryl distearate alone or with a wax.

The dosage regimen will be determined by the attending physician and other clinical factors; preferably in accordance with any one of the above described methods. As is well known in the medical arts, dosages for any one patient depends upon many factors, including the patient's size, body surface area, age, the particular compound to be administered, sex, time and route of administration, general health, and other drugs being administered concurrently. Progress can be monitored by periodic assessment.

Furthermore, the use of pharmaceutical compositions which comprise antisense-oligonucleotides which specifically hybridize to RNA encoding mutated versions of a MDR-1 gene according to the invention or which comprise antibodies specifically recognizing mutated MDR-1 protein but not or not substantially the functional wild-type form is conceivable in cases in which the concentration of the mutated form in the cells should be reduced.

Thanks to the present invention the particular drug selection, dosage regimen and corresponding patients to be treated can be determined in accordance with the present invention. The dosing recommendations will be indicated in product labeling by allowing the prescriber to anticipate dose adjustments depending on the considered patient group, with information that avoids prescribing the wrong drug to the wrong patients at the wrong dose.

Furthermore, the present invention relates to a diagnostic composition or kit comprising any one of the afore-described polynucleotides, vectors, host cells, variant MDR-1 proteins, antibodies, inhibitors, nucleic acid molecules or the corresponding vectors of the invention, and optionally suitable means for detection. The kit of the invention may contain further ingredients such as selection markers and components for selective media suitable for the generation of transgenic cells and animals. The kit of the invention may advantageously be used for carrying out a method of the invention and could be, inter alia, employed in a variety of applications, e.g., in the diagnostic field or as research tool. The parts of the kit of the invention can be packaged individually in vials or in combination in containers or multicontainer units. Manufacture of the kit follows preferably standard procedures which are known to the person skilled in the art. The kit or diagnostic compositions may be used for methods for detecting expression of a mutant form of MDR-1 gene in accordance with any one of the above-described methods of the invention, employing, for example, immuno assay techniques such as radioimmunoassay or enzymeimmunoassay or preferably nucleic acid hybridization and/or amplification techniques such as those described herein before and in the examples.

Some genetic changes lead to altered protein conformational states. For example, some variant MDR-1 proteins possess a tertiary structure that renders them far less capable of fascilitating drug transport. Restoring the normal or regulated conformation of mutated proteins is the most elegant and specific means to correct these molecular defects, although it is difficult. Pharmacological manipulations thus may aim at restoration of wild-type conformation of the protein. Thus, the polynucleotides and encoded proteins of the present invention may also be used to design and/or identify molecules which are capable of activating the wild-type function of a MDR-1 gene or protein.

In another embodiment the present invention relates to the use of a drug or prodrug for the preparation of a pharmaceutical composition for the treatment or prevention of a disorder diagnosed by the method described hereinbefore.

Furthermore, the present invention relates to the use of an effective dose of a nucleic acid sequence encoding a functional and expressible wild type MDR-1 protein for the preparation of a pharmaceutical composition for treating, preventing and/or delaying a disorder diagnosed by the method of the invention. A gene encoding a functional and expressible MDR-1 protein can be introduced into the cells which in turn produce the protein of interest. Gene therapy, which is based on introducing therapeutic genes into cells by ex-vivo or in-vivo techniques is one of the most important applications of gene transfer. Suitable vectors and methods for in-vitro or in-vivo gene therapy are described in the literature and are known to the person skilled in the art; see, e.g., Giordano, Nature Medicine 2 (1996), 534-539; Schaper, Circ. Res. 79 (1996), 911-919; Anderson, Science 256 (1992), 808-813; Isner, Lancet 348 (1996), 370-374; Muhlhauser, Circ. Res. 77 (1995), 1077-1086; Wang, Nature Medicine 2 (1996), 714-716; WO94/29469; WO 97/00957 or Schaper, Current Opinion in Biotechnology 7 (1996), 635-640, and references cited therein. The gene may be designed for direct introduction or for introduction via liposomes, or viral vectors (e.g. adenoviral, retroviral) into the cell. Preferably, said cell is a germ line cell, embryonic cell, or egg cell or derived therefrom, most preferably said cell is a stem cell.

As is evident from the above, it is preferred that in the use of the invention the nucleic acid sequence is operatively linked to regulatory elements allowing for the expression and/or targeting of the MDR-1 protein to specific cells. Suitable gene delivery systems that can be employed in accordance with the invention may include liposomes, receptor-mediated delivery systems, naked DNA, and viral vectors such as herpes viruses, retroviruses, adenoviruses, and adeno-associated viruses, among others. Delivery of nucleic acids to a specific site in the body for gene therapy may also be accomplished using a biolistic delivery system, such as that described by Williams (Proc. Natl. Acad. Sci. USA 88 (1991), 2726-2729). Standard methods for transfecting cells with recombinant DNA are well known to those skilled in the art of molecular biology, see, e.g., WO 94/29469; see also supra. Gene therapy may be carried out by directly administering the recombinant DNA molecule or vector of the invention to a patient or by transfecting cells with the polynucleotide or vector of the invention ex vivo and infusing the transfected cells into the patient.

In a preferred embodiment of the uses and methods of the invention, said disorder is cancer or a neuronal, CNS or cardiovascular disease.

As shown in Examples 6 and 8 the polymorphisms identified in accordance with the present invention, especially the single nucleotide polymorphism (SNP)C3435T in exon 26 of the MDR-1 gene are useful as a pharmacogenetic factor that enables the prediction of blood levels of diverse MDR-1 substrates and inducers for improvement of drug safety and efficacy, i.e. to predict and prevent side effects and drug interactions and to increase patient compliance. Such substrates and inducers are, for example, anticonvulsant/antiepileptic drugs, like Phenyloin; cardiac glycosides, like Digoxin; immunosuppressive drugs like Cyclosporin A and FK506; macrolid-antibiotics, like Clarithromycin and Erythromycin; and macrocyclic-antibiotics, like Rifampin. Thus, the present invention also relates to the use of the above described SNPs as a pharmacogenetic factor in accordance with the above. Preferably, the polymorphism is the MDR-1 exon 26 (C3435T) SNP either alone or in conjunction with any other SNP such as those described above.

Further applications of the polymorphisms identified in accordance with the present invention and means and methods that can be used in accordance with the above described embodiments can be found in the prior art, for example, as described in U.S. Pat. No. 5,856,104, wherein the there described means and methods for forensics, Paternity testing, correlation of polymorphisms with phenotypic traits, genetic mapping of phenotypic traits, etc. can be equally applied in accordance with the present invention.

These and other embodiments are disclosed or are obvious from and encompassed by the description and examples of the present invention. Further literature concerning any one of the methods, uses and compounds to be employed in accordance with the present invention may be retrieved from public libraries, using for example electronic devices. For example the public database “Medline” may be utilized which is available on Internet, e.g. under http://www.ncbi.nlm.nih.gov/PubMed/medline.html. Further databases and addresses, such as http://www.ncbi.nim.nih.gov/, http://www.infobiogen.fr/, http://www.fmi.ch/biology/research_tools.html, http://www.tigr.org/, are known to the person skilled in the art and can also be obtained using, e.g., http://www.lycos.com. An overview of patent information in biotechnology and a survey of relevant sources of patent information useful for retrospective searching and for current awareness is given in Berks, TIBTECH 12 (1994), 352-364.

The pharmaceutical and diagnostic compositions, uses, methods of the invention can be used for the diagnosis and treatment of all kinds of diseases hitherto unknown as being related to or dependent on variant MDR-1 genes. The compositions, methods and uses of the present invention may be desirably employed in humans, although animal treatment is also encompassed by the methods and uses described herein.

### BRIEF DESCRIPTION OF THE FIGURES

FIG. 1: Gel of selected PCR fragments, before and after purification. Agarose (Appli Chem, Darmstadt) gel electrophoresis (1.5% Agarose gel) of MDR-1 PCR fragments before (A) and after (B) the purification step. M: molecular weight markers, 1-28: PCR fragments containing the sequences of exons 1-28 of the human MDR-1 gene, including relevant sequences that are flanking these exons.

FIG. 2: Examples for homozygous and heterozygous MDR-1 alleles. The sequences of PCR fragments containing the sequences of exons 1-28 of the human MDR-1 gene, including relevant sequences that are flanking these exons, were determined by automated sequencing using the ABI Dyeterminator techniques. Heterozygous and homozygous deviations from the published MDR-1 sequence can be detected directly in the DNA sequence profiles.

FIG. 3: Examples for diagnosis of homozygous and heterozygous MDR-1 alleles. Agarose (AppliChem, Darmstadt) gelelectrophoresis (1.5% Agarose gel) of the allele specific PCR fragments of exon 2 (261 bp) and exon 5 (180 bp).

The invention will now be described by reference to the following biological examples which are merely illustrative and are not to be construed as a limitation of the scope of the present invention.

### EXAMPLES

### Example 1

### Isolation of Genomic DNA from Human Blood, Generation and Purification of MDR-1 Gene Fragments

Genomic DNA was obtained by standard ion exchange chromatography techniques (Quiagen kits for isolation of genomic DNA from blood). Blood from all the individuals that were tested (volunteers from the department of Pharmacology at the Charitee Berlin) was obtained under consideration of all legal, ethical and medical and bureaucratical requirement of the Charitee Clinicum in Berlin, Germany.

Specific oligonucleotide primers, 2 for each fragment, were applied to obtain by polymerase chain reaction (PCR) defined DNA fragments containing specific parts of the human MDR-1 gene. These specific oligonucleotide primers were designed to bind to sequences upstream and downstream of the various exons of the MDR-1 gene. The resulting DNA fragments were to encode not only exon sequences, but also some intron sequences at the exon-intron boundaries. Such intronic sequences close to the exons are known to be important for correct processing and subsequent expression of the protein encoding mRNA, a process known as “splicing”. Oligonucleotide primer pairs that were optimized for each of the 28 exons of the human MDR-1 gene, synthesized and purified by affinity chromatography (OPC cartridges). The sequence for each of primer is listed in Table 1.

Polymerase chain reactions were performed under conditions that were optimized for each of the fragments that cover the 28 exons of the human MDR-1 gene as well as the core promoter and enhancer region. PCRs were carried out for all exons in a reaction volume of 25 μl. 50 ng DNA template was added to standard PCR buffer containing 1,5 mM MgCl₂ (Quiagen, Hilden), 50 μM dNTP's (Quiagen, Hilden), 25 pMol each primer (Metabion, Munich) and 0,625 U Taq polymerase (Quiagen, Hilden). All PCRs were performed on a Perkin Elmer thermocycler (model 9700) with an initial denaturation step of 2 min at 94° C. and 36 amplification cycles of denaturation 94° C. for 45 sec, primer annealing depending on the primer's melting temperature (PCR conditions: A-H) for 45 sec, and 45 sec for 72° C. followed by a final extension of 72° C. for 5 min. For the single PCR conditions A-H the following annealing temperatures were applied: A: 53° C.; B: 56° C.; C: 55° C. D: 57,5° C.; E: 58° C.; F: 59° C.; G: 54° C.; H: 60° C.

PCRs were carried out for all fragments (promoter and enhancer) in a reaction volume of 50 μl. 50 ng DNA template (exceptions: 10 ng for promoter fragments 1-3) was added to standard PCR buffer containing 1,5 mM MgCl2 (Quiagen, Hilden), 200 μM dNTP's (Quiagen, Hilden), 30 pMol each primer (Metabion, Munich; exception: 20 pMol for enhancer fragment 1) and 1 U Taq polymerase (Quiagen, Hilden). All PCRs were performed on a Perkin Elmer thermocycler (model 9700) with an initial denaturation step of 3 min at 94° C. and different amplification cycles (30 for promoter fragment 2+4 and enhancer fragment 1; 31 for promoter fragment 3; 32 for promoter fragment 1 and enhancer fragment 2) of denaturation 94° C. for 30 sec, primer annealing depending on the primer's melting temperature (PCR conditions: A and B) for 30 sec, and 30 sec for 72° C. followed by a final extension of 72° C. for 2 min. For the single PCR conditions A and B the following annealing temperatures were applied: A: 58° C.; B: 56° C.

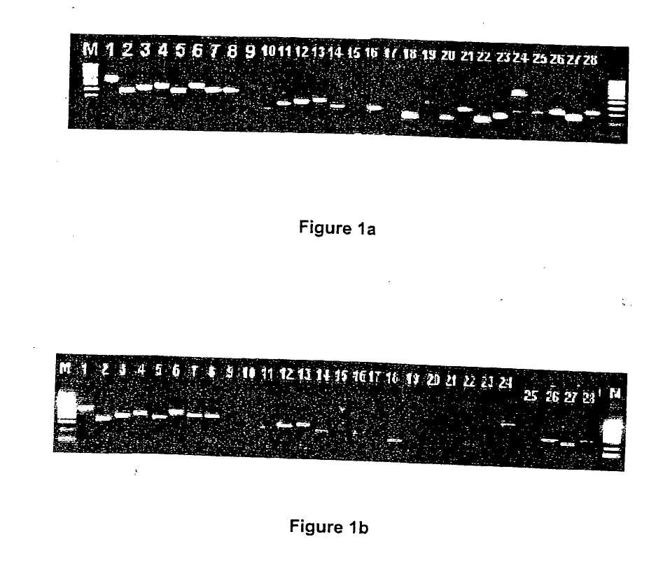

The optimized PCR-conditions and the resulting size of the desired and obtained fragments are listed in Table 1. Examples of the resulting MDR-1 gene fragments that were used for further analysis of the individual genotype are presented in FIG. 1.

The defined DNA fragments containing specific parts of the human MDR-1 gene, exon sequences as well as some intron sequences at the exon-intron boundaries were processed to remove nonincorporated nucleotides and buffer components that otherwise might interfere with the subsequent determination of the individual MDR-1 genotype by direct DNA sequencing. For this purification, standard ion exchange chromatography techniques were used (Quiagen kits for PCR fragment purification). For all of the fragments, sufficient yields of purified fragments, suitable for direct DNA sequence analyses, were obtained. Examples of purified MDR-1 gene fragments that were used for direct sequence analysis of the individual MDR-1 genotype are presented in FIG. 1.

### Example 2

### Identification of Different MDR-1 Gene Alleles by Sequence Determination in Various Individuals

For the sequence analysis of relevant regions of the human MDR-1 gene from many different individuals, PCR amplification of the relevant regions of the MDR-1 gene were carried out (see Tab.1) and the purified PCR products subsequently sequenced with established methods (ABI dyeterminator cycle sequencing). A very important parameter that was needed to consider using this approach was that each normal human individual harbors two MDR-1 gene copies. Because of this diploidy (of autosomal genes, and MDR-1 is autosomally encoded), great care had to be taken in the evaluation of the sequences to be able to identify unambiguously not only homozygous sequence variations but also heterozygous variations. Because of that, it was never relied on only one determined sequence, but always obtained at least two sequences from each defined MDR-1 gene fragment from each individual, by sequencing both opposite DNA strands.

For the initial evaluation of MDR-1 variations in the human population, sequence analysis of the relevant regions, including all exons, of the human MDR-1 gene was carried out from the genomic DNA from 24 different individuals. This number of individual samples was then extended for selected MDR-1 gene fragments, some of which have been analyzed from 127 individuals. The sequences were manually inspected for the occurrence of DNA sequences that were deviant from the published MDR-1 sequences, which are considered as “wildtype” sequences in all of this work. Because population genetics enables a calculation of the expected frequency of homozygous vs. heterozygous alleles of a defined gene (Hardy Weinberg formula, p e2+2pq+q e2=1), it was also possible to confirm the predicted (with that formula) distribution of homozygous vs. heterozygous alleles and deviations with the experimental findings. This serves as internal control and confirmation that a detected sequence deviation indeed represents a novel allele.

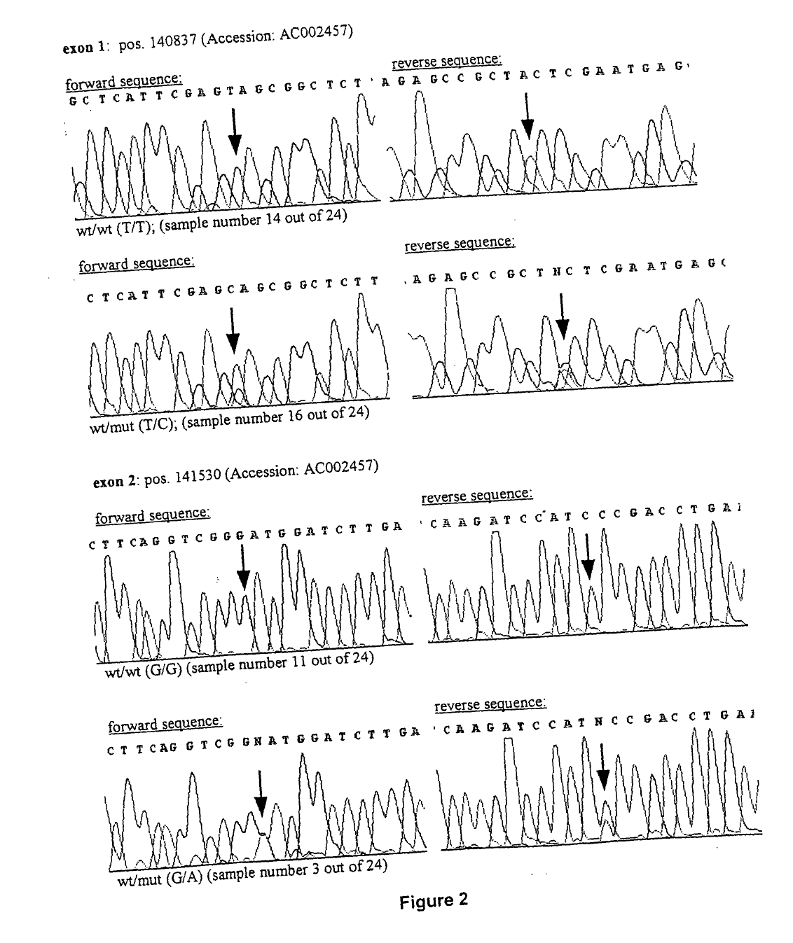

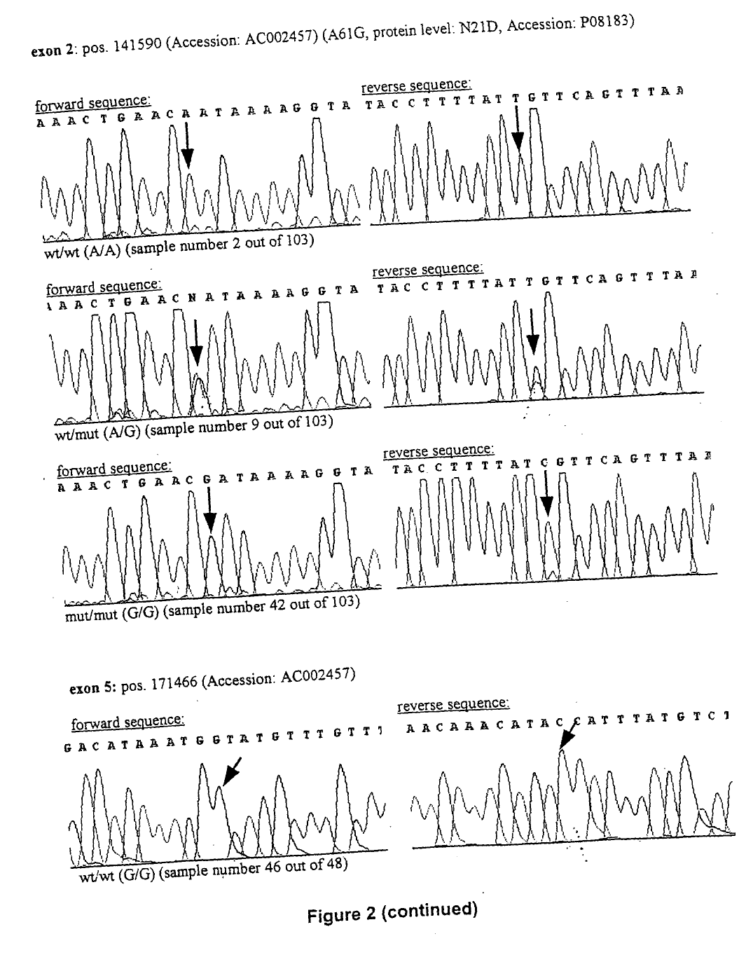

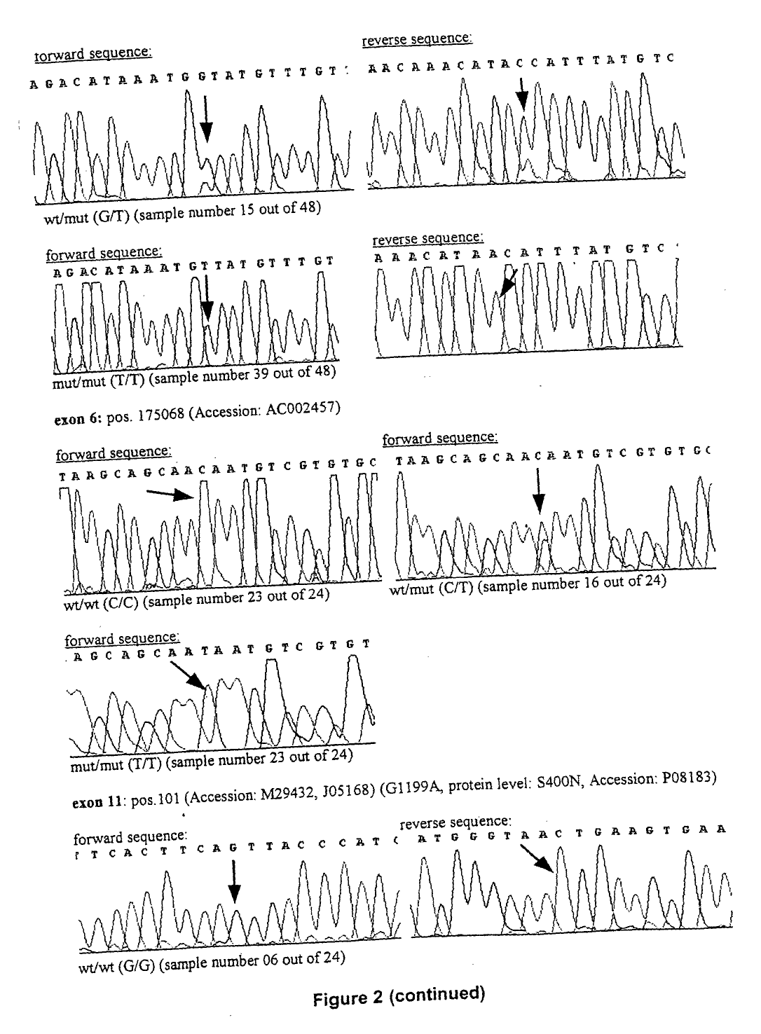

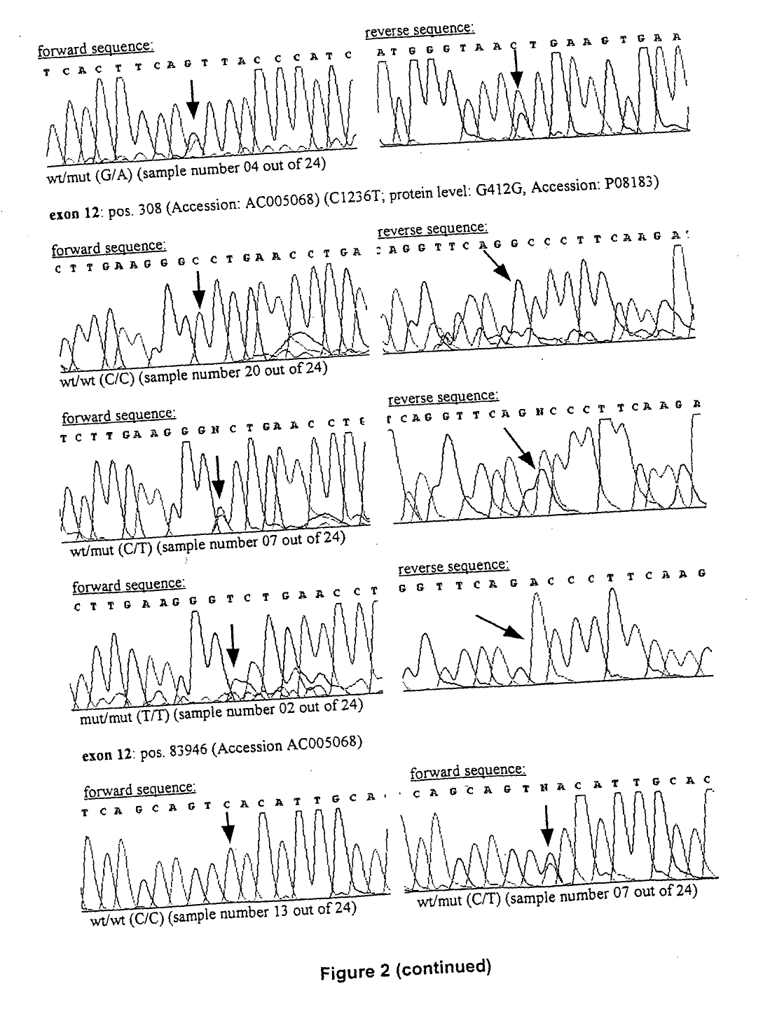

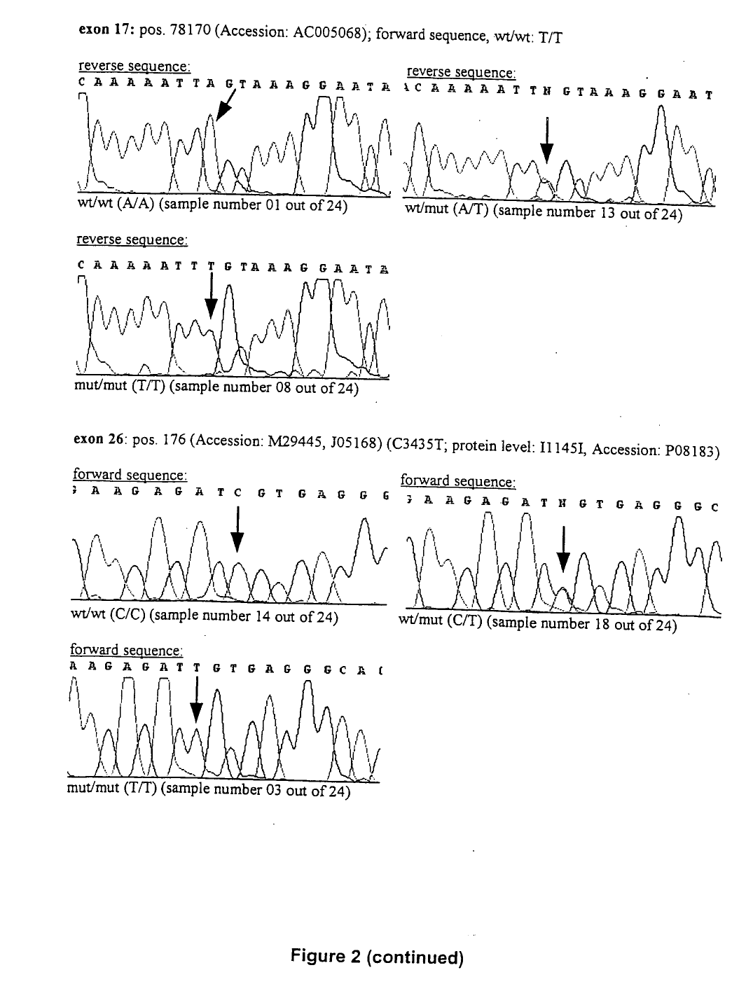

Several novel MDR-1 sequence variations were discovered and experimentally confirmed using this approach which are shown in FIG. 2. 8 polymorphisms appear in intron sequences close flanking the exons 5, 6, 12 and 17 (SEQ ID NOs: 91, 154 and 160 for exon 5), (SEQ ID NOs: 101 and 166 for exon 6), (SEQ ID NO: 116 for exon 12) and (SEQ ID NOs: 119 and 172 for exon 17). 7 polymorphisms were found in the coding region, 2 in the exons 2 and 26, and one each in exons 5, 11 and 12 and one in noncoding exon 1 (SEQ ID NOs: 79 and 85 (for exon 2), 122 and 178 (for exon 26), 97 (for exon 5), 106 (for exon 11), 112 (for exon 12) and 73 (for exon 1), respectively). 3 variations result in changes in the amino acid sequence of the MDR-1 protein (SEQ ID NOs: 85 (for N21D), 97 (for F103S) and 106 (for S400N), respectively). Their changes will alter the MDR protein. One change that does not alter the protein is located directly before the ATG translational start codon (SEQ ID NO: 79). It is well known that this position is very important for the levels of expression of proteins by controlling the effectiveness of translation. Further polymorphisms do not change the amino acid composition of MDR-1, but they still are useful tools for MDR-1 genotyping because each of these variations define a novel MDR-1 allele. It is known that the expression of MDR-1 varies greatly between different individuals, and one very likely explanation for this variability in expression levels is allelic differences in the region directly in and surrounding the MDR-1 gene. Thus all novel and defined MDR-1 alleles serve as markers for the determination of the MDR-1 gene status in patients The importance of this MDR-1 genotyping for the diagnosis and therapy of diseases is well known to experts in the field, and it has also been explained in detail above in the introductory chapter.

The exact positions and further details of the novel MDR-1 alleles, including the exact novel sequence and sequence deviation, and the homozygous vs. heterozygous distribution of the allele in the population are listed in Table 2. The expected frequency for homozygotes of the variant allele were calculated on the basis of the Hardy-Weinberg distribution. The deviant base in the sequence is bold and underlined. FIG. 2 shows examples of the discovery and appearance of novel variants in DNA samples from homozygous or heterozygous individuals.

### Example 3

### Methods for Specific Detection and Diagnosis of MDR-1 Alleles

Methods to detect the various MDR-1 alleles that have been identified utilize the principle that specific sequence differences can be translated into reagents for allele differentiation. These reagents provide the necessary backbone for the development of diagnostic tests. Examples for such reagents include—but are not limited to—oligonucleotides that deviate from the wildtype MDR-1 sequence in the newly identified base substitution. Frequently, the principles of diagnostic tests for the determination of the individual MDR-1 gene status include—but are not limited to-differences in the hybridization efficiencies of such reagents to the various MDR-1 alleles. In addition, differences in the efficacy of such reagents in, or as different substrates for, enzymatic reactions, e.g. ligases or polymerases or restriction enzymes can be applied. The principles of these tests are well known to experts in the field. Examples are PCR- and LCR techniques, Chip-hybridizations or MALDI-TOF analyses. Such techniques are described in the prior art, e.g., PCR technique: Newton, (1994) PCR, BIOS Scientific Publishers, Oxford; LCR-technique: Shimer, Ligase chain reaction. Methods Mol. Biol. 46 (1995), 269-278; Chip hybridization: Ramsay, DNA chips: State-of-the art. Natrue Biotechnology 16 (1998), 40-44; and MALDI-TOF analysis: Ross, High level multiplex genotyping by MALDI-TOF mass spectrometry, Nature Biotechnology 16 (1998), 1347-¹³⁵¹. Other test principles are based on the application of reagents that specifically recognize the MDR-1 variant as translated expressed protein. Examples are allele-specific antibodies, peptides, substrate analogs, inhibitors, or other substances which bind to (and in some instances may also modify the action of) the various MDR-1 protein forms that are encoded by the new MDR-1 alleles. The examples that are presented here, to demonstrate the principles of diagnostic tests with reagents derived from the novel nucleotide substitutions defined in this application, are based on PCR-methods. It is obvious that, applying the described specific reagents, any of the other methods will also work for the differentiation of MDR-1 alleles.

### Example 4

### Diagnosis of MDR-1 Alleles by Specific PCR

Allele-specific PCR is a technique well known to experts in the field that allows the differentiation of alleles of genes by the application of the polymerase chain reaction with reagents (primer combinations) that are specifically designed for the detection of single allele sequences. The main component of such tests, and the only reagent that provides the specificity of such tests, are oligonucleotides that are designed to contain sequences that specifically distinguish different alleles of genes.

In this example, specific oligonucleotides were designed that can distinguish different MDR-1 alleles because of their differential hybridization efficacy to different alleles and because of their varying ability to serve as substrates for enzymatic reactions (the enzyme in this example being a thermostable polymerase). The reagents that were specifically designed and able to detect the presence and/or absence of the newly defined mdr-1 alleles in individual humans are listed as specific primer combinations for each new allele in Table 3. The design of these reagents bases on the newly discovered nucleotide sequences and base substitutions in the human MDR-1 gene, which are presented in example 2 and listed in Table 2 and FIG. 2. In addition to the design of specific reagents, diagnostic test that are based upon the principle of polymerase chain reaction needs optimization of test conditions, i.e. optimized PCR-conditions. The result of test is in this case given as presence or absence of specific DNA fragments obtained using genomic DNA from individual humans as testable ingredient (template). The preparation of the genomic DNA from the blood of individuals is described in example 1.

PCRs were carried out for all fragments in a reaction volume of 20 μl. 50 ng DNA template was added to standard PCR buffer (Qiagen, Hilden) containing 1,5 mM MgCl₂, 250 μM dNTP's (Qiagen, Hilden), 1× Q-solution (Qiagen, Hilden), 20 pMol each primer (Metabion, Munich; specific wt primer+common primer and specific mut primer+common primer) and 1 U Taq polymerase (Qiagen, Hilden). All PCRs were performed on a Perkin Elmer thermocycler (model 9700) with an initial denaturation step of 3 min at 95° C. and 30 amplification cycles of denaturation 94° C. for 30 sec, primer annealing depending on the primer's melting temperature (PCR conditions: A-E) for 30 sec, and 30 sec for 72° C. followed by a final extension of 72° C. for 8 min. For the single PCR conditions A-E the following annealing temperatures were applied: A: 54° C.; B: 58° C.; C: 50° C.; D: 61° C.; E: 53° C.

The deviant base in the respective specific primer sequence is underlined and in a bold style. The presence or absence of specific DNA fragments in this assay translates in presence or absence of the tested allele.

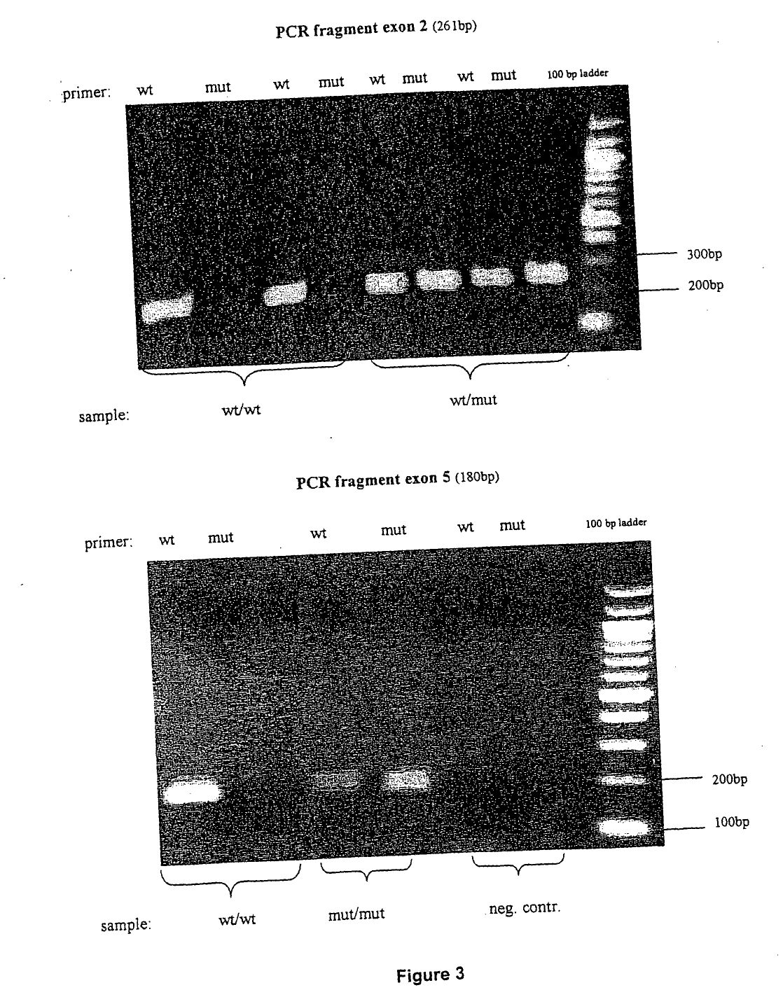

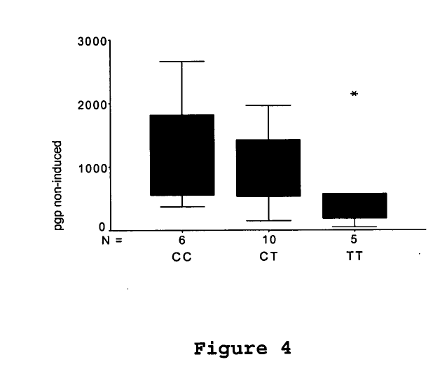

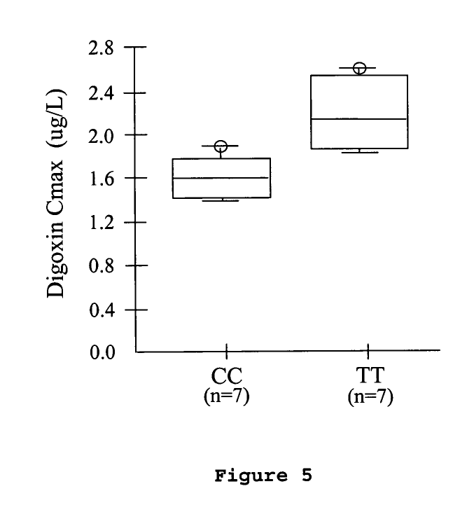

Examples for such readouts, as results for the MDR-1 allele detection diagnosis, are shown FIG. 3. It is obvious from these examples (Tab.3, FIG. 3), that these tests are suitable to differentiate the presence of the analyzed MDR-1 alleles in humans. Homozygous as well as heterozygous, frequent as well as rare alleles of the MDR-1 gene can be detected. The specificity of these tests relies solely, and totally depends, on the specific oligonucleotide reagents that were applied. The design of these reagents in turn was dependent on the sequence information of the discovered MDR-1 variants and novel alleles, that are presented in example 2 and Table 2.

### Example 5

### Diagnosis and Correlation of Different MDR-1 Polymorphisms with Expression Levels and In Vivo Activity of MDR-1 in Patients

To identify potential direct correlations of MDR-1 polymorphisms with clinical relevant phenotypes in humans, probands from a study at the Dr. Margarete Fischer-Bosch-Institut for Clinical Pharmacology in Stuttgart, were subjected to the determination of MDR-1 polymorphisms as described in examples 2-4. The expression levels of MDR-1 in the colon and liver of these patients was also estimated by established immunohistochemical detection of the MDR-1 protein. In the proband population, in addition to measurements of the expression levels of MDR-1 in the colon, measurements of MDR-1 upon induction of the gene by rifampicine were performed. Also, the in vivo activity of MDR-1 under noninduced and rifampicine induced conditions was determined by measuring the blood concentrations of orally administered digoxin (1 mg), which is a known MDR-1 substrate and whose blood concentration also depends on the MDR-1 activity in the colon. The results of the MDR-1 measurements, rifampicine induction experiments and digoxin-experiments, as well as results from the MDR-1 polymorphism detection analysis in the proband population show correlations between MDR-1 gene expression and MDR-1 in vivo activity with certain polymorphisms.

MDR-1 Protein Levels:

As shown in table 4, a T/C polymorphism at position 176 in Acc.#M29445/J05168 in exon 26 correlates with the expression levels of MDR-1. Presence of the T allele at this position indicates weaker MDR-1 expression levels compared to samples which have only the corresponding homozygous C-allele. The mean of the rifampicin-induced MDR-1 levels of the C-allele population is much higher as that of the T-population (924 vs 587 relative units). In total agreement with that, a proband homozygous for the T-allele had the lowest detectable uninduced and induced MDR-1 level while a proband homozygous for the C allele displayed the highest level of all probands tested. The difference of induced MDR-1 expression levels between these individuals was 9-fold.

MDR-1 In Vivo Activity:

Table 5 shows the results of the measurements of the in vivo activity of MDR-1 under noninduced and rifampicine induced conditions. This was done by measuring the blood concentrations of orally administered digoxin which is a known MDR-1 substrate and whose blood concentration also depends on the MDR-1 activity in the colon. Consistant with the observation that the polymorphism at position 176 in Acc.#M29445/J05168 in exon 26 T/C correlates with the expression levels of MDR-1, a correlation of this polymorphism was observed with digoxin blood levels, which in turn reflects the MDR-1 protein activity in vivo. The probands that harbor the T allele (correlates with weaker MDR-1 expression, see Tab. 4), contain higher blood levels of digoxin compared to samples which have only the corresponding homozygous C-allele. The reason for that is that the uptake of MDR-1 substrates such as digoxin from the colon to the blood appears to be more effective in humans with lower MDR-1 expression. This is totally consistent with the function of MDR-1 in the colon, i.e. re-transport and elimination of substrates from the uptaking cells into the lumen of the colon. The mean of the non-induced as well as rifampicin-induced digoxin concentration in the blood (correlates invers to MDR-1 activity) of the C-allele population are consistantly lower than those of the T-population (63.9 vs. 44.9 and 45 vs. 28.6 Dig AUC induced). In total agreement with that, a proband with the homozygous T allele had the highest detectable digoxin concentration in the blood after rifampicine induction (57.3 Dig.AUC) and a proband homozygous for the C-allele displayed the lowest level of all probands (12.3 Dig.AUC). The difference of the digoxin blood levels between these individuals was more than 4-fold.

MDR-1 in a Patient Population:

The results of our analysis of the correlation of MDR-1 expression levels, MDR-1 protein activity and MDR-1 polymorphism detection analysis are further corrobated by an analysis of the MDR-1 expression and MDR-1 genotyping of various patients from the Dr. Margarete Fischer-Bosch-Institut for Clinical Pharmacology in Stuttgart. Immunohistology was performed on the various patient tissue samples, particularly colon and liver, and they were compared to each other to allow a relative comparison of the MDR-protein between these samples. Within each set of experiments, patient samples were ranked according to their MDR-1 staining intensity, i.e. 1^{st} rank equals highest MDR-intensity and last rank lowest MDR-1 intensity.

The correlation of this ranking analysis with the MDR-1 genotype shows that the T allele at the polymorphism at position 176 in Acc.#M29445/J05168 in exon 26 correlates with lower expression of the MDR-1 gene when compared to patients which carry homozygous the C allele at this position. In this analysis, two other polymorphisms showed some correlation with MDR-1 expression: A homozygous T genotype at position 171466 in AC002457 (intron 4) may correlate with high expression and a polymorphism (GA) at position 101 in exon 11 (M29432/J05168) may correlate with low expression.

### Example 6

### Validation of the Genotype/Phenotype Correlation of the Exon 26 (C3435T) Polymorphism with Extended Sample Numbers

To further validate the correlation of the single nucleotide polymorphism (SNP) T/C at position 176 in ACC.# M29445/J05168 described in the previous examples and now also referred to as MDR-1 exon 26 SNP C3435T, (position correspond to MDR-1 cDNA GenBank accession no. AF016535, Codon TTC exon 10, F335, is missing in that sequence), with the first base of the ATG start codon set to 1) with the levels of intestinal MDR-1 expression (first results shown in Example 5), additional volunteers of a further experimental study at the Dr. Margarete Fischer-Bosch-Institute for Clinical Pharmacology in Stuttgart were analysed. The expression levels of MDR-1 in the intestine of these volunteers and patients had been determined by quantitative immunohistochemistry and Western blots of biopsies and enterocyte preparations of the duodenum. To assure that this analysis reflect the specific PGP expression in intestinal enterocytes, an additional marker protein that is expressed in enterocytes, villin, was simultanously analyzed. The results of this analysis are shown in FIG. 4. The T/T genotype is associated with significant lower MDR-1 expression levels compared to the C/C genotype. Individuals with a C/T genotype show an intermediate phenotype.

For a further validation of the correlation of MDR-1 genotype with intestinal digoxin uptake, additional volunteers of another clinical study at the University Medical Center, Charite in Berlin that addresses blood levels of digoxin after oral application (without rifampin induction and PGP protein determination, Johne et al. (1999), Clin.

Pharmacol. Thr. 66, 338-345) were evaluated for their MDR-1 genotype in exon 26. In this study, maximum plasma concentrations (Cₘₐₓ) were evaluated during steady state conditions of digoxin. This pharmacokinetic parameter especially reveals differences in the absorption of digoxin between the different groups. FIG. 5 shows a comparison of digoxin Cₘₐₓ of 7 volunteers that carried homozygously the T/T allele and 7 volunteers with the homozygous C/C genotype in exon 26. Volunteers homozygous for the T-allele show significant higher levels of digoxin compared to volunteers with a C/C genotype. The mean difference of 38% in digoxin Cₘₐₓ between the groups is statistically significant (p=0.006, Mann Whitney U 2 sample test) and reflects the impact of this polymorphism on digoxin pharmacokinetics.

[image]

[image]

### Example 7

### Identification of New MDR-1 Polymorphisms by Sequence Analysis of a Large Collection of Various Individuals

An extended search for SNPs in the human MDR-1 gene revealed in addition to the different novel MDR-1 polymorphisms numerous further new polymorphisms in the MDR-1 gene which are listed in Table 8. Within the new screen the number of individual samples was extended for all MDR-1 exons, as well as for the MDR-1 promoter fragments, some of which have been analyzed from 236 individuals.

It is possible that in addition to the MDR-1 exon 26 (C3435T) SNP that can be used to predict PGP expression, other more rare polymorphisms in regions of the MDR-1 gene have also some affect on expression. E.g. promoter polymorphisms and protein changing SNPs are very likely to have an additional effect on MDR-1 expression and activity. Futhermore, al these novel polymorphisms can be utilized to generate an exact individual MDR-1 genotype—i.e. allele composition—which may be unique for individuals and thus very useful to predict individual MDR-1 dependent drug response.

The more polymorphisms are known in the human MDR-1 gene, the more complete and thus more useful will an individual MDR-1 genotype description be. The identification of these 32 new MDR-1 polymorphisms is an further important step towards achieving the goal of establishing many different MDR-1 genotypes that predict outcome and side effects of drug therapy.

### Example 8

### Determination of the MDR-1 Exon 26 (C3435T) Polymorphism as a Pharmacogenetic Factor that Influences Drug Levels in Combination with Other Pharmacogenetic Factors

The anticonvulsant drug Phenytoin is commonly used in the therapy of epilepsia, acute and chronic suppression of ventricular arrhythmias and in digitalis intoxication. The narrow therapeutic range with a number of severe side effects in combination with a nonlinear pharmacokinetic (i.e. overproportional increase of plasma levels in response to dosage elevation) make Phenyloin treatment challenging and suitable parameters to predict plasma levels from a given dose highly desirable in order to improve therapeutic outcome and to prevent side effects.

It is known that the polymorphic enzymes 2C9 and 2C19 have an effect on the metabolism of Phenyloin (Mamiya et al. 1998, Epilepsia December;39(12):1317-23), and it has been shown that 2C9 defects can lead to abnormal blood levels that may cause side effects or drug inefficacy (Aynacioglu et al. 1999, Br J Clin Pharmacol. September;48(3):409-15). However, it is also clear that 2C9 genotyping does not permit to make exact bloodlevel predictions from given dose. Even in 2C9 genotyped individuals, compensated for the respective enzyme genotype, blood levels vary significantly.

Table 6 shows that MDR-1 exon 26 (C3435T) SNP plays—in addition to 2C9—a clear role in phenyloin blood levels, and MDR-1 genotyping for this SNP permits a more accurate correlation between phenyloin dose, genotype and blood levels.

Within 2C9/19 enzyme genotyped groups, variation of levels can be explained by the MDR-1 genotype, particularly in the groups of 2C9/C19 poor metabolizers, which already show increased blood levels. Here MDR-1 genotyping is able to identify a subgroup of patients who is at increased risk to exhibit extraordinary high phenyloin blood levels: Poor metabolizers which have the MDR-1 T/T genotype. These patients have an increased risk to encounter overdose related adverse drug effects. For example, within a group of 100 patients which received phenyloin, a 2C9 deficient patient with the low PGP (T/T) genotype showed the highest blood concentration, which was about twofold increased compared to the ,,normal” population. The correlation between Cyp 2C9 genotype and Phenyloin plasma levels is statistically significant, but the significance increases by taking into account the MDR-1 T/T genotype as a covariate (p<0.001, ANCOVA).

TABLE 6
Dependence of Phenytoin levels on pharmacogenetic components

| MDR-1 | | |
|---|---|---|
| genotype | CC and CT | TT |
| intestinal PGP | high/medium | low |
| Phenytoin blood | | |
| levels: | | |
| 2C9 | normal | normal |
| metabolizers | | |
| 2C9 weak | high | VERY HIGH |
| and/or deficient | | |
| metabolizers | | |

TABLE 1

| PCR | | | PCR | fragment |
|---|---|---|---|---|
| fragment name | PCR primer position | Primer sequence | conditions | size |
| Exons: | for exons 1-7: | | | |
| | (Accession: AC002457) | | | |
| ex.1 | 140510-140529 | “Forward: 5′CCC,TTA,ACT,ACG,TCC,TGT,AG 3′” | E | 572 bp |
| | 141081-141062 | “Reverse: 5′GAG,GAC,TTC,ACA,CTA,TTC,AC 3′” | | |
| ex.2 | 141423-141442 | “Forward: 5′TCT,TAC,TGC,TCT,CTG,GCC,TTC 3′” | C | 347 bp |
| | 141770-141751 | “Reverse: 5′CTC,AGC,CAA,CAA,ACT,TCT,GC 3′” | | |
| ex.3 | 145681-145700 | “Forward: 5′CAC,TCA,GTG,ATA,ACC,ACG,TA 3′” | D | 385 bp |
| | 146066-146047 | “Reverse: 5′GCA,TCT,CCA,TTA,ACA,TAC,CC 3′ | | |
| ex.4 | 155899-155918 | “Forward: 5′GGG,TGT,CTT,GGA,CTA,GGT,TG 3′” | D | 422 bp |
| | 156320-156301 | “Reverse: 5′TGC,CTC,CTA,CAG,GAC′UAA,AC 3′ | | |
| ex.5 | 171308-171327 | “Forward: 5′CAC,ACA,GTC,AGC,AGA,GAA,GT 3′” | G | 353 bp |
| | 171660-171639 | “Reverse: 5′ACT,ATC,AAG,AGT,ATT,GTT,CTC,C 3′ | | |
| ex.6 | 174661-174680 | “Forward: 5′GGA,ATG,AGT,GGT,CTC,TTT,GG 3′” | E | 442 bp |
| | 175102-175083 | “Reverse: 5′AAG,GCA,CTG,GGA,ACA,AAA,GG 3′” | | |
| ex.7 | 175322-175342 | “Forward: 5′TCC,TAG,PAG,AAA,CTT,CTA,CCC 3′” | E | 390 bp |
| | 175711-175693 | “Reverse: 5′TTC,CGT,AGG,GTG,AGA,GCA,G 3′” | | |
| | for exons 8-28: | | | |
| | (Accession: AC005068) | | | |
| ex.8 | 95327-95307 | “Forward: 5′CAG,ATT,TTG,CTC,TAC,ACA,TGC 3′” | G | 391 bp |
| | 94937-94959 | “Reverse: 5′ATT,AGT,TAT,CCT,GTA,ATA,CAT,CC 3′” | | |
| ex.9 | 87858-87837 | “Forward: 5′CAT,GTA,TAT,CAC,AGG,ACT,GAA,C 3′” | A | 406 bp |
| | 87453-87474 | “Reverse: 5′CTA,GTA,GTG,CAT,ATG,TCT,GTA,G 3′” | | |
| ex.10 | 84881-84861 | “Forward: 5′GTG,ACA,GAA,TGA,GAA,CCT,GTC 3′” | B | 422 bp |
| | 84460-84480 | “Reverse: 5′TGG,AGA,GCT,GGA,TAA,AGT,GAC 3′” | | |
| ex.11 | 84415-84390 | “Forward: 5′AAA,TTG,ATC,TGT,TAG,AAG,CCA,AG 3′” | A | 228 bp |
| | 84184-84206 | “Reverse: 5′ACT,AGG,TTT,AAA,TAT,ACA,TGC,AC 3′” | | |
| ex.12 | 84183-84163 | “Forward: 5′GAA,CAG,TCA,GTT,CCT,ATA,TCC 3′” | D | 260 bp |
| | 83919-83939 | “Reverse: 5′GGG,CAA,CAT,CAG,AAA,GAT,GTG 3′” | | |
| ex.13 | 83939-83919 | “Forward: 5′CAC,ATC,TTT,CTG,ATG,TTG,CCC 3′” | H | 295 bp |
| | 83644-83662 | “Reverse: 5′AAG,GGC,AAA,GGG,CAA,GGA,C 3′” | | |
| ex.14 | 83354-83334 | “Forward: 5′TGG,GTT,TTC,TGT,CGT,AGA,AAT 3 ” | A | 214 bP |
| | 83141-83160 | “Reverse: 5′GTT,GGT,TTG,AAC,TAA,GCC,TC 3′” | | |
| ex.15 | 79861-79841 | “Forward: 5′AAA,TTT,CTC,TCT,CTT,TAG,GCC 3′” | G | 199 bp |
| | 79663-79684 | “Reverse: 5′TTC,TGA,AGT,TAA,ACT,ATA,CCT,G 3′” | | |
| ex.16 | 78834-78812 | “Forward: 5′TTC,TTA,TTT,ATT,TTA,GAC,AGC,AG 3′” | B | 209 bp |
| | 78626-78644 | “Reverse: 5′GCA,TCT,CCC,TTC,ATA,CCA,G 3′” | | |
| ex.17 | 78207-78185 | “Forward: 5′CAT,TGA,TAA,GGA,ATA,AGG,ATA,GG 3′” | B | 427 bp |
| | 77781-77808 | “Reverse: 5′CCT,ATC,ACA,AAC,CAG,ACT,GC 3′” | | |
| ex.18 | 75482-75382 | “Forward: 5′ATT,TCC,AGC,GTA,CTA,AGG,CTC 3′” | A | 358 bp |
| | 75045-75065 | “Reverse: 5′ACT,TGG,CTG,TTA,GTA,GCC,ATG 3′” | | |
| ex.19 | 73301-73281 | “Forward: 5′GTC,ACA,GAA,ACA,TAG,CAA,GCC 3′” | B | 329 bp |
| | 72973-72993 | “Reverse: 5′CAA,CCA,ATA,TAG,GAG,CTA,GGC 3′” | | |
| ex.20 | 70482-70161 | “Forward: 5′ACA,TCC,TGA,GTA,CTA,AAT,GCA,G 3′” | C | 351 bp |
| | 70132-70154 | “Reverse: 5′AAA,GCA,TGT,GAT,ATA,TTC,GTA,GG 3′” | | |
| ex.21 | 65342-65319 | “Forward: 5′TTT,CTC,TAA,TTT,GTT,TTG,TTT,TGC 3′” | B | 245 bp |
| | 65098-65110 | “Reverse: 5′TTT,AGT,TTG,ACT,CAC,CTT,CCC 3′” | | |
| ex.22 | 54877-54857 | “Forward: 5′CCT,TCC,AGA,TCC,ATA,ACT,CAG 3′” | C | 372 bp |
| | 54506-54527 | “Reverse: 5′TAC,CTT,CTA,GCC,AAA,GTA,ATC,C 3′” | | |
| ex.23 | 53436-53415 | “Forward: 5′AAG,TGT,AAA,GTG,AGG,ACC,AAA,C 3′” | B | 391 bp |
| | 53047-53067 | “Reverse: 5′TTC,ATG,AGG,CTT,CAC,AGT,AGG 3′” | | |
| ex.24 | 50544-50523 | “Forward: 5′TCA,GCT,TTC,TAG,CAT,TGT,GAT,G 3′” | F | 354 bp |
| | 50191-50213 | “Reverse: 5′TTG,AAA,GGA,ATC,TAT,GAT,CTA,GG 3′” | | |
| ex.25 | 49262-49242 | “Forward: 5′TTC,TTC,TCA,TTG,CAG,AAC,ACA 3′” | F | 230 bp |
| | 49033-49052 | “Reverse: 5′TGT,GAA,AGT,GTG,CTC,ACC,AC 3′” | | |
| ex.26 | 43323-43302 | “Forward: 5′GAT,CTG,TGA,ACT,CTT,GTT,TTC,A 3′” | A | 244 bp |
| | 43080-43100 | “Reverse: 5′GAA,CAG,AGA,CTT,ACA,TTA,GGC 3′” | | |
| ex.27 | 39992-39971 | “Forward: 5′CAT,GAA,CCA,TTC,TTA,GCT,TCT,G 3′” | A | 451 bp |
| | 39543-39564 | “Reverse: 5′TTG,TGT,CAT,CTT,TAC,TCT,CCT, G 3′” | | |
| ex.28 | 38285-38263 | “Forward: 5′ATG,TGA,TTA,TGG,AAT,AGG,TTG,TC 3′” | A | 247 bp |
| | 38036-38058 | “Reverse: 5′TAT,TTA,ACA,TCT,CAT,ACA,GTC,AG 3′” | | |
| Promoter: | for promoter fragments: | | | |
| | (Accession: A0002457) | | | |
| fragment 1 | 138856-138876 | “Prom 1f (f): 5′CCA,AGG,ACT,GTT,GAA,AGT,AGC 3′” | A | 487 bp |
| | 139342-139322 | “Prom 3r (r): 5′TTG,CAT,ATG,CAA,GTG,TAC,AGC 3′” | | |
| fragment 2 | 139285-139304 | “Prom 2f (f): 5′CAC,AGG,GTT,GTT,GTT,AAC,CC 3′” | A | 574 bp |
| | 139858-139838 | “Prom 5r (r): 5′TCT,GAG,GAT,GTT,TCC,ACT,TTC 3′” | | |
| fragment 3 | 139809-139831 | “Prom 4f (f): 5′TTA,TGG,CTT,TGA,AGT,ATG,AGT,TA 3′” | B | 570 bp |
| | 140378-140358 | “Prom 6r (r): 5′GCA,TGC,TTG,ACA,GTT,TCT,GAG 3′” | | |
| fragment 4 | 140358-140378 | “Prom 6f (f): 5′CTC,AGA,AAC,TGT,CAA,GCA,TGC 3′” | A | 592 bp |
| | 140949-140931 | “Prom 7r (r): 5′TTG,GAA,CGG,CCA,CCA,AGA,C 3′” | | |
| Enhancer: | for enhancer fragments: | | | |
| | “(Accession: M57451, | | | |
| | J05674)” | | | |
| fragment 1 | pos. 1-19 | “Enh 1f (f): 5′CCC,TTC,TAA,CCA,TGG,CCA,G 3′” | A | 338 bp |
| | 338-320 | “Enh 3r (r): 5′GTG,CCT,CCT,GTC,AAT,GGT,G 3′” | | |
| fragment 2 | 279-297 | “Enh 2f (f): 5′CTA,CTG,AAA,CCG,CAG,CAT,G 3′” | A | 416 bp |
| | 694-676 | “Enh 4r (r): 5′TTG,CAG,ACA,CTG,ACT,CAC,C 3′” | | |

TABLE 2

| | | fre- | | frequency: | | |
|---|---|---|---|---|---|---|
| | | que- | fre- | homozygotes | | |
| | Position | ncy: | quency: | mutant | | |
| PCR | of the | heter- | homo-: | “(expected, | | |
| fragment | varia- | ozy | zygotes | Hardy- | | |
| name | tion | gotes | mutant | Weinberg)” | wt-sequence | wt/mut- and/or mut-sequence |
| | for exons | | | | | |
| | 1-7: | | | | | |
| | (Accession: | | | | | |
| | AC002457) | | | | | |
| ex.1 | 140837 | 4.17% | <4% | 0.04% | f: GCTCATTCGAGTAGCGGCTCT | wt/mut: |
| | | | | | r: AGAGCCGCTACTCGAATGAG | f: CTCATTCGAGT/CAGCGGCTCTT |
| | | | | | | r: AGAGCCGCTAIGCTCGAATGAG |
| | | | | | | mut: |
| | | | | | | f: CTCATTCGAGCAGGGGCTCTT |
| | | | | | | r: AGAGCCGCTGCTGGAATGAG |
| ex.2 | 141530 | 12.60% | <4% | 0.50% | f: CTTCAGGTCGGGATGGATCTTGA | wt/mut: |
| | | | | | r: CAAGATCCATCCCGACCTGA | f: CTTCAGGTCGGG/AATGGATCTTGA |
| | | | | | | r: CAAGATCCATC/TCCGACCTGA |
| | | | | | | mut: |
| | | | | | | f: CTTCAGGTCGGAATGGATCTTGA |
| | | | | | | r: CAAGATCCATTCCGACCTGA |
| ex.2 | 141590 | 15.50% | 0.97% | 1.20% | f: AAACTGAACAATAAAAGGTA | wt/mut: |
| | | | | | r: TACCTTTTATGTTCAGTTTAA | f: AAACTGAACA/GATAAAAGGTA |
| | | | | | | r: TACCTTTTATT/CGTTCAGTTTAA |
| | | | | | | mut: |
| | | | | | | f: AAACTGAACGATAAAAGGTA |
| | | | | | | r: TACCTTTTATCGTTCAGTTTAA |
| ex.5 | 171466 | 26.08% | 4.34% | 2.90% | f: GACATAAATGGTATGTTTGTTT | wt/mut: |
| | | | | | r: AACAAACATACCATTTATGTCT | f: AGACATAAATGG/TTATGTTTGT |
| | | | | | | r: AACAAACATAC/ACATTTATGTC |
| | | | | | | mut: |
| | | | | | | f:AGACATAAATGTTATGTTTGT |
| | | | | | | r: AACAAACATAACATTTATGTC |
| ex.5 | 171512 | <2% | <2% | <2% | f: GATACAGGGTTCTTGATGAAT | wt/mut: |
| | | | | | r: ATTCATGMGAACCCTGTATC | f: GATACAGGGTT/CCTTCATGAAT |
| | | | | | | r: ATTCATGAAGA/GACCCTGTATC |
| | | | | | | mut: |
| | | | | | | f: GATACAGGGTCCTTCATGAAT |
| | | | | | | r: ATTCATGAAGGACCCTGTATC |
| ex.6 | 175068 | 36.36% | 18.18% | 12.90% | f: TAAGCAGCAACAATGTCGTGTGC | wt/mut: |
| | | | | | | f: TAAGCAGCAAC/TAATGTCGTGTGC |
| | | | | | | mut: |
| | | | | | | f: AGCAGCAATAATGTCGTGT |
| | for exons | | | | | |
| | 11 + 12: | | | | | |
| | (Accession: | | | | | |
| | “M29432, | | | | | |
| | J05168)” | | | | | |
| ex.11 | 101 | 13.64% | <4% | 0.50% | f: TTCACTTCAGTTACCCATC | wt/mut: |
| | | | | | r: ATGGGTAACTGAAGTGAA | f: TCACTTCAG/ATTACCCATC |
| | | | | | | r: ATGGGTAAC/TTGAAGTGAA |
| | | | | | | mut: |
| | | | | | | f: TCACTTCAATTACCCATC |
| | | | | | | r: ATGGGTAATTGAAGTGAA |
| ex.12 | 308 | 52.2% | 12.50% | 15.20% | f: CTTGAAGGGCCTGAACCTGA | f: TCTTGAAGGGCTCTGAACCTG |
| | | | | | r: CAGGTTCAGGCCCTTCAAGA | r: TCAGGTTCAGG/ACCCTTCAAGA |
| | for exon 12: | | | | | mut/mut: |
| | (Accession: | | | | | f: CTTGAAGGGTCTGAACCT |
| | AC005068) | | | | | r: GGTTCAGACCCTTCAAG |
| ex.12 | 83946 | 8.70% | <4% | 0.16% | f: TCAGCAGTCACATTGCA | wt/mut: |
| | | | | | | f: CAGCAGTC/TACATTGCAC |
| | | | | | | mut: |
| | | | | | | f: CAGCAGTTACATTGCAC |
| | for exon 17: | | | | | |
| | (Accession: | | | | | |
| | AC005068) | | | | | |
| ex.17 | 78170 | 45.83% | 12.50% | 12.90% | f: CAAAAATTAGTAAAGGAATA | wt/mut: |
| | | | | | | r: ACAAAAATTA/TGTAAAGGAAT |
| | | | | | | mut/mut: |
| | | | | | | r: CAAAAATTTGTAAAGGAATA |
| | for exon 26: | | | | | |
| | (Accession: | | | | | |
| | “M29445, | | | | | |
| | J05168)” | | | | | |
| ex.26 | 176 | 54.16% | 25% | 27% | f: GAAGAGATCGTGAGGG | wt/mut: |
| | | | | | | f: GAAGAGATC/TGTGAGGGC |
| | | | | | | mut/mut: |
| | | | | | | f: AAGAGATTGTGAGGGCA |
| ex. 5 | 171456 | 8, 33% | <4% | 0, 20% | f: GACTAAAGAGACATAAATG wt/mut: | |
| | | | | | r: CATTTATGTCTCTTTAGTC | f: GACTAAAGAG/CACATAA |
| | | | | | | r: GATTrATGTG,GTCTTTAGTC |
| | | | | | | mut: |
| | | | | | | f: GACTAAAGACACATAAATG |
| | | | | | | r: CATTTATGTGTCTTTAGTC |
| ex.5 | 171404 | 4, 17% | <4% | 0, 04% | f: ATCATTAAATGAAATGAGT | wt/mut: |
| | | | | | r: AGTCATTTCATTTAATGAT | f: ATCATTAAAT/CGAAATGAGT |
| | | | | | | r: AGTCATTTCA/GTTTAATGAT |
| | | | | | | mut: |
| | | | | | | f: ATCATTAAACGAAATGAGT |
| | | | | | | r: ACTCATTTCGTTTAATGAT |
| ex.6 | 175074 | 8, 33% | <4% | 0, 20% | f: CAACAATGTCGTGTGCATC | wt/mut: |
| | | | | | r: GATGCACACGACATTGTTG | f: CAACAATGTC/TGTGTGCATC |
| | | | | | | r: GATGCACACG/AACATTGTTG |
| | | | | | | mut: |
| | | | | | | f: CAACAATGTTGTGTGCATC |
| | | | | | | r: GATGCACACAACATTGTTG |
| | for exon 17: | | | | | |
| | (Accession: | | | | | |
| | AC005068) | | | | | |
| ex.17 | 77811 | 4, 17% | <4% | 0, 04% | f: GGCTTGAAGATGTAAGAAT | wt/mut: |
| | | | | | r: ATTCTTACATCTTCAAGCC | f: GGCTTGAAGA/GTGTAAGAAT |
| | | | | | | r: ATTCTTACAT/CCTTCAAGCC |
| | | | | | | mut: |
| | | | | | | f: GGCTTGAAGGTGTAAGAAT |
| | | | | | | r: ATTCTTACACCTTCAAGCC |
| | for exon 26: | | | | | |
| | (Accession: | | | | | |
| | M29445, | | | | | |
| | J05168) | | | | | |
| ex.26 | 137 | 4, 17% | <4% | 0, 04% | f: GAACATTGCCTATGGAGAC | wt/mut |
| | | | | | r: GTCTCCATAGGCAATGTTC | f: GAACATTGCC/TTATGGAGAC |
| | | | | | | r: GTCTCCATAG/AGCAATGTTC |
| | | | | | | mut: |
| | | | | | | f: GAACATTGCTTATGGAGAC |
| | | | | | | r: GTCTCCATAAGCAATGTTC |

TABLE 3

| PCR | | | | | |
|---|---|---|---|---|---|
| fragment | | | | frag- | PCR |
| with a | Position of | specific wildtype primer/ | primer | ment | condi- |
| variation | the variation | specific mutant primer | common primer | size | tions |
| | for exons 1-7: | | | | |
| | (Accession: | | | | |
| | AC002457) | | | | |
| ex.2 | 141530 | “Pos.−1Gf | “Exon 2r: 5′ CTC,AGC,CAA,CM,ACT.TCT,GC 3′ ” | 251 bp | |
| | | (wt): | | | |
| | | 5′ GGT,TTC,TCT, | | | |
| | | TCA,GGT,CGG, | | | |
| | | G 3′” | | | |
| | | “Pos.−1Af | | | |
| | | (mut): | | | |
| | | 5′ CGG,TTT,CTC, | | | |
| | | TTC,AGG,TCG, | | | |
| | | GA 3′” | | | |
| ex.2 | 111590 | “Pos.61Ar | “Exon 2f: 5′ TGT,TAC,TGG,TCT,CTG,GGC,TTC 3′ ” | 190 bp | |
| | | (wt): | | | |
| | | 5′ GAA,ACA,AGC, | | | |
| | | TAG,TTA,CCT,TTT, | | | |
| | | ATT 3′” | | | |
| | | “Pos.61Gr | | | |
| | | (mut): | | | |
| | | 5′ AAA,CAA,GCT, | | | |
| | | AGT,TAC,CTT,TTA, | | | |
| | | TC 3′” | | | |
| ex.5 | 171466 | “Pos.−25Gr | “Exon 5f: 5′ CAC,ACA,GTC,AGC,AGA,GAA,GT 3′ ” | 180 bp | |
| | | (wt): | | | |
| | | 5′ GAC,CAC,CAC, | | | |
| | | AAA,ACA,AAC,ATA, | | | |
| | | C 3′” | | | |
| | | “Pos.−25Tr | | | |
| | | (mut): | | | |
| | | 5′ GAC,CAC,CAC, | | | |
| | | AAA,ACA,AAC,ATA, | | | |
| | | A 3′” | | | |
| ex.5 | 171512 | “Pos.308Tr | “Exon 5f: 5′ CAC,ACA,GTC,AGC,AGA,GAA,GT 3′ ” | 225 bp | |
| | | (wt): | | | |
| | | 5′ CTT,CCT,CCA, | | | |
| | | GAT,TCA,TGA, | | | |
| | | AGA 3′” | | | |
| | | “Pos.308Cr | | | |
| | | (mut): | | | |
| | | 5′ CTT,CCT,CCA, | | | |
| | | GAT,TCA,TGA, | | | |
| | | AGG 3′” | | | |
| ex.6 | 175058 | “Pos.+139Cr | “Exon 6f: 5′ GGA,ATG,AGT,GGT,GTC,TTT,GG 3′ ” | 428 bp | |
| | | (wt): | | | |
| | | 5′ AAA,AGG,ATG, | | | |
| | | GAC,ACG,ACA, | | | |
| | | TTG 3′” | | | |
| | | “Pos.+139Tr | | | |
| | | (mut): | | | |
| | | 5′ CAA,AAG,GAT, | | | |
| | | GCA,CAC,GAC,ATT, | | | |
| | | A 3′” | | | |
| | for exons 11 + 12: | | | | |
| | (Accession: | | | | |
| | “M29432, J05168)” | | | | |
| ex.11 | 101 | “Pos.1199Gf | “Exon 11r:5′ACT,AGG,TTT,AAA,TAT,ACA,TGC,AC 3′ ” | 151 bp | |
| | | (wt): | | | |
| | | 5′ GAA,TTG,AGA, | | | |
| | | AAT,GTT,CAC,TTC, | | | |
| | | AG 3′” | | | |
| | | “Pos.1199Af | | | |
| | | (mut): | | | |
| | | 5′ GAA,TTC,AGA, | | | |
| | | AAT,GTT,CAC,TTC, | | | |
| | | AA 3′” | | | |
| ex.12 | 308 | “Pos.1236Cf | “Exon 12r: 5′ GGG,CAA,CAT,CAG,AAA,GAT,GTG 3′ ” | 205 bp | |
| | | (wt): | | | |
| | | 5′ CCT,GGT,AGA, | | | |
| | | TCT,TGA,AGG, | | | |
| | | GC 3′” | | | |
| | | “Pos.1236Tf | | | |
| | | (mut): | | | |
| | | 5′ CCT,GGT,AGA, | | | |
| | | TCT,TGA,AGG, | | | |
| | | GT 3′” | | | |
| | for exon 17: | | | | |
| | (Accession: | | | | |
| | AC005068) | | | | |
| ex.17 | 78170 | “Pos.−76Tf | “Exon 17r: 5′ TGG,GCA,TCA,CAC,TTA,CCC,C 3′ ” | 261 bp | |
| | | (wt): | | | |
| | | 5′ AGG,ATA,GGA, | | | |
| | | TAT,ATT,CCT,TTA, | | | |
| | | CT 3′” | | | |
| | | “Pos.−76Af | | | |
| | | (mut): | | | |
| | | 5′ AGG,ATA,GGA, | | | |
| | | TAT,ATT,CCT,TTA, | | | |
| | | CA 3′” | | | |
| | for exon 26: | | | | |
| | (Accession: | | | | |
| | “M29445, J05168)” | | | | |
| ex.26 | 176 | “Pos.3435Cr | “Exon 26f: 5′ GAT,CTG,TGA,ACT,CTT,GTT,TTC,A 3′ ” | 195 bp | |
| | | (wt): | | | |
| | | 5′ CTC,CTT,TGC, | | | |
| | | TGC,CCT,CAC, | | | |
| | | G 3′” | | | |
| | | “Pos.3435Tr | | | |
| | | (mut): | | | |
| | | 5′ CTC,CTT,TGC, | | | |
| | | TGC,CCT,CAC, | | | |
| | | A 3′” | | | |

TABLE 4

| | PGP | |
|---|---|---|
| | concen- | |
| samples | tration | MDR-1 genotype |
| not induced probands | 55 | T-allele present (T/T and T/C) |
| | 39 | at position 176 in |
| | 276 | Acc.#M29445/J05168 |
| | 376 | in exon 26 |
| not induced probands, mean | 212 | |
| rifampicine-induced probands | 142 | |
| | 1085 | |
| | 520 | |
| | 601 | |
| rifampicine-induced probands, | 587 | |
| mean | | |
| not induced probands | 96 | T-allele absent (C/C only) |
| | 302 | at position 176 in |
| | 291 | Acc.#M29445/J05168 |
| not induced probands, mean | 230 | |
| rifampicine-induced probands | 423 | |
| | 1264 | |
| | 1086 | |
| rifampicine-induced probands, | 924 | |
| mean | | |
| lowest rif-induced activity | 142.1 | homozygous T/T |
| | | at position 176 in |
| | | Acc.#M29445/J05168 |
| highest rif-induced activity | 1264.9 | homozygous C/C |
| | | at position 176 in |
| | | Acc.#M29445/J05168 |

TABLE 5

| | digoxin | |
|---|---|---|
| | concentration | |
| samples | in blood | MDR-1 genotype |
| not induced probands | 63.6 | T-allele present |
| | 64.1 | (T/T and T/C) |
| | 73.2 | at position 176 in |
| | 54.7 | Acc.#M29445/ |
| not induced probands, mean | 63.9 | J05168 in exon 26 |
| rifampicine-induced probands | 57.3 | |
| | 39 | |
| | 45.8 | |
| | 37.7 | |
| rifampicine-induced probands, | 45 | |
| mean | | |
| not induced probands | 55.6 | T-allele absent |
| | 30.8 | (C/C only) |
| | 48.3 | at position 176 in |
| not induced probands, mean | 44.9 | Acc.#M29445/ |
| rifampicine-induced probands | 39.6 | J05168 |
| | 12.3 | |
| | 33.9 | |
| rifampicine-induced probands, | 28.6 | |
| mean | | |
| lowest rif-induced dig | 57.3 | homozygous T/T |
| blood level | | at position 176 in |
| | | Acc.#M29445/ |
| | | J05168 |
| highest rif-induced dig | 12.3 | homozygous C/C |
| blood level | | at position 176 in |
| | | Acc.#M29445/ |
| | | J05168 |

TABLE 8
NEW MDR-1 SNP′s

| | | | | - | | |
|---|---|---|---|---|---|---|
| | | | | frequency: | | |
| | | | | homozygotes | | |
| | | frequen- | | mutant | | |
| PCR | Position | cy: | frequency: | “(expected, | | |
| fragment | of the | heterozy | homozygotes | Hardy- | | wt/mut- and/or |
| name | variation | gotes | mutant | Weinberg)” | wt-sequence | mut-sequence |
| | for promoter | | | | | |
| | fragments | | | | | |
| | 1-4 and for | | | | | |
| | exons 3-6: | | | | | |
| | (Accession: | | | | | |
| | AC002457) | | | | | |
| promoter | 139015 | 3% * | <1% * | 0.02%* | f: AACTTAGTTATATCTTTGA | wt/mut: |
| fragment | | *: fre- | | | r: TCAAAGATATAAGTAAGTT | f:AACTTACTTA/GTATCTTTGA |
| 1 | | quencies | | | | r:TCAAAGATAT/CAAGTAAGTT |
| | | in | | | | mut: |
| | | Africans- | | | | f: AACTTACTTGTATCTTTGA |
| | | Americans | | | | r: TCAAAGATACAAGTAAGTT |
| promoter | 139064 | 1.5% | <1% | 0.01% | f: AGAAATAGTATAATCAACA | wt/mut: |
| fragment | | | | | r: TGTTGATTATACTATTTCT | f: AGAAATAGTA/TTAATCAACA |
| 1 | | | | | | r: TGTTGATTAT/AACTATTTCT |
| | | | | | | mut: |
| | | | | | | f: AGAAATAGTTTAATCAACA |
| | | | | | | r: TGTTGATTAAACTATTTCT |
| promoter | 139119 | 24.2% | 3% * | 2.3% * | f: TAGGGAGGGTTTAAGGCCA | wt/mut: |
| fragment | | *: fre- | | | r: TGGCCTTAAACCCTCCCTA | f: TAGGGAGGGT/CTTAAGGCCA |
| 1 | | quencies | | | | r: TGGCCTTAAA/GCCCTCCCTA |
| | | in | | | | mut: |
| | | Africans- | | | | t: TAGGGAGGGCTTAAGGCCA |
| | | Americans | | | | r: TGGCCTTAAGCCCTCCCTA |
| promoter | 139177 | 1.5% | 1.5% | 0.05% | f: GAAAGGTGAGATAAAGCAA | wt/mut: |
| fragment | | | | | r: TTGCTTTATCTCACCTTTC | f: GAAAGGTGAG/AATAAAGCAA |
| 1 | | | | | | r: TTGCTTTATC/TTCACCTTTC |
| | | | | | | mut: |
| | | | | | | f: GAAAGGTGAAATAAAGCAA |
| | | | | | | r: TTGCTTTATTTCACCTTTC |
| promoter | 139276 | 6.7% | <1% * | 0.1%* | f: CATTTACCCCAGATGGACC | wt/mut: |
| fragment | | *: fre- | | | r: GGTCCATCTGGGGTAAATG | f: CATTTACCCC/TAGATGGACC |
| 1 | | quencies | | | | r: GGTCCATCTG/AGGGTAAATG |
| | | in | | | | mut: |
| | | Africans- | | | | f: CATTTACCCTAGATGGACC |
| | | Americans | | | | r: GGTCCATCTAGGGTAAATG |
| promoter | 139479 | 9.7% | <1% | 0.2% | f: GAGGCGGGCGGARCACGAG | wt/mut: |
| fragment | | | | | r: CTCGTGATCCGCCCGCCTC | f: GAGGCGGGCG/AGATCACGAG |
| 2 | | | | | | r: CTCGTGATCC/TGCCCGCCTC |
| | | | | | | mut: |
| | | | | | | f: GAGGCGGGCAGATCACGAG |
| | | | | | | r: CTCGTGATCTGCCCGCCTC |
| promoter | 139619 | 12.1% | <1 | 0.4 | f: GGAGAATGGTGTGAACCCG | wt/mut: |
| fragment | | | | | r: CGGGTTCACACCATTCTCC | f: GGAGAATGGT/CGTGAACCCG |
| 2 | | | | | | r: CGGGTTCACA/GCCATTCTCC |
| | | | | | | mut: |
| | | | | | | f: GGAGAATGGCGTGAACCCG |
| | | | | | | r: CGGGTTCACGCCATTCTCC |
| promoter | 140118 | 1.5% | <1% | 0.01% | f: ATATGGAAGGAAATTACAA | wt/mut: |
| fragment | | | | | r: TTGTAATTTCCTTCCATAT | f: ATATGGAAGG/AAAATTACAA |
| 3 | | | | | | r: TTGTAATTTC/TCTTCCATAT |
| | | | | | | mut: |
| | | | | | | f: ATATGGAAGAAAATTACAA |
| | | | | | | r: TTGTAATTTTCTTCCATAT |
| promoter | 140216 | 3.1% * | <1% * | 0.03% * | f: AACACGGGCATTGATCTGA | wt/mut: |
| fragment | | *: fre- | | | r: TCAGATCAATGCCCGTGTT | f: AACACGGGCA/GTTGATCTGA |
| 3 | | quencies | | | | r: TCAGATCAAT/CGCCCGTGTT |
| | | in | | | | mut: |
| | | Africans- | | | | f: AACACGGGCGTTGATCTGA |
| | | Americans | | | | r: TCAGATCAACGCCCGTGTT |
| promoter | 140490 | 5.9% | <1% * | 0.08% * | f: TGTATTAAATGCGAATCCC | wt/mut: |
| fragment | | *: fre- | | | r: GGGATTCGCATTTAATACA | f: TGTATTAAAT/CGCGAATCCC |
| 4 | | quencies | | | | r: GGGATTCGCA/GTTTAATACA |
| | | in | | | | mut: |
| | | Africans- | | | | f: TGTATTAAACGCGAATCCC |
| | | Americans | | | | r: GGGATTCGCGTTTAATACA |
| promoter | 140568 | 2.9% | <1% * | 0.02% * | f: TTGAAAGACGTGTCTACAT | wt/mut: |
| fragment | | *: fre- | | | r: ATGTAGACACGTCTTTCAA | f: TTGAAAGACG/ATGTCTACAT |
| 4 | | quencies | | | | r: ATGTAGACAC/TGTCTTTCAA |
| | | in | | | | mut: |
| | | Africans- | | | | f: TTGAAAGACATGTGTCTACAT |
| | | Americans | | | | r: ATGTAGACATGTCTTTCAA |
| promoter | 140576 | 10.2% * | <1% * | 0.3% * | f: CGTGTCTACATAAGTTGAA | wt/mut: |
| fragment | | *: fre- | | | r: TTCAACTTATGTAGACACG | f: CGTGTCTACA/TTAAGTTGAA |
| 4 | | quencies | | | | r: TTCAACTTAT/AGTAGACACG |
| | | in | | | | mut: |
| | | Africans- | | | | f: CGTGTCTACTTAAGTTGAA |
| | | Americans | | | | r: TTCAACTTAAGTAGACACG |
| promoter | 140595 | 5% * | <1% * | 0.06% * | f: ATGTCCCCAATGATTCAGC | wt/mut: |
| fragment | | *: fre- | | | r: GCTGAATCATTGGGGACAT | f: ATGTCCCCAA/GTGATTCAGC |
| 4 | | quencies | | | | r: GCTGAATCAT/CTGGGGACAT |
| | | in | | | | mut: |
| | | Africans- | | | | f: ATGTCCCCAGTGATTCAGC |
| | | Americans | | | | r: GCTGAATCACTGGGGACAT |
| promoter | 140727 | 3.1% | <1% * | 0.03% * | f: CCGGGCCGGGAGCAGTCAT | wt/mut: |
| fragment | | *: fre- | | | r: ATGACTGCTCCCGGCCCGG | f: CCGGGCCGGG/AAGCAGTCAT |
| 4 | | quencies | | | | r: ATGACTGCTC/TCCGGCCCGG |
| | | in | | | | mut: |
| | | Africans- | | | | f: CCGGGCCGGAAGCAGTCAT |
| | | Americans | | | | r: ATGACTGCTTCCGGCCCGG |
| ex. 3 | 145984 | 8.3% | <1% | 0.2% | f: AAAATACTTCGGAAATTTG | wt/mut: |
| | | | | | r: CAAATTTCCGAAGTATTTT | f: AAAATACTTC/TGGAAATTTG |
| | | | | | | r: CAAATTTCCG/AAAGTATTTT |
| | | | | | | mut: |
| | | | | | | f: AAAATACTTTGGAAATTTG |
| | | | | | | r: CAAATTTCCAAAGTATTTT |
| ex. 5 | 171511 | 1.5% | <1% | 0.01% | f: GATACAGGGTTCTTCATGA | wt/mut: |
| | | | | | r: TCATGAAGAACCCTGTATC | f: GATACAGGGT/CTCTTCATGA |
| | | | | | | r: TCATGAAGAA/GCCCTGTATC |
| | | | | | | mut: |
| | | | | | | f: GATACAGGGCTCTTCATGA |
| | | | | | | r: TCATGAAGAGCCCTGTATC |
| ex. 6 | 174901 | 1.1% | <1% | 0.003% | f: GTGCACGATGTTGGGGAGC | wt/mut: |
| | | | | | r: GCTCCCCAACATCGTGCAC | f: GTGCACGATG/ATTGGGGAGC |
| | | | | | | r: GCTCCCCAAC/TATCGTGCAC |
| | | | | | | mut: |
| | | | | | | f: GTGCACGATATTGGGGAGC |
| | | | | | | r: GCTCCCCAATATCGTGCAC |
| ex. 6 | 175142 | 16.3% | <1% | 0.7% | f: CATTAAATGAAGGACTGGG | wt/mut: |
| | | | | | r: CCCAGTCCTTCATTTAATG | f: CATTAAATGA/GAGGACTGGG |
| | | | | | | r: CCCAGTCCTT/CCATTTAATG |
| | | | | | | mut: |
| | | | | | | f: CATTAAATGGAGGACTGGG |
| | | | | | | r: CCCAGTCCTCCATTTAATG |
| ex. 6 | 175181 | 46.9% | 20.3% | 19.4% | f: TCCTCTGAGAATGTGCAGT | wt/mut: |
| | | | | | r: ACTGCACATTCTCAGAGGA | f: TCCTCTGAGA/GATGTGCAGT |
| | | | | | | r: ACTGCACATT/CCTCAGAGGA |
| | | | | | | mut: |
| | | | | | | f: TCCTCTGAGGATGTGCAGT |
| | | | | | | r: ACTGCACATCCTCAGAGGA |
| | for exons | | | | | |
| | 10-26: | | | | | |
| | (Accession: | | | | | |
| | AC005068) | | | | | |
| ex. 10 | 84701 | 45.8% | 25% | 23% | f: AAAATTGCTGTCACTATCT | wt/mut: |
| | | | | | r: AGATAGTGACAGCAATTTT | f: AAAATTGCTG/ATCACTATCT |
| | | | | | | r: AGATAGTGAC/TAGCAATTTT |
| | | | | | | mut: |
| | | | | | | f: AAAATTGCTATCACTATCT |
| | | | | | | r: AGATAGTGATAGCAATTTT |
| ex. 12 | 84032 | 0.5% | <1% | 0.001% | f: GAGCACAACAGTCCAGCTG | wt/mut: |
| | | | | | r: CAGCTGGACTGTTGTGCTC | f: GAGCACAACA/GGTCCAGCTG |
| | | | | | | r: CAGCTGGACT/CGTTGTGCTC |
| | | | | | | mut: |
| | | | | | | f: GAGCACAACGGTCCAGCTG |
| | | | | | | r: CAGCTGGACCGTTGTGCTC |
| ex. 12 | 84074 | 3.4% | <1% | 0.03% | f: TGGGCAGACGGTGGCCCTG | wt/mut: |
| | | *: fre- | | | r: CAGGGCCACCGTCTGCCCA | f: TGGGCAGACG/AGTGGCCCTG |
| | | quencies | | | | r: CAGGGCCACC/TGTCTGCCCA |
| | | in | | | | mut: |
| | | Africans- | | | | f: TGGGAGACAGTGGCCCTG |
| | | Americans | | | | r: CAGGGCCACTGTCTCCCA |
| ex. 12 | 84119 | 3.4% | <1% | 0.03% | f: CTCGTCCTGGTAGATCTTG | wt/mut: |
| | | *: fre- | | | r: CAAGATCTACCAGGACGAG | f: CTCGTCCTGG/ATAGATCTTG |
| | | quencies | | | | r: CAAGATCTAC/TCAGGACGAG |
| | | in | | | | mut: |
| | | Africans- | | | | f: CTCGTCCTGATAGATCTTG |
| | | Americans | | | | r: CAAGATCTATCAGGACGAG |
| ex. 12 | 83973 | 3.4% | <1% | 0.03% | f: GACCCATGCGAGCTAGACC | wt/mut: |
| | | *: fre- | | | r: GGTCTAGCTCGCATGGGTC | f: GACCCATGCG/AAGCTAGACC |
| | | quencies | | | | r: GGTCTAGCTC/TGCATGGGTC |
| | | in | | | | mut: |
| | | Africans- | | | | f: GACCCATGCAAGCTAGACC |
| | | Americans | | | | r: GGTCTAGCTTGCATGGGTC |
| ex. 19 | 73252 | 8.3% | <1% | 0.2% | f: ACTTTGTCTAATCTCCTGC | wt/mut: |
| | | | | | r: GCAGGAGATTAGACAAAGT | f: ACTTTGTCTA/GATCTCCTGC |
| | | | | | | r: GCAGGAGATT/CAGACAAAGT |
| | | | | | | mut: |
| | | | | | | f: ACTTTGTCTGATCTCCTGC |
| | | | | | | r: GCAGGAGATCAGACAAAGT |
| ex. 20 | 70371 | 3.8% | <1% | 0.04% | f: AATCATTTTCTGTGCCACA | wt/mut: |
| | | *: fre- | | | r: TGTGGCACAGAAAATGATT | f: AATCATTTTC/ATGTGCCACA |
| | | quencies | | | | r: TGTGGCACAG/TAAAATGATT |
| | | in | | | | mut: |
| | | Africans- | | | | f: AATCATTTTATGTGCCACA |
| | | Americans | | | | r: TGTGGCACATAAAATGATT |
| ex. 20 | 70253 | 22.2% | <1% | 1.2% | f: TCTACTGGTGTTTGTCTTA | wt/mut: |
| | | | | | r: TAAGACAAACACCAGTAGA | f: TCTACTGGTG/ATTTGTCTTA |
| | | | | | | r: TAAGACAAAC/TACCAGTAGA |
| | | | | | | mut: |
| | | | | | | f: TCTACTGGTATTTGTCTTA |
| | | | | | | r: TAAGACAAATACCAGTAGA |
| ex. 20 | 70237 | 16.7% | <1% | 0.6% | f: TTAATTGGCCATTTTGGAC | wt/mut: |
| | | | | | r: GTCCAAAATGGCCAATTAA | f: TTAATTGGCC/TATTTTGGAC |
| | | | | | | r: GTCCAAAATG/AGCCAATTAA |
| | | | | | | mut: |
| | | | | | | f: TTAATTGGCTATTTTGGAC |
| | | | | | | r: GTCCAAAATAGCCAATTAA |
| ex. 20 | 70204 | 16.7% | <1% | 0.6% | f: AATTTTCTCCTTACGGGTG | wt/mut: |
| | | | | | r: CACCCGTAAGGAGAAAATT | f: AATTTTCTCC/ATTACGGGTG |
| | | | | | | r: CACCCGTAAG/TGAGAAAATT |
| | | | | | | mut: |
| | | | | | | f: AATTTTCTCATTACGGGTG |
| | | | | | | r: CACCCGTAATGAGAAAATT |
| ex. 20 | 70200 | 4.2% | <1% | 0.04% | f: TTCTCCTTACGGGTGTTAG | wt/mut: |
| | | | | | r: CTAACACCCGTAAGGAGAA | f: TTCTCCTTAC/TGGTGTTAG |
| | | | | | | r: CTAACACCG/ATAAGGAGAA |
| | | | | | | mut: |
| | | | | | | f: TTCTCCTTATGGGTGTTAG |
| | | | | | | r: CTAACACCCATAAGGAGAA |
| ex. 26 | 43263 | 0.5% | <1% | 0.001% | f: TGAATGTTCAGTGGCTCCG | wt/mut: |
| | | | | | r: CGGAGCCACTGAACATTCA | f: TGAATGTTCA/CGTGGCTCCG |
| | | | | | | r: CGGAGCCACT/GGAACATTCA |
| | | | | | | mut: |
| | | | | | | f: TGAATGTTCCGTGGCTCCG |
| | | | | | | r: CGGAGCCACGGAACATTCA |
| ex. 26 | 43162 | 0.5% | <1% | 0.001% | f: CGGGTGGTGTCACAGGAAG | wt/mut: |
| | | | | | r: CTTCCTGTGACACCACCCG | f: CGGGTGGTGT/ACACAGGAAG |
| | | | | | | r: CTTCCTGTGA/TCACCACCCG |
| | | | | | | mut: |
| | | | | | | f: CGGGTGGTGACACAGGAAG |
| | | | | | | r: CTTCCTGTGTCACCACCCG |

Legend for table 8: New MDR-1 SNP's
MDR1 variations and the respective widetype and mutant alleles: The expected frequency for homozygotes for the mutant allele were calculated on the basis of the Hardy-Weinberg distribution. In the sequence section the deviant bases are underlined and in bold style.

1-45. (canceled)

46. A method of diagnosing acquired multidrug resistance or sensitivity related to the presence of a molecular variant of the MDR-1 gene comprising determining in a sample from a subject the presence of a polynucleotide selected from the group consisting of:
(a) a polynucleotide having the nucleic acid sequence of SEQ ID NO 122;
(b) a polynucleotide encoding a molecular variant Multi Drug Resistance (MDR)-1 polypeptide, wherein said polynucleotide is having at a position corresponding to position 176 of the MDR-1 gene (Accession No: M29445 or J05168) a nucleotide exchange;
(c) a polynucleotide encoding a molecular variant MDR-1 polypeptide, wherein said polynucleotide is having at a position corresponding to position 176 of the MDR-1 gene (Accession No: M29445 or J05168) a T; and
(d) a nucleic acid molecule complementary to the polynucleotide of (a) to (c);
wherein the presence of said polynucleotide is considered indicative of said acquired multidrug resistance or sensitivity in said method.

47. The method of claim 46, wherein the nucleotide substitution results in altered expression of the variant MDR-1 gene compared to the corresponding wild type gene.

48. The method of claim 46, wherein said acquired multidrug resistance is in tumors and other diseases, the therapy of which is dependent on drug treatment.

49. The method of claim 47, wherein said acquired multidrug resistance is in tumors and other diseases, the therapy of which is dependent on drug treatment.

50. A method of diagnosing a disorder related to the presence of a molecular variant of the MDR-1 gene or susceptibility to such a disorder, comprising determining in a sample from a subject the presence of a polynucleotide selected from the group consisting of:
(a) a polynucleotide having the nucleic acid sequence of SEQ ID NO 122;
(b) a polynucleotide encoding a molecular variant Multi Drug Resistance (MDR)-1 polypeptide, wherein said polynucleotide is having at a position corresponding to position 176 of the MDR-1 gene (Accession No: M29445 or J05168) a nucleotide exchange;
(c) a polynucleotide encoding a molecular variant MDR-1 polypeptide, wherein said polynucleotide is having at a position corresponding to position 176 of the MDR-1 gene (Accession No: M29445 or J05168) a T; and
(d) a nucleic acid molecule complementary to the polynucleotide of (a) to (c);
wherein the presence of said polynucleotide is considered indicative of said acquired multidrug resistance or sensitivity in said method.

51. The method of claim 50, wherein said disorder is cancer.

52. The method of any one of claims 46 to 51, comprising PCR, ligase chain reaction, restriction digestion, direct sequencing, nucleic acid amplification techniques, hybridization techniques or immunoassays.

53. The method of any one of claims 46 to 51, wherein an oligo- or polynucleotide is used for diagnosing acquired multidrug resistance or sensitivity related to the presence of a molecular variant of the MDR-1 gene.

54. The method of claim 52, wherein an oligo- or polynucleotide is used for diagnosing acquired multidrug resistance or sensitivity related to the presence of a molecular variant of the MDR-1 gene.

55. The method of claim 53, wherein said oligonucleotide is about 15 to 50 nucleotides in length and comprises the nucleotide sequence of SEQ ID NOs 122 or a complementary sequence of any one of those.

56. The method of claim 54, wherein said oligonucleotide is about 15 to 50 nucleotides in length and comprises the nucleotide sequence of SEQ ID NOs 122 or a complementary sequence of any one of those.

57. A method for the prediction of blood levels of a MDR-1 substrate and/or inducer for improvement of drug safety and efficacy, to predict and prevent side effects and drug interactions and/or to increase patient compliance comprising using a MDR-1 gene single nucleotide polymorphism (SNP) MDR-1 in exon 26 (C3435T) as a pharmacogenetic factor.

58. The method of claim 57, wherein the substrate and/or inducer are selected from anticonvulsant/antiepileptic drugs, cardiac glycosides, immunosuppressive drugs, macrolid-antibiotics, or macrocyclic-antibiotics.

59. A diagnostic composition comprising a polynucleotide selected from the group consisting of:
(a) a polynucleotide having the nucleic acid sequence of SEQ ID NO 122;
(b) a polynucleotide encoding a molecular variant Multi Drug Resistance (MDR)-1 polypeptide, wherein said polynucleotide is having at a position corresponding to position 176 of the MDR-1 gene (Accession No: M29445 or J05168) a nucleotide exchange;
(c) a polynucleotide encoding a molecular variant MDR-1 polypeptide, wherein said polynucleotide is having at a position corresponding to position 176 of the MDR-1 gene (Accession No: M29445 or J05168) a T; and
(d) a nucleic acid molecule complementary to the polynucleotide of (a) to (c).

60. A diagnostic composition comprising the primer or probe as defined in claim 53.

61. A diagnostic composition comprising the primer or probe as defined in claim 54.


SEQUENCE LISTING (376 sequences)

SEQ ID NO: 1 - DNA, 20 bp, Artificial Sequence
  Description of Artificial Sequence synthetic
cccttaactacgtcctgtag

SEQ ID NO: 2 - DNA, 20 bp, Artificial Sequence
  Description of Artificial Sequence synthetic
gaggacttcacactatccac

SEQ ID NO: 3 - DNA, 21 bp, Artificial Sequence
  Description of Artificial Sequence synthetic
tcttactgctctctgggcttc

SEQ ID NO: 4 - DNA, 20 bp, Artificial Sequence
  Description of Artificial Sequence synthetic
ctcagccaacaaacttctgc

SEQ ID NO: 5 - DNA, 20 bp, Artificial Sequence
  Description of Artificial Sequence synthetic
cactcagtgataaccacgta

SEQ ID NO: 6 - DNA, 20 bp, Artificial Sequence
  Description of Artificial Sequence synthetic
gcatctccattaacataccc

SEQ ID NO: 7 - DNA, 20 bp, Artificial Sequence
  Description of Artificial Sequence synthetic
gggtgtcttggactaggttg

SEQ ID NO: 8 - DNA, 20 bp, Artificial Sequence
  Description of Artificial Sequence synthetic
tgcctcctacaggactaaac

SEQ ID NO: 9 - DNA, 20 bp, Artificial Sequence
  Description of Artificial Sequence synthetic
cacacagtcagcagagaagt

SEQ ID NO: 10 - DNA, 22 bp, Artificial Sequence
  Description of Artificial Sequence synthetic
actatcaagagtattgttctcc

SEQ ID NO: 11 - DNA, 20 bp, Artificial Sequence
  Description of Artificial Sequence synthetic
ggaatgagtggtctctttgg

SEQ ID NO: 12 - DNA, 20 bp, Artificial Sequence
  Description of Artificial Sequence synthetic
aaggcactgggaacaaaagg

SEQ ID NO: 13 - DNA, 21 bp, Artificial Sequence
  Description of Artificial Sequence synthetic
tcctagtagaaacttctaccc

SEQ ID NO: 14 - DNA, 19 bp, Artificial Sequence
  Description of Artificial Sequence synthetic
ttccgtagggtgagagcag

SEQ ID NO: 15 - DNA, 21 bp, Artificial Sequence
  Description of Artificial Sequence synthetic
cagattttgctctacacatgc

SEQ ID NO: 16 - DNA, 23 bp, Artificial Sequence
  Description of Artificial Sequence synthetic
attagttatgctgtaatacatcc

SEQ ID NO: 17 - DNA, 22 bp, Artificial Sequence
  Description of Artificial Sequence synthetic
catgtatatcacaggactgaac

SEQ ID NO: 18 - DNA, 22 bp, Artificial Sequence
  Description of Artificial Sequence synthetic
ctagtagtgcatatgtctgtag

SEQ ID NO: 19 - DNA, 21 bp, Artificial Sequence
  Description of Artificial Sequence synthetic
gtgacagaatgagaacctgtc

SEQ ID NO: 20 - DNA, 21 bp, Artificial Sequence
  Description of Artificial Sequence synthetic
tggagagctggataaagtgac

SEQ ID NO: 21 - DNA, 23 bp, Artificial Sequence
  Description of Artificial Sequence synthetic
aaattgatctgttagaagccaag

SEQ ID NO: 22 - DNA, 23 bp, Artificial Sequence
  Description of Artificial Sequence synthetic
actaggtttaaatatacatgcac

SEQ ID NO: 23 - DNA, 21 bp, Artificial Sequence
  Description of Artificial Sequence synthetic
gaacagtcagttcctatatcc

SEQ ID NO: 24 - DNA, 21 bp, Artificial Sequence
  Description of Artificial Sequence synthetic
gggcaacatcagaaagatgtg

SEQ ID NO: 25 - DNA, 21 bp, Artificial Sequence
  Description of Artificial Sequence synthetic
cacatctttctgatgttgccc

SEQ ID NO: 26 - DNA, 19 bp, Artificial Sequence
  Description of Artificial Sequence synthetic
aagggcaaagggcaaggac

SEQ ID NO: 27 - DNA, 21 bp, Artificial Sequence
  Description of Artificial Sequence synthetic
tgggttttctgtggtagaaat

SEQ ID NO: 28 - DNA, 20 bp, Artificial Sequence
  Description of Artificial Sequence synthetic
gttggtttgaactaagcctc

SEQ ID NO: 29 - DNA, 21 bp, Artificial Sequence
  Description of Artificial Sequence synthetic
aaatttctctctctttaggcc

SEQ ID NO: 30 - DNA, 22 bp, Artificial Sequence
  Description of Artificial Sequence synthetic
ttctgaagttaaactatacctg

SEQ ID NO: 31 - DNA, 23 bp, Artificial Sequence
  Description of Artificial Sequence synthetic
ttcttatttattttagacagcag

SEQ ID NO: 32 - DNA, 19 bp, Artificial Sequence
  Description of Artificial Sequence synthetic
gcatctcccttcataccag

SEQ ID NO: 33 - DNA, 23 bp, Artificial Sequence
  Description of Artificial Sequence synthetic
cattgataaggaataaggatagg

SEQ ID NO: 34 - DNA, 20 bp, Artificial Sequence
  Description of Artificial Sequence synthetic
cctatcacaaaccagactgc

SEQ ID NO: 35 - DNA, 21 bp, Artificial Sequence
  Description of Artificial Sequence synthetic
atttccagcgtactaaggctc

SEQ ID NO: 36 - DNA, 21 bp, Artificial Sequence
  Description of Artificial Sequence synthetic
acttggctgttagtagccatg

SEQ ID NO: 37 - DNA, 21 bp, Artificial Sequence
  Description of Artificial Sequence synthetic
gtcacagaaacatagcaagcc

SEQ ID NO: 38 - DNA, 21 bp, Artificial Sequence
  Description of Artificial Sequence synthetic
caaccaatataggagctaggc

SEQ ID NO: 39 - DNA, 22 bp, Artificial Sequence
  Description of Artificial Sequence synthetic
acatcctgagtactaaatgcag

SEQ ID NO: 40 - DNA, 23 bp, Artificial Sequence
  Description of Artificial Sequence synthetic
aaagcatgtgatatattcgtagg

SEQ ID NO: 41 - DNA, 24 bp, Artificial Sequence
  Description of Artificial Sequence synthetic
tttctctaatttgttttgttttgc

SEQ ID NO: 42 - DNA, 21 bp, Artificial Sequence
  Description of Artificial Sequence synthetic
tttagtttgactcaccttccc

SEQ ID NO: 43 - DNA, 21 bp, Artificial Sequence
  Description of Artificial Sequence synthetic
ccttccagatccataactcag

SEQ ID NO: 44 - DNA, 22 bp, Artificial Sequence
  Description of Artificial Sequence synthetic
taccttctagccaaagtaatcc

SEQ ID NO: 45 - DNA, 22 bp, Artificial Sequence
  Description of Artificial Sequence synthetic
aagtgtaaagtgaggaccaaac

SEQ ID NO: 46 - DNA, 21 bp, Artificial Sequence
  Description of Artificial Sequence synthetic
ttcatgaggcttcacagtagg

SEQ ID NO: 47 - DNA, 21 bp, Artificial Sequence
  Description of Artificial Sequence synthetic
ttcatgaggcttcacagtagg

SEQ ID NO: 48 - DNA, 23 bp, Artificial Sequence
  Description of Artificial Sequence synthetic
ttgaaaggaatctatgatctagg

SEQ ID NO: 49 - DNA, 21 bp, Artificial Sequence
  Description of Artificial Sequence synthetic
ttcttctcattgcagaacaca

SEQ ID NO: 50 - DNA, 20 bp, Artificial Sequence
  Description of Artificial Sequence synthetic
tgtgaaagtgtgctcaccac

SEQ ID NO: 51 - DNA, 22 bp, Artificial Sequence
  Description of Artificial Sequence synthetic
gatctgtgaactcttgttttca

SEQ ID NO: 52 - DNA, 21 bp, Artificial Sequence
  Description of Artificial Sequence synthetic
gaagagagacttacattaggc

SEQ ID NO: 53 - DNA, 22 bp, Artificial Sequence
  Description of Artificial Sequence synthetic
catgaaccattcttagcttctg

SEQ ID NO: 54 - DNA, 22 bp, Artificial Sequence
  Description of Artificial Sequence synthetic
ttgtgtcatctttactctcctg

SEQ ID NO: 55 - DNA, 23 bp, Artificial Sequence
  Description of Artificial Sequence synthetic
atgtgattatggaataggttgtc

SEQ ID NO: 56 - DNA, 23 bp, Artificial Sequence
  Description of Artificial Sequence synthetic
tatttaacatctcatacagtcag

SEQ ID NO: 57 - DNA, 21 bp, Artificial Sequence
  Description of Artificial Sequence synthetic
ccaaggactgttgaaagtagc

SEQ ID NO: 58 - DNA, 21 bp, Artificial Sequence
  Description of Artificial Sequence synthetic
ttgcatatgcaagtgtacagc

SEQ ID NO: 59 - DNA, 20 bp, Artificial Sequence
  Description of Artificial Sequence synthetic
cacagggttgttgttaagcc

SEQ ID NO: 60 - DNA, 21 bp, Artificial Sequence
  Description of Artificial Sequence synthetic
tctgaggatgtttccactttc

SEQ ID NO: 61 - DNA, 23 bp, Artificial Sequence
  Description of Artificial Sequence synthetic
ttatggctttgaagtatgagtta

SEQ ID NO: 62 - DNA, 21 bp, Artificial Sequence
  Description of Artificial Sequence synthetic
gcatgcttgacagtttctgag

SEQ ID NO: 63 - DNA, 21 bp, Artificial Sequence
  Description of Artificial Sequence synthetic
ctcagaaactgtcaagcatgc

SEQ ID NO: 64 - DNA, 19 bp, Artificial Sequence
  Description of Artificial Sequence synthetic
ttggaacggccaccaagac

SEQ ID NO: 65 - DNA, 19 bp, Artificial Sequence
  Description of Artificial Sequence synthetic
cccttctaaccatggccag

SEQ ID NO: 66 - DNA, 19 bp, Artificial Sequence
  Description of Artificial Sequence synthetic
gtgcctcctgtcaatggtg

SEQ ID NO: 67 - DNA, 19 bp, Artificial Sequence
  Description of Artificial Sequence synthetic
ctactgaaaccgcagcatg

SEQ ID NO: 68 - DNA, 19 bp, Artificial Sequence
  Description of Artificial Sequence synthetic
ttggagacagtgactcacc

SEQ ID NO: 69 - DNA, 21 bp, Artificial Sequence
  Description of Artificial Sequence synthetic
gctcattcgagtagcggctct

SEQ ID NO: 70 - DNA, 21 bp, Artificial Sequence
  Description of Artificial Sequence synthetic
ctcattcgagyagcggctctt

SEQ ID NO: 71 - DNA, 20 bp, Artificial Sequence
  Description of Artificial Sequence synthetic
agagccgctactcgaatgag

SEQ ID NO: 72 - DNA, 20 bp, Artificial Sequence
  Description of Artificial Sequence synthetic
agagccgctrctcgaatgag

SEQ ID NO: 73 - DNA, 21 bp, Artificial Sequence
  Description of Artificial Sequence synthetic
ctcattcgagcagcggctctt

SEQ ID NO: 74 - DNA, 20 bp, Artificial Sequence
  Description of Artificial Sequence synthetic
agagccgctgctcgaatgag

SEQ ID NO: 75 - DNA, 23 bp, Artificial Sequence
  Description of Artificial Sequence synthetic
cttcaggtcgggatggatcttga

SEQ ID NO: 76 - DNA, 23 bp, Artificial Sequence
  Description of Artificial Sequence synthetic
cttcaggtcggratggatcttga

SEQ ID NO: 77 - DNA, 20 bp, Artificial Sequence
  Description of Artificial Sequence synthetic
caagatccatcccgacctga

SEQ ID NO: 78 - DNA, 20 bp, Artificial Sequence
  Description of Artificial Sequence synthetic
caagatccatyccgacctga

SEQ ID NO: 79 - DNA, 23 bp, Artificial Sequence
  Description of Artificial Sequence synthetic
cttcaggtcggaatggatcttga

SEQ ID NO: 80 - DNA, 20 bp, Artificial Sequence
  Description of Artificial Sequence synthetic
caagatccattccgacctga

SEQ ID NO: 81 - DNA, 20 bp, Artificial Sequence
  Description of Artificial Sequence synthetic
aaactgaacaataaaaggta

SEQ ID NO: 82 - DNA, 20 bp, Artificial Sequence
  Description of Artificial Sequence synthetic
aaactgaacrataaaaggta

SEQ ID NO: 83 - DNA, 22 bp, Artificial Sequence
  Description of Artificial Sequence synthetic
taccttttattgttcagtttaa

SEQ ID NO: 84 - DNA, 22 bp, Artificial Sequence
  Description of Artificial Sequence synthetic
taccttttatygttcagtttaa

SEQ ID NO: 85 - DNA, 20 bp, Artificial Sequence
  Description of Artificial Sequence synthetic
aaactgaacgataaaagtgc

SEQ ID NO: 86 - DNA, 22 bp, Artificial Sequence
  Description of Artificial Sequence synthetic
taccttttatcgttcagtttaa

SEQ ID NO: 87 - DNA, 22 bp, Artificial Sequence
  Description of Artificial Sequence synthetic
gacataaatggtatgtttgttt

SEQ ID NO: 88 - DNA, 21 bp, Artificial Sequence
  Description of Artificial Sequence synthetic
agacataaatgktatgtttgt

SEQ ID NO: 89 - DNA, 22 bp, Artificial Sequence
  Description of Artificial Sequence synthetic
aacaaacataccatttatgtct

SEQ ID NO: 90 - DNA, 21 bp, Artificial Sequence
  Description of Artificial Sequence synthetic
aacaaacatamcatttatgtc

SEQ ID NO: 91 - DNA, 21 bp, Artificial Sequence
  Description of Artificial Sequence synthetic
agacataaatgttatgtttgt

SEQ ID NO: 92 - DNA, 21 bp, Artificial Sequence
  Description of Artificial Sequence synthetic
aacaaacataacatttatgtc

SEQ ID NO: 93 - DNA, 21 bp, Artificial Sequence
  Description of Artificial Sequence synthetic
gatacagggttcttcatgaat

SEQ ID NO: 94 - DNA, 21 bp, Artificial Sequence
  Description of Artificial Sequence synthetic
gatacagggtycttcatgaat

SEQ ID NO: 95 - DNA, 21 bp, Artificial Sequence
  Description of Artificial Sequence synthetic
attcatgaagaaccctgtatc

SEQ ID NO: 96 - DNA, 21 bp, Artificial Sequence
  Description of Artificial Sequence synthetic
attcatgaagraccctgtatc

SEQ ID NO: 97 - DNA, 21 bp, Artificial Sequence
  Description of Artificial Sequence synthetic
gatacagggtccttcatgaat

SEQ ID NO: 98 - DNA, 21 bp, Artificial Sequence
  Description of Artificial Sequence synthetic
attcatgaaggaccctgtatc

SEQ ID NO: 99 - DNA, 23 bp, Artificial Sequence
  Description of Artificial Sequence synthetic
taagcagcaacaatgtcgtgtgc

SEQ ID NO: 100 - DNA, 23 bp, Artificial Sequence
  Description of Artificial Sequence synthetic
taagcagcaayaatgtcgtgtgc

SEQ ID NO: 101 - DNA, 19 bp, Artificial Sequence
  Description of Artificial Sequence synthetic
agcagcaataatgtcgtgt

SEQ ID NO: 102 - DNA, 19 bp, Artificial Sequence
  Description of Artificial Sequence synthetic
ttcacttcagttacccatc

SEQ ID NO: 103 - DNA, 18 bp, Artificial Sequence
  Description of Artificial Sequence synthetic
tcacttcarttacccatc

SEQ ID NO: 104 - DNA, 18 bp, Artificial Sequence
  Description of Artificial Sequence synthetic
atgggtaactgaagtgaa

SEQ ID NO: 105 - DNA, 18 bp, Artificial Sequence
  Description of Artificial Sequence synthetic
atgggtaaytgaagtgaa

SEQ ID NO: 106 - DNA, 18 bp, Artificial Sequence
  Description of Artificial Sequence synthetic
tcacttcaattacccatc

SEQ ID NO: 107 - DNA, 18 bp, Artificial Sequence
  Description of Artificial Sequence synthetic
atgggtaattgaagtgaa

SEQ ID NO: 108 - DNA, 20 bp, Artificial Sequence
  Description of Artificial Sequence synthetic
cttgaagggcctgaacctga

SEQ ID NO: 109 - DNA, 20 bp, Artificial Sequence
  Description of Artificial Sequence synthetic
tcttgaagggyctgaacctg

SEQ ID NO: 110 - DNA, 20 bp, Artificial Sequence
  Description of Artificial Sequence synthetic
caggttcaggcccttcaaga

SEQ ID NO: 111 - DNA, 21 bp, Artificial Sequence
  Description of Artificial Sequence synthetic
tcaggttcagrcccttcaaga

SEQ ID NO: 112 - DNA, 18 bp, Artificial Sequence
  Description of Artificial Sequence synthetic
cttgaagggtctgaacct

SEQ ID NO: 113 - DNA, 17 bp, Artificial Sequence
  Description of Artificial Sequence synthetic
ggttcagacccttcaag

SEQ ID NO: 114 - DNA, 17 bp, Artificial Sequence
  Description of Artificial Sequence synthetic
tcagcagtcacattgca

SEQ ID NO: 115 - DNA, 17 bp, Artificial Sequence
  Description of Artificial Sequence synthetic
cagcagtyacattgcac

SEQ ID NO: 116 - DNA, 17 bp, Artificial Sequence
  Description of Artificial Sequence synthetic
cagcagttacattgcac

SEQ ID NO: 117 - DNA, 20 bp, Artificial Sequence
  Description of Artificial Sequence synthetic
caaaaattagtaaaggaata

SEQ ID NO: 118 - DNA, 20 bp, Artificial Sequence
  Description of Artificial Sequence synthetic
acaaaaattwgtaaaggaat

SEQ ID NO: 119 - DNA, 20 bp, Artificial Sequence
  Description of Artificial Sequence synthetic
caaaaatttgtaaaggaata

SEQ ID NO: 120 - DNA, 16 bp, Artificial Sequence
  Description of Artificial Sequence synthetic
gaagagatcgtgaggg

SEQ ID NO: 121 - DNA, 17 bp, Artificial Sequence
  Description of Artificial Sequence synthetic
gaagagatygtgagggc

SEQ ID NO: 122 - DNA, 17 bp, Artificial Sequence
  Description of Artificial Sequence synthetic
aagagattgtgagggca

SEQ ID NO: 123 - DNA, 19 bp, Artificial Sequence
  Description of Artificial Sequence synthetic
ggtttctcttcaggtcggg

SEQ ID NO: 124 - DNA, 20 bp, Artificial Sequence
  Description of Artificial Sequence synthetic
cggtttctcttcaggtcgga

SEQ ID NO: 125 - DNA, 20 bp, Artificial Sequence
  Description of Artificial Sequence synthetic
ctcagccaacaaacttctgc

SEQ ID NO: 126 - DNA, 24 bp, Artificial Sequence
  Description of Artificial Sequence synthetic
gaaacaagctagttaccttttatt

SEQ ID NO: 127 - DNA, 23 bp, Artificial Sequence
  Description of Artificial Sequence synthetic
aaacaagctagttaccttttatc

SEQ ID NO: 128 - DNA, 21 bp, Artificial Sequence
  Description of Artificial Sequence synthetic
tcttactgctctctgggcttc

SEQ ID NO: 129 - DNA, 22 bp, Artificial Sequence
  Description of Artificial Sequence synthetic
gaccaccacaaaacaaacatac

SEQ ID NO: 130 - DNA, 22 bp, Artificial Sequence
  Description of Artificial Sequence synthetic
gaccaccacaaaacaaacataa

SEQ ID NO: 131 - DNA, 20 bp, Artificial Sequence
  Description of Artificial Sequence synthetic
cacacagtcagcagagaagt

SEQ ID NO: 132 - DNA, 21 bp, Artificial Sequence
  Description of Artificial Sequence synthetic
cttcctccagattcatgaaga

SEQ ID NO: 133 - DNA, 21 bp, Artificial Sequence
  Description of Artificial Sequence synthetic
cttcctccagattcatgaagg

SEQ ID NO: 134 - DNA, 20 bp, Artificial Sequence
  Description of Artificial Sequence synthetic
cacacagtcagcagagaagt

SEQ ID NO: 135 - DNA, 21 bp, Artificial Sequence
  Description of Artificial Sequence synthetic
aaaaggatgcacacgacattg

SEQ ID NO: 136 - DNA, 22 bp, Artificial Sequence
  Description of Artificial Sequence synthetic
caaaaggatgcacacgacatta

SEQ ID NO: 137 - DNA, 20 bp, Artificial Sequence
  Description of Artificial Sequence synthetic
ggaatgagtggtctctttgg

SEQ ID NO: 138 - DNA, 23 bp, Artificial Sequence
  Description of Artificial Sequence synthetic
gaattcagaaatgttcacttcag

SEQ ID NO: 139 - DNA, 23 bp, Artificial Sequence
  Description of Artificial Sequence synthetic
gaattcagaaatgttcacttcaa

SEQ ID NO: 140 - DNA, 23 bp, Artificial Sequence
  Description of Artificial Sequence synthetic
actaggtttaaatatacatgcac

SEQ ID NO: 141 - DNA, 20 bp, Artificial Sequence
  Description of Artificial Sequence synthetic
cctggtagatcttgaagggc

SEQ ID NO: 142 - DNA, 20 bp, Artificial Sequence
  Description of Artificial Sequence synthetic
cctggtagatcttgaagggt

SEQ ID NO: 143 - DNA, 21 bp, Artificial Sequence
  Description of Artificial Sequence synthetic
gggcaacatcagaaagatgtg

SEQ ID NO: 144 - DNA, 23 bp, Artificial Sequence
  Description of Artificial Sequence synthetic
aggataggatatattcctttact

SEQ ID NO: 145 - DNA, 23 bp, Artificial Sequence
  Description of Artificial Sequence synthetic
aggataggatatattcctttaca

SEQ ID NO: 146 - DNA, 19 bp, Artificial Sequence
  Description of Artificial Sequence synthetic
tgggcatcacacttacccc

SEQ ID NO: 147 - DNA, 19 bp, Artificial Sequence
  Description of Artificial Sequence synthetic
ctcctttgctgccctcacg

SEQ ID NO: 148 - DNA, 19 bp, Artificial Sequence
  Description of Artificial Sequence synthetic
ctcctttgctgccctcaca

SEQ ID NO: 149 - DNA, 22 bp, Artificial Sequence
  Description of Artificial Sequence synthetic
gatctgtgaactcttgttttca

SEQ ID NO: 150 - DNA, 19 bp, Artificial Sequence
  Description of Artificial Sequence synthetic
gactaaagagacataaatg

SEQ ID NO: 151 - DNA, 19 bp, Artificial Sequence
  Description of Artificial Sequence synthetic
gactaaagasacataaatg

SEQ ID NO: 152 - DNA, 19 bp, Artificial Sequence
  Description of Artificial Sequence synthetic
catttatgtctctttagtc

SEQ ID NO: 153 - DNA, 19 bp, Artificial Sequence
  Description of Artificial Sequence synthetic
catttatgtstctttagtc

SEQ ID NO: 154 - DNA, 19 bp, Artificial Sequence
  Description of Artificial Sequence synthetic
gactaaagacacataaatg

SEQ ID NO: 155 - DNA, 19 bp, Artificial Sequence
  Description of Artificial Sequence synthetic
catttatgtgtctttagtc

SEQ ID NO: 156 - DNA, 19 bp, Artificial Sequence
  Description of Artificial Sequence synthetic
atcattaaatgaaatgagt

SEQ ID NO: 157 - DNA, 19 bp, Artificial Sequence
  Description of Artificial Sequence synthetic
atcattaaaygaaatgagt

SEQ ID NO: 158 - DNA, 19 bp, Artificial Sequence
  Description of Artificial Sequence synthetic
actcatttcatttaatgat

SEQ ID NO: 159 - DNA, 19 bp, Artificial Sequence
  Description of Artificial Sequence synthetic
actcatttcrtttaatgat

SEQ ID NO: 160 - DNA, 19 bp, Artificial Sequence
  Description of Artificial Sequence synthetic
atcattaaacgaaatgagt

SEQ ID NO: 161 - DNA, 19 bp, Artificial Sequence
  Description of Artificial Sequence synthetic
actcatttcgtttaatgat

SEQ ID NO: 162 - DNA, 19 bp, Artificial Sequence
  Description of Artificial Sequence synthetic
caacaatgtcgtgtgcatc

SEQ ID NO: 163 - DNA, 19 bp, Artificial Sequence
  Description of Artificial Sequence synthetic
caacaatgtygtgtgcatc

SEQ ID NO: 164 - DNA, 19 bp, Artificial Sequence
  Description of Artificial Sequence synthetic
gatgcacacgacattgttg

SEQ ID NO: 165 - DNA, 19 bp, Artificial Sequence
  Description of Artificial Sequence synthetic
gatgcacacracattgttg

SEQ ID NO: 166 - DNA, 19 bp, Artificial Sequence
  Description of Artificial Sequence synthetic
caacaatgttgtgtgcatc

SEQ ID NO: 167 - DNA, 19 bp, Artificial Sequence
  Description of Artificial Sequence synthetic
gatgcacacaacattgttg

SEQ ID NO: 168 - DNA, 19 bp, Artificial Sequence
  Description of Artificial Sequence synthetic
ggcttgaagatgtaagaat

SEQ ID NO: 169 - DNA, 19 bp, Artificial Sequence
  Description of Artificial Sequence synthetic
ggcttgaagrtgtaagaat

SEQ ID NO: 170 - DNA, 19 bp, Artificial Sequence
  Description of Artificial Sequence synthetic
attcttacatcttcaagcc

SEQ ID NO: 171 - DNA, 19 bp, Artificial Sequence
  Description of Artificial Sequence synthetic
attcttacaycttcaagcc

SEQ ID NO: 172 - DNA, 19 bp, Artificial Sequence
  Description of Artificial Sequence synthetic
ggcttgaaggtgtaagaat

SEQ ID NO: 173 - DNA, 19 bp, Artificial Sequence
  Description of Artificial Sequence synthetic
attcttacaccttcaagcc

SEQ ID NO: 174 - DNA, 19 bp, Artificial Sequence
  Description of Artificial Sequence synthetic
gaacattgcctatggagac

SEQ ID NO: 175 - DNA, 19 bp, Artificial Sequence
  Description of Artificial Sequence synthetic
gaacattgcytatggagac

SEQ ID NO: 176 - DNA, 19 bp, Artificial Sequence
  Description of Artificial Sequence synthetic
gtctccataggcaatgttc

SEQ ID NO: 177 - DNA, 19 bp, Artificial Sequence
  Description of Artificial Sequence synthetic
gtctccatargcaatgttc

SEQ ID NO: 178 - DNA, 19 bp, Artificial Sequence
  Description of Artificial Sequence synthetic
gaacattgcttatggagac

SEQ ID NO: 179 - DNA, 19 bp, Artificial Sequence
  Description of Artificial Sequence synthetic
gtctccataagcaatgttc

SEQ ID NO: 180 - DNA, 19 bp, Artificial Sequence
  Description of Artificial Sequence synthetic
aacttacttatatctttga

SEQ ID NO: 181 - DNA, 19 bp, Artificial Sequence
  Description of Artificial Sequence synthetic
aacttacttrtatctttga

SEQ ID NO: 182 - DNA, 19 bp, Artificial Sequence
  Description of Artificial Sequence synthetic
tcaaagatataagtaagtt

SEQ ID NO: 183 - DNA, 19 bp, Artificial Sequence
  Description of Artificial Sequence synthetic
tcaaagatayaagtaagtt

SEQ ID NO: 184 - DNA, 19 bp, Artificial Sequence
  Description of Artificial Sequence synthetic
aacttacttgtatctttga

SEQ ID NO: 185 - DNA, 19 bp, Artificial Sequence
  Description of Artificial Sequence synthetic
tcaaagatacaagtaagtt

SEQ ID NO: 186 - DNA, 19 bp, Artificial Sequence
  Description of Artificial Sequence synthetic
agaaatagtataatcaaca

SEQ ID NO: 187 - DNA, 19 bp, Artificial Sequence
  Description of Artificial Sequence synthetic
agaaatagtwtaatcaaca

SEQ ID NO: 188 - DNA, 19 bp, Artificial Sequence
  Description of Artificial Sequence synthetic
tgttgattatactatttct

SEQ ID NO: 189 - DNA, 19 bp, Artificial Sequence
  Description of Artificial Sequence synthetic
tgttgattawactatttct

SEQ ID NO: 190 - DNA, 19 bp, Artificial Sequence
  Description of Artificial Sequence synthetic
agaaatagtttaatcaaca

SEQ ID NO: 191 - DNA, 19 bp, Artificial Sequence
  Description of Artificial Sequence synthetic
tgttgattaaactatttct

SEQ ID NO: 192 - DNA, 19 bp, Artificial Sequence
  Description of Artificial Sequence synthetic
tagggagggtttaaggcca

SEQ ID NO: 193 - DNA, 19 bp, Artificial Sequence
  Description of Artificial Sequence synthetic
tagggagggyttaaggcca

SEQ ID NO: 194 - DNA, 19 bp, Artificial Sequence
  Description of Artificial Sequence synthetic
tggccttaaaccctcccta

SEQ ID NO: 195 - DNA, 19 bp, Artificial Sequence
  Description of Artificial Sequence synthetic
tggccttaarccctcccta

SEQ ID NO: 196 - DNA, 19 bp, Artificial Sequence
  Description of Artificial Sequence synthetic
tagggagggcttaaggcca

SEQ ID NO: 197 - DNA, 19 bp, Artificial Sequence
  Description of Artificial Sequence synthetic
tggccttaagccctcccta

SEQ ID NO: 198 - DNA, 19 bp, Artificial Sequence
  Description of Artificial Sequence synthetic
gaaaggtgagataaagcaa

SEQ ID NO: 199 - DNA, 19 bp, Artificial Sequence
  Description of Artificial Sequence synthetic
gaaaggtgarataaagcaa

SEQ ID NO: 200 - DNA, 19 bp, Artificial Sequence
  Description of Artificial Sequence synthetic
ttgctttatctcacctttc

SEQ ID NO: 201 - DNA, 19 bp, Artificial Sequence
  Description of Artificial Sequence synthetic
ttgctttatytcacctttc

SEQ ID NO: 202 - DNA, 19 bp, Artificial Sequence
  Description of Artificial Sequence synthetic
gaaaggtgaaataaagcaa

SEQ ID NO: 203 - DNA, 19 bp, Artificial Sequence
  Description of Artificial Sequence synthetic
ttgctttatttcacctttc

SEQ ID NO: 204 - DNA, 19 bp, Artificial Sequence
  Description of Artificial Sequence synthetic
catttaccccagatggacc

SEQ ID NO: 205 - DNA, 19 bp, Artificial Sequence
  Description of Artificial Sequence synthetic
catttacccyagatggacc

SEQ ID NO: 206 - DNA, 19 bp, Artificial Sequence
  Description of Artificial Sequence synthetic
ggtccatctggggtaaatg

SEQ ID NO: 207 - DNA, 19 bp, Artificial Sequence
  Description of Artificial Sequence synthetic
ggtccatctrgggtaaatg

SEQ ID NO: 208 - DNA, 19 bp, Artificial Sequence
  Description of Artificial Sequence synthetic
catttaccctagatggacc

SEQ ID NO: 209 - DNA, 19 bp, Artificial Sequence
  Description of Artificial Sequence synthetic
ggtccatctagggtaaatg

SEQ ID NO: 210 - DNA, 19 bp, Artificial Sequence
  Description of Artificial Sequence synthetic
gaggcgggcggatcacgag

SEQ ID NO: 211 - DNA, 19 bp, Artificial Sequence
  Description of Artificial Sequence synthetic
gaggcgggcrgatcacgag

SEQ ID NO: 212 - DNA, 19 bp, Artificial Sequence
  Description of Artificial Sequence synthetic
ctcgtgatccgcccgcctc

SEQ ID NO: 213 - DNA, 19 bp, Artificial Sequence
  Description of Artificial Sequence synthetic
ctcgtgatcygcccgcctc

SEQ ID NO: 214 - DNA, 19 bp, Artificial Sequence
  Description of Artificial Sequence synthetic
gaggcgggcagatcacgag

SEQ ID NO: 215 - DNA, 19 bp, Artificial Sequence
  Description of Artificial Sequence synthetic
ctcgtgatctgcccgcctc

SEQ ID NO: 216 - DNA, 19 bp, Artificial Sequence
  Description of Artificial Sequence synthetic
ggagaatggtgtgaacccg

SEQ ID NO: 217 - DNA, 19 bp, Artificial Sequence
  Description of Artificial Sequence synthetic
ggagaatggygtgaacccg

SEQ ID NO: 218 - DNA, 19 bp, Artificial Sequence
  Description of Artificial Sequence synthetic
cgggttcacaccattctcc

SEQ ID NO: 219 - DNA, 19 bp, Artificial Sequence
  Description of Artificial Sequence synthetic
cgggttcacrccattctcc

SEQ ID NO: 220 - DNA, 19 bp, Artificial Sequence
  Description of Artificial Sequence synthetic
ggagaatggcgtgaacccg

SEQ ID NO: 221 - DNA, 19 bp, Artificial Sequence
  Description of Artificial Sequence synthetic
cgggttcacgccattctcc

SEQ ID NO: 222 - DNA, 19 bp, Artificial Sequence
  Description of Artificial Sequence synthetic
gaggcgggcggatcacgag

SEQ ID NO: 223 - DNA, 19 bp, Artificial Sequence
  Description of Artificial Sequence synthetic
gaggcgggcrgatcacgag

SEQ ID NO: 224 - DNA, 19 bp, Artificial Sequence
  Description of Artificial Sequence synthetic
ctcgtgatccgcccgcctc

SEQ ID NO: 225 - DNA, 19 bp, Artificial Sequence
  Description of Artificial Sequence synthetic
ctcgtgatcygcccgcctc

SEQ ID NO: 226 - DNA, 19 bp, Artificial Sequence
  Description of Artificial Sequence synthetic
atatggaagaaaattacaa

SEQ ID NO: 227 - DNA, 19 bp, Artificial Sequence
  Description of Artificial Sequence synthetic
ttgtaattttcttccatat

SEQ ID NO: 228 - DNA, 19 bp, Artificial Sequence
  Description of Artificial Sequence synthetic
aacacgggcattgatctga

SEQ ID NO: 229 - DNA, 19 bp, Artificial Sequence
  Description of Artificial Sequence synthetic
aacacgggcrttgatctga

SEQ ID NO: 230 - DNA, 19 bp, Artificial Sequence
  Description of Artificial Sequence synthetic
tcagatcaatgcccgtgtt

SEQ ID NO: 231 - DNA, 19 bp, Artificial Sequence
  Description of Artificial Sequence synthetic
tcagatcaaygcccgtgtt

SEQ ID NO: 232 - DNA, 19 bp, Artificial Sequence
  Description of Artificial Sequence synthetic
aacacgggcgttgatctga

SEQ ID NO: 233 - DNA, 19 bp, Artificial Sequence
  Description of Artificial Sequence synthetic
tcagatcaacgcccgtgtt

SEQ ID NO: 234 - DNA, 19 bp, Artificial Sequence
  Description of Artificial Sequence synthetic
tgtattaaatgcgaatccc

SEQ ID NO: 235 - DNA, 19 bp, Artificial Sequence
  Description of Artificial Sequence synthetic
tgtattaaaygcgaatccc

SEQ ID NO: 236 - DNA, 19 bp, Artificial Sequence
  Description of Artificial Sequence synthetic
gggattcgcatttaataca

SEQ ID NO: 237 - DNA, 19 bp, Artificial Sequence
  Description of Artificial Sequence synthetic
gggattcgcrtttaataca

SEQ ID NO: 238 - DNA, 19 bp, Artificial Sequence
  Description of Artificial Sequence synthetic
tgtattaaacgcgaatccc

SEQ ID NO: 239 - DNA, 19 bp, Artificial Sequence
  Description of Artificial Sequence synthetic
gggattcgcgtttaataca

SEQ ID NO: 240 - DNA, 19 bp, Artificial Sequence
  Description of Artificial Sequence synthetic
ttgaaagacgtgtctacat

SEQ ID NO: 241 - DNA, 19 bp, Artificial Sequence
  Description of Artificial Sequence synthetic
ttgaaagacrtgtctacat

SEQ ID NO: 242 - DNA, 19 bp, Artificial Sequence
  Description of Artificial Sequence synthetic
atgtagacacgtctttcaa

SEQ ID NO: 243 - DNA, 19 bp, Artificial Sequence
  Description of Artificial Sequence synthetic
atgtagacaygtctttcaa

SEQ ID NO: 244 - DNA, 19 bp, Artificial Sequence
  Description of Artificial Sequence synthetic
ttgaaagacatgtctacat

SEQ ID NO: 245 - DNA, 19 bp, Artificial Sequence
  Description of Artificial Sequence synthetic
atgtagacatgtctttcaa

SEQ ID NO: 246 - DNA, 19 bp, Artificial Sequence
  Description of Artificial Sequence synthetic
cgtgtctacataagttgaa

SEQ ID NO: 247 - DNA, 19 bp, Artificial Sequence
  Description of Artificial Sequence synthetic
cgtgtctacwtaagttgaa

SEQ ID NO: 248 - DNA, 19 bp, Artificial Sequence
  Description of Artificial Sequence synthetic
ttcaacttatgtagacacg

SEQ ID NO: 249 - DNA, 19 bp, Artificial Sequence
  Description of Artificial Sequence synthetic
ttcaacttawgtagacacg

SEQ ID NO: 250 - DNA, 19 bp, Artificial Sequence
  Description of Artificial Sequence synthetic
cgtgtctacttaagttgaa

SEQ ID NO: 251 - DNA, 19 bp, Artificial Sequence
  Description of Artificial Sequence synthetic
ttcaacttaagtagacacg

SEQ ID NO: 252 - DNA, 19 bp, Artificial Sequence
  Description of Artificial Sequence synthetic
atgtccccaatgattcagc

SEQ ID NO: 253 - DNA, 19 bp, Artificial Sequence
  Description of Artificial Sequence synthetic
atgtccccartgattcagc

SEQ ID NO: 254 - DNA, 19 bp, Artificial Sequence
  Description of Artificial Sequence synthetic
gctgaatcattggggacat

SEQ ID NO: 255 - DNA, 19 bp, Artificial Sequence
  Description of Artificial Sequence synthetic
gctgaatcaytggggacat

SEQ ID NO: 256 - DNA, 19 bp, Artificial Sequence
  Description of Artificial Sequence synthetic
atgtccccagtgattcagc

SEQ ID NO: 257 - DNA, 19 bp, Artificial Sequence
  Description of Artificial Sequence synthetic
gctgaatcactggggacat

SEQ ID NO: 258 - DNA, 19 bp, Artificial Sequence
  Description of Artificial Sequence synthetic
ccgggccgggagcagtcat

SEQ ID NO: 259 - DNA, 19 bp, Artificial Sequence
  Description of Artificial Sequence synthetic
ccgggccggragcagtcat

SEQ ID NO: 260 - DNA, 19 bp, Artificial Sequence
  Description of Artificial Sequence synthetic
atgactgctcccggcccgg

SEQ ID NO: 261 - DNA, 19 bp, Artificial Sequence
  Description of Artificial Sequence synthetic
atgactgctyccggcccgg

SEQ ID NO: 262 - DNA, 19 bp, Artificial Sequence
  Description of Artificial Sequence synthetic
ccgggccggaagcagtcat

SEQ ID NO: 263 - DNA, 19 bp, Artificial Sequence
  Description of Artificial Sequence synthetic
atgactgcttccggcccgg

SEQ ID NO: 264 - DNA, 19 bp, Artificial Sequence
  Description of Artificial Sequence synthetic
aaaatacttcggaaatttg

SEQ ID NO: 265 - DNA, 19 bp, Artificial Sequence
  Description of Artificial Sequence synthetic
aaaatacttyggaaatttg

SEQ ID NO: 266 - DNA, 19 bp, Artificial Sequence
  Description of Artificial Sequence synthetic
caaatttccgaagtatttt

SEQ ID NO: 267 - DNA, 19 bp, Artificial Sequence
  Description of Artificial Sequence synthetic
caaatttccraagtatttt

SEQ ID NO: 268 - DNA, 19 bp, Artificial Sequence
  Description of Artificial Sequence synthetic
aaaatactttggaaatttg

SEQ ID NO: 269 - DNA, 19 bp, Artificial Sequence
  Description of Artificial Sequence synthetic
caaatttccaaagtatttt

SEQ ID NO: 270 - DNA, 19 bp, Artificial Sequence
  Description of Artificial Sequence synthetic
gatacagggttcttcatga

SEQ ID NO: 271 - DNA, 19 bp, Artificial Sequence
  Description of Artificial Sequence synthetic
gatacagggytcttcatga

SEQ ID NO: 272 - DNA, 19 bp, Artificial Sequence
  Description of Artificial Sequence synthetic
tcatgaagaaccctgtatc

SEQ ID NO: 273 - DNA, 19 bp, Artificial Sequence
  r=a or g
tcatgaagarccctgtatc

SEQ ID NO: 274 - DNA, 19 bp, Artificial Sequence
  Description of Artificial Sequence
gatacagggctcttcatga

SEQ ID NO: 275 - DNA, 19 bp, Artificial Sequence
  Description of Artificial Sequence
tcatgaagagccctgtatc

SEQ ID NO: 276 - DNA, 19 bp, Artificial Sequence
  Description of Artificial Sequence synthetic
gtgcacgatgttggggagc

SEQ ID NO: 277 - DNA, 19 bp, Artificial Sequence
  Description of Artificial Sequence synthetic
gtgcacgatrttggggagc

SEQ ID NO: 278 - DNA, 19 bp, Artificial Sequence
  Description of Artificial Sequence synthetic
gctccccaacatcgtgcac

SEQ ID NO: 279 - DNA, 19 bp, Artificial Sequence
  Description of Artificial Sequence synthetic
gctccccaayatcgtgcac

SEQ ID NO: 280 - DNA, 19 bp, Artificial Sequence
  Description of Artificial Sequence synthetic
gtgcacgatattggggagc

SEQ ID NO: 281 - DNA, 19 bp, Artificial Sequence
  Description of Artificial Sequence synthetic
gctccccaatatcgtgcac

SEQ ID NO: 282 - DNA, 19 bp, Artificial Sequence
  Description of Artificial Sequence synthetic
cattaaatgaaggactggg

SEQ ID NO: 283 - DNA, 19 bp, Artificial Sequence
  Description of Artificial Sequence synthetic
cattaaatgraggactggg

SEQ ID NO: 284 - DNA, 19 bp, Artificial Sequence
  Description of Artificial Sequence synthetic
cccagtccttcatttaatg

SEQ ID NO: 285 - DNA, 19 bp, Artificial Sequence
  Description of Artificial Sequence synthetic
cccagtcctycatttaatg

SEQ ID NO: 286 - DNA, 19 bp, Artificial Sequence
  Description of Artificial Sequence synthetic
cattaaatggaggactggg

SEQ ID NO: 287 - DNA, 19 bp, Artificial Sequence
  Description of Artificial Sequence synthetic
cccagtcctccatttaatg

SEQ ID NO: 288 - DNA, 19 bp, Artificial Sequence
  Description of Artificial Sequence synthetic
tcctctgagaatgtgcagt

SEQ ID NO: 289 - DNA, 19 bp, Artificial Sequence
  Description of Artificial Sequence synthetic
tcctctgagratgtgcagt

SEQ ID NO: 290 - DNA, 19 bp, Artificial Sequence
  Description of Artificial Sequence synthetic
actgcacattctcagagga

SEQ ID NO: 291 - DNA, 19 bp, Artificial Sequence
  Description of Artificial Sequence synthetic
actgcacatyctcagagga

SEQ ID NO: 292 - DNA, 19 bp, Artificial Sequence
  Description of Artificial Sequence synthetic
tcctctgaggatgtgcagt

SEQ ID NO: 293 - DNA, 19 bp, Artificial Sequence
  Description of Artificial Sequence synthetic
actgcacatcctcagagga

SEQ ID NO: 294 - DNA, 19 bp, Artificial Sequence
  Description of Artificial Sequence synthetic
aaaattgctgtcactatct

SEQ ID NO: 295 - DNA, 19 bp, Artificial Sequence
  Description of Artificial Sequence synthetic
aaaattgctrtcactatct

SEQ ID NO: 296 - DNA, 19 bp, Artificial Sequence
  Description of Artificial Sequence synthetic
agatagtgacagcaatttt

SEQ ID NO: 297 - DNA, 19 bp, Artificial Sequence
  Description of Artificial Sequence synthetic
agatagtgayagcaatttt

SEQ ID NO: 298 - DNA, 19 bp, Artificial Sequence
  Description of Artificial Sequence synthetic
aaaattgctatcactatct

SEQ ID NO: 299 - DNA, 19 bp, Artificial Sequence
  Description of Artificial Sequence synthetic
agatagtgatagcaatttt

SEQ ID NO: 300 - DNA, 19 bp, Artificial Sequence
  Description of Artificial Sequence synthetic
gagcacaacagtccagctg

SEQ ID NO: 301 - DNA, 19 bp, Artificial Sequence
  Description of Artificial Sequence synthetic
gagcacaacrgtccagctg

SEQ ID NO: 302 - DNA, 19 bp, Artificial Sequence
  Description of Artificial Sequence synthetic
cagctggactgttgtgctc

SEQ ID NO: 303 - DNA, 19 bp, Artificial Sequence
  Description of Artificial Sequence synthetic
cagctggacygttgtgctc

SEQ ID NO: 304 - DNA, 19 bp, Artificial Sequence
  Description of Artificial Sequence synthetic
gagcacaacggtccagctg

SEQ ID NO: 305 - DNA, 19 bp, Artificial Sequence
  Description of Artificial Sequence synthetic
cagctggaccgttgtgctc

SEQ ID NO: 306 - DNA, 19 bp, Artificial Sequence
  Description of Artificial Sequence synthetic
tgggcagacggtggccctg

SEQ ID NO: 307 - DNA, 19 bp, Artificial Sequence
  Description of Artificial Sequence synthetic
tgggcagacrgtggccctg

SEQ ID NO: 308 - DNA, 19 bp, Artificial Sequence
  Description of Artificial Sequence synthetic
cagggccaccgtctgccca

SEQ ID NO: 309 - DNA, 19 bp, Artificial Sequence
  Description of Artificial Sequence synthetic
cagggccacygtctgccca

SEQ ID NO: 310 - DNA, 19 bp, Artificial Sequence
  Description of Artificial Sequence synthetic
tgggcagacagtggccctg

SEQ ID NO: 311 - DNA, 19 bp, Artificial Sequence
  Description of Artificial Sequence synthetic
cagggccactgtctgccca

SEQ ID NO: 312 - DNA, 19 bp, Artificial Sequence
  Description of Artificial Sequence synthetic
ctcgtcctggtagatcttg

SEQ ID NO: 313 - DNA, 19 bp, Artificial Sequence
  Description of Artificial Sequence synthetic
ctcgtcctgrtagatcttg

SEQ ID NO: 314 - DNA, 19 bp, Artificial Sequence
  Description of Artificial Sequence synthetic
caagatctaccaggacgag

SEQ ID NO: 315 - DNA, 19 bp, Artificial Sequence
  Description of Artificial Sequence synthetic
caagatctaycaggacgag

SEQ ID NO: 316 - DNA, 19 bp, Artificial Sequence
  Description of Artificial Sequence synthetic
ctcgtcctgatagatcttg

SEQ ID NO: 317 - DNA, 19 bp, Artificial Sequence
  Description of Artificial Sequence synthetic
caagatctatcaggacgag

SEQ ID NO: 318 - DNA, 19 bp, Artificial Sequence
  Description of Artificial Sequence synthetic
gacccatgcgagctagacc

SEQ ID NO: 319 - DNA, 19 bp, Artificial Sequence
  Description of Artificial Sequence synthetic
gacccatgcragctagacc

SEQ ID NO: 320 - DNA, 19 bp, Artificial Sequence
  Description of Artificial Sequence synthetic
ggtctagctcgcatgggtc

SEQ ID NO: 321 - DNA, 19 bp, Artificial Sequence
  Description of Artificial Sequence synthetic
ggtctagctygcatgggtc

SEQ ID NO: 322 - DNA, 19 bp, Artificial Sequence
  Description of Artificial Sequence synthetic
gacccatgcaagctagacc

SEQ ID NO: 323 - DNA, 19 bp, Artificial Sequence
  Description of Artificial Sequence synthetic
ggtctagcttgcatgggtc

SEQ ID NO: 324 - DNA, 19 bp, Artificial Sequence
  Description of Artificial Sequence synthetic
actttgtctaatctcctgc

SEQ ID NO: 325 - DNA, 19 bp, Artificial Sequence
  Description of Artificial Sequence synthetic
actttgtctratctcctgc

SEQ ID NO: 326 - DNA, 19 bp, Artificial Sequence
  Description of Artificial Sequence synthetic
gcaggagattagacaaagt

SEQ ID NO: 327 - DNA, 19 bp, Artificial Sequence
  Description of Artificial Sequence synthetic
gcaggagatyagacaaagt

SEQ ID NO: 328 - DNA, 19 bp, Artificial Sequence
  Description of Artificial Sequence synthetic
actttgtctgatctcctgc

SEQ ID NO: 329 - DNA, 19 bp, Artificial Sequence
  Description of Artificial Sequence synthetic
gcaggagatcagacaaagt

SEQ ID NO: 330 - DNA, 19 bp, Artificial Sequence
  Description of Artificial Sequence synthetic
aatcattttctgtgccaca

SEQ ID NO: 331 - DNA, 19 bp, Artificial Sequence
  Description of Artificial Sequence synthetic
aatcattttmtgtgccaca

SEQ ID NO: 332 - DNA, 19 bp, Artificial Sequence
  Description of Artificial Sequence synthetic
tgtggcacagaaaatgatt

SEQ ID NO: 333 - DNA, 19 bp, Artificial Sequence
  Description of Artificial Sequence synthetic
tgtggcacakaaaatgatt

SEQ ID NO: 334 - DNA, 19 bp, Artificial Sequence
  Description of Artificial Sequence synthetic
aatcattttatgtgccaca

SEQ ID NO: 335 - DNA, 19 bp, Artificial Sequence
  Description of Artificial Sequence synthetic
tgtggcacataaaatgatt

SEQ ID NO: 336 - DNA, 19 bp, Artificial Sequence
  Description of Artificial Sequence synthetic
tctactggtgtttgtctta

SEQ ID NO: 337 - DNA, 19 bp, Artificial Sequence
  Description of Artificial Sequence synthetic
tctactggtrtttgtctta

SEQ ID NO: 338 - DNA, 19 bp, Artificial Sequence
  Description of Artificial Sequence synthetic
taagacaaacaccagtaga

SEQ ID NO: 339 - DNA, 19 bp, Artificial Sequence
  Description of Artificial Sequence synthetic
taagacaaayaccagtaga

SEQ ID NO: 340 - DNA, 19 bp, Artificial Sequence
  Description of Artificial Sequence synthetic
tctactggtatttgtctta

SEQ ID NO: 341 - DNA, 19 bp, Artificial Sequence
  Description of Artificial Sequence synthetic
taagacaaataccagtaga

SEQ ID NO: 342 - DNA, 19 bp, Artificial Sequence
  Description of Artificial Sequence synthetic
ttaattggccattttggac

SEQ ID NO: 343 - DNA, 19 bp, Artificial Sequence
  Description of Artificial Sequence synthetic
ttaattggcyattttggac

SEQ ID NO: 344 - DNA, 19 bp, Artificial Sequence
  Description of Artificial Sequence synthetic
gtccaaaatggccaattaa

SEQ ID NO: 345 - DNA, 19 bp, Artificial Sequence
  Description of Artificial Sequence synthetic
gtccaaaatrgccaattaa

SEQ ID NO: 346 - DNA, 19 bp, Artificial Sequence
  Description of Artificial Sequence synthetic
ttaattggctattttggac

SEQ ID NO: 347 - DNA, 19 bp, Artificial Sequence
  Description of Artificial Sequence synthetic
gtccaaaatagccaattaa

SEQ ID NO: 348 - DNA, 19 bp, Artificial Sequence
  Description of Artificial Sequence synthetic
aattttctccttacgggtg

SEQ ID NO: 349 - DNA, 19 bp, Artificial Sequence
  Description of Artificial Sequence synthetic
aattttctcmttacgggtg

SEQ ID NO: 350 - DNA, 19 bp, Artificial Sequence
  Description of Artificial Sequence synthetic
cacccgtaaggagaaaatt

SEQ ID NO: 351 - DNA, 19 bp, Artificial Sequence
  Description of Artificial Sequence synthetic
cacccgtaakgagaaaatt

SEQ ID NO: 352 - DNA, 19 bp, Artificial Sequence
  Description of Artificial Sequence synthetic
aattttctcattacgggtg

SEQ ID NO: 353 - DNA, 19 bp, Artificial Sequence
  Description of Artificial Sequence synthetic
cacccgtaatgagaaaatt

SEQ ID NO: 354 - DNA, 19 bp, Artificial Sequence
  Description of Artificial Sequence synthetic
ttctccttacgggtgttag

SEQ ID NO: 355 - DNA, 19 bp, Artificial Sequence
  Description of Artificial Sequence synthetic
ttctccttaygggtgttag

SEQ ID NO: 356 - DNA, 19 bp, Artificial Sequence
  Description of Artificial Sequence synthetic
ctaacacccgtaaggagaa

SEQ ID NO: 357 - DNA, 19 bp, Artificial Sequence
  Description of Artificial Sequence synthetic
ctaacacccktaaggagaa

SEQ ID NO: 358 - DNA, 19 bp, Artificial Sequence
  Description of Artificial Sequence synthetic
ttctccttatgggtgttag

SEQ ID NO: 359 - DNA, 19 bp, Artificial Sequence
  Description of Artificial Sequence synthetic
ctaacacccataaggagaa

SEQ ID NO: 360 - DNA, 19 bp, Artificial Sequence
  Description of Artificial Sequence synthetic
tgaatgttcagtggctccg

SEQ ID NO: 361 - DNA, 19 bp, Artificial Sequence
  Description of Artificial Sequence synthetic
tgaatgttcmgtggctccg

SEQ ID NO: 362 - DNA, 19 bp, Artificial Sequence
  Description of Artificial Sequence synthetic
cggagccactgaacattca

SEQ ID NO: 363 - DNA, 19 bp, Artificial Sequence
  Description of Artificial Sequence synthetic
cggagccackgaacattca

SEQ ID NO: 364 - DNA, 19 bp, Artificial Sequence
  Description of Artificial Sequence synthetic
tgaatgttccgtggctccg

SEQ ID NO: 365 - DNA, 19 bp, Artificial Sequence
  Description of Artificial Sequence synthetic
cggagccacggaacattca

SEQ ID NO: 366 - DNA, 19 bp, Artificial Sequence
  Description of Artificial Sequence synthetic
cgggtggtgtcacaggaag

SEQ ID NO: 367 - DNA, 19 bp, Artificial Sequence
  Description of Artificial Sequence synthetic
cgggtggtgwcacaggaag

SEQ ID NO: 368 - DNA, 19 bp, Artificial Sequence
  Description of Artificial Sequence synthetic
cttcctgtgacaccacccg

SEQ ID NO: 369 - DNA, 19 bp, Artificial Sequence
  Description of Artificial Sequence synthetic
cttcctgtgwcaccacccg

SEQ ID NO: 370 - DNA, 19 bp, Artificial Sequence
  Description of Artificial Sequence synthetic
cgggtggtgacacaggaag

SEQ ID NO: 371 - DNA, 19 bp, Artificial Sequence
  Description of Artificial Sequence synthetic
cttcctgtgtcaccacccg

SEQ ID NO: 372 - protein, 6 aa, Artificial Sequence
  Description of Artificial Sequence synthetic
KLNDKS

SEQ ID NO: 373 - protein, 7 aa, Artificial Sequence
  Description of Artificial Sequence synthetic
DTGSFMN

SEQ ID NO: 374 - protein, 5 aa, Artificial Sequence
  Description of Artificial Sequence synthetic
HFNYP

SEQ ID NO: 375 - protein, 6 aa, Artificial Sequence
  Description of Artificial Sequence synthetic
DTGLFM

SEQ ID NO: 376 - DNA, 20 bp, Artificial Sequence
  Description of Artificial Sequence synthetic
aaactgaacgataaaaggta